(19) United States
(12) Patent Application Publication
(10) Pub. No.: US 2012/0148546 A1
(43) Pub. Date: Jun. 14, 2012

(54) PERICYTE PROGENITOR CELLS AND METHODS OF GENERATING AND USING SAME

(75) Inventors: Ayelet Dar-Oaknin, Givat Ada (IL); Joseph Itskovitz-Eldor, Haifa (IL)

(73) Assignee: Technion Research & Development Foundation Ltd., Haifa (IL)

(21) Appl. No.: 13/390,734

(22) PCT Filed: Aug. 17, 2010
(86) PCT No.: PCT/IL10/00669

§ 371 Date: Feb. 16, 2012

Related U.S. Application Data
(60) Provisional application No. 61/272,103, filed on Aug. 17, 2009.

(51) Int. Cl. A61K 35/12; A61P 9/00; C12N 5/0775

(52) U.S. Cl. 424/93.7; 435/325; 435/377

(57) ABSTRACT

Provided are methods of isolating pericyte progenitor cells from pluripotent stem cells such as embryonic stem cells and induced pluripotent stem cells, by isolating CD105+, CD73+ and/or CD105+/CD73+ cells from embryoid bodies and optionally by enriching the cells with CD31− cells. Also provided are methods of isolating endothelial cells and co-derivation of pericyte and endothelial cells progenitor cells from embryoid bodies, and methods of differentiating same for various therapeutic applications. In addition, the invention provides an isolated pericyte progenitor cell having an expression marker signature of CD105+/CD73+CD31−/alpha SMA−/CD133−/Flk-1−.

### FIELD AND BACKGROUND OF THE INVENTION

The present invention, in some embodiments thereof, relates to isolated pericyte and endothelial progenitor cells from pluripotent stem cells, and methods of generating and using same.

In adult tissues, the majority of blood vessels are composed of three layers including a luminal inner monolayer of endothelial cells (tunica intima), an intermediate muscular layer (tunica media) of smooth muscle cells (SMC) and an outer abluminal layer of fibroblast-like, adventitial cells and connective tissue components (tunica adventitia). Microvessels, including capillaries, precapillary arterioles, postcapillary venues and collecting venules are composed of internal endothelial layer surrounded by outer coverage of pericytes (aka Rouget cells or mural cells), which are also called mesangial cells in the kidney and Ito cells in the liver. Both perivascular SMC and pericytes have been shown to function as critical regulators of vascular development, stabilization, maturation, and remodeling mediated by TGF-β, PDGF-B, SP1 or Ang1 [Armulik et al., 2005, Circulation Research, 97: 512-523; Bergers and Song, 2005, Neuro-Oncology, 2005, 7: 452-464)].

Although related in function and anatomical localization pericytes can be distinguished from SMC based on their characteristic morphology, and specific cell marker expression. Thus, while SMC form a separate layer of the tunica media in blood vessels, pericytes are physically embedded within the endothelial basement membrane to promote mutual communication with the underlying endothelium. In addition, SMC and the majority of pericytes in multiple human and murine tissue types express α-smooth muscle actin (α-SMA), which is involved in regulation of vessel contractility.

Osteogenic and chondrogenic differentiation was demonstrated for bovine retinal pericytes using mouse experimental model (Farrington-Rock et al., 2004, Circulation, 110:2226-2232). In addition, myogenic pericytes were identified in human skeletal muscle blood vessels, which colonized the muscle of mice with muscular dystrophy (Dellavalle et al., 2007, Nature Cell Biology, 9: 255-267).

Crisan M., et al. (Cell Stem Cell 3, 301-313, 2008) describe isolation and culturing of human perivascular cells (pericytes) from various adult and fetal tissues which were shown capable of differentiating into myogenic, osteogenic, adipogenic and chondrogenic cell in vitro. The cultured pericytes stably expressed NG2, CD146, α-SMA, PDGF-Rβ and alkaline phosphatase but not markers of endothelial cells (CD34, CD144, CD31, and vWF), hematopoietic cells (CD45) or myogenic cells (myogenin, m-cadherin, myf-5 and Pax 7).

In vitro, the pluripotent differentiation capability of human embryonic stem cells (hESC), which self-renew indefinitely and more recently the rapidly growing platform of human induced pluripotent stem cells (iPSC) provides a powerful model system to study vasculogenic development.

Several studies have described protocols for derivation of endothelial and SMC from endothelial progenitor cells (Levenberg S., Nat Protocols, 5: 1115-1118, 2010), whereas few reports identified pathways regulating the emergence of vasculogenic progenitor cells and derivatives (Goldman O., et al., 2009, Stem Cells 27: 1750-1759; Daylon J., Nature Biotechnology, advanced online publication, 17 Jan. 2010; Bai., et al., 2010, Journal of Cellular Biochemistry 109: 363-374;) using either spontaneous or induced differentiation models of human pluripotent stem cells (PSC).

Levenberg S., et al. (PNAS USA 2002, 99:4391-96) describe selection of endothelial cells from human embryoid bodies by cell sorting (FACS) using monoclonal antibodies raised against the endothelial-specific marker PECAM-1. The selected, PECAM-1+ embryoid body-derived (EBD) cells exhibited endothelial-specific characteristics such as von Willebrand factor, VEGFR-2 and VE-cadherin surface markers.

Bryan and D'Amore 2008 (Methods in Enzymology, 443: 315-331) describe isolation of pericytes from bovine retina, and generation of capillary-like structures in collagen and Matrigel from mesenchymal stem cells and primary endothelial cells, which adopt a pericyte/VSMC phenotype on the basis of expression of differentiation-associated proteins such as smooth muscle α-actin (α-SMA) and NG2 proteoglycan.

WO03/087296 and U.S. Pat. No. 7,354,763 disclose a method for the in-vitro identification, isolation and culture of human vasculogenic progenitor cells which differentiate into vascular smooth muscle cells.

Additional background art includes Cho S K., et al. 2001 (Blood, 98: 3635-3642); Lindskog H., et al. 2006 (Arteriosclerosis, Thrombosis, and Vascular Biology, 2006, 26:1457-1464); Brachvogel et al., 2005 (Development 132: 2657-2668); Corselli et al., 2010 (Arterioscler Thromb. Vasc. Biol. 2010, 30: 1104-9); Bagley R G., Cancer Res. 2005, 65: 9741-50; US Patent Application 2007/0264239 (Huard J and Peault B M); Barbery T., et al., 2005, PLoS Medicine, 2: 554-560; US Patent Application 2004/0009589, US Patent Application 20060198827; US Patent Application 20050266556;

### SUMMARY OF THE INVENTION

According to an aspect of some embodiments of the present invention there is provided a method of isolating a pericyte progenitor cell from embryoid bodies, comprising:

(a) isolating CD105+, CD73+ and/or CD105+/CD73+ cells from the embryoid bodies, to thereby obtain CD105+, CD73+ and/or CD105+/CD73+ cells, and;

(b) culturing the CD105+, CD73+ and/or CD105+/CD73+ cells,

thereby isolating the pericyte progenitor cell from the embryoid bodies.

According to an aspect of some embodiments of the present invention there is provided a method of isolating a pericyte progenitor cell from pluripotent stem cells, comprising:

(a) generating embryoid bodies from the pluripotent stem cells,

(b) isolating CD105+, CD73+ and/or CD105+/CD73+ cells from the embryoid bodies, to thereby obtain CD105+, CD73+ and/or CD105+/CD73+ cells, and;

(c) culturing the CD105+, CD73+ and/or CD105+/CD73+ cells,

thereby isolating the pericyte progenitor cell from the pluripotent stem cells.

According to an aspect of some embodiments of the present invention there is provided a method of isolating an endothelial progenitor cell from embryoid bodies, comprising:

(a) isolating CD105+, CD73+ and/or CD105+/CD73+ cells from the embryoid bodies, to thereby obtain CD105+, CD73+ and/or CD105+/CD73+ cells, and;

(b) isolating CD31+/UEA-1+/Ve-cadherin+ cells from the CD105+, CD73+ and/or CD105+/CD73+ cells,

thereby isolating the endothelial progenitor cell from the pluripotent stem cells.

According to an aspect of some embodiments of the present invention there is provided a method of isolating an endothelial progenitor cell from pluripotent stem cells, comprising:

(a) generating embryoid bodies from the pluripotent stem cells,

(b) isolating CD105+, CD73+ and/or CD105+/CD73+ cells from the embryoid bodies, to thereby obtain CD105+, CD73+ and/or CD105+/CD73+ cells, and;

(c) isolating CD31+/UEA-1+/Ve-cadherin+ cells from the CD105+, CD73+ and/or CD105+/CD73+ cells,

thereby isolating the endothelial progenitor cell from the pluripotent stem cells.

According to some embodiments of the invention, the method further comprising: culturing the CD105+, CD73+ and/or CD105+/CD73+ cells for about one or two passages prior to the isolating the CD31+/UEA-1+/Ve-cadherin+ cells from the CD105+, CD73+ and/or CD105+/CD73+ cells.

According to an aspect of some embodiments of the present invention there is provided a method of co-derivation of pericyte and endothelial progenitor cells, comprising:

(a) isolating CD105+, CD73+ and/or CD105+/CD73+ cells from embryoid bodies, to thereby obtain CD105+, CD73+ and/or CD105+/CD73+ cells;

(b) isolating a CD31+/UEA-1+/Ve-cadherin+ cells from the CD105+, CD73+ and/or CD105+/CD73+ cells, to thereby isolate the endothelial progenitor cells;

(c) isolating CD31− cells from the CD105+, CD73+ and/or CD105+/CD73+ cells, to thereby isolate the pericyte progenitor cells;

thereby co-derivation of pericyte and endothelial progenitor cells.

According to an aspect of some embodiments of the present invention there is provided a method of co-derivation of pericyte and endothelial progenitor cells, comprising:

(a) generating embryoid bodies from pluripotent stem cells,

(b) isolating CD105+, CD73+ and/or CD105+/CD73+ cells from the embryoid bodies, to thereby obtain CD105+, CD73+ and/or CD105+/CD73+ cells;

(c) isolating CD31+/UEA-1+/VE-cadherin+ cells from the CD105+, CD73+ and/or CD105+/CD73+ cells, to thereby isolate the endothelial progenitor cells;

(d) isolating CD31− cells from the CD105+, CD73+ and/or CD105+/CD73+ cells, to thereby isolate the pericyte progenitor cells;

thereby co-derivation of pericyte and endothelial progenitor cells.

According to some embodiments of the invention, the method further comprising: culturing the CD31+/UEA-1+/Ve-cadherin+ cells, to thereby expand the endothelial progenitor cells.

According to some embodiments of the invention, the method further comprising: culturing the CD31− cells, to thereby expand the pericyte progenitor cells.

According to an aspect of some embodiments of the present invention there is provided an isolated pericyte progenitor cell generated according to the method of some embodiments of the invention.

According to an aspect of some embodiments of the present invention there is provided an isolated pericyte progenitor cell having a CD105+/CD31−/αSMA−/CD133−/Flk-1−, a CD73+/CD31−/αSMA−/CD133−/Flk-1- or a CD105+/CD73+CD31−/αSMA−/CD133−/Flk-1− signature.

According to an aspect of some embodiments of the present invention there is provided an isolated population of cells which comprises at least 85% of the isolated pericyte progenitor cells of some embodiments of the invention.

According to an aspect of some embodiments of the present invention there is provided an isolated population of cells comprising at least 85% of pericyte progenitor cells, the pericyte progenitor cells having a CD105+/CD31−/αSMA−, a CD73+/CD31−/αSMA− or a CD105+/CD73+/CD31−/αSMA− signature.

According to an aspect of some embodiments of the present invention there is provided a cell culture comprising a culture medium and the isolated pericyte progenitor cell of some embodiments of the invention, or the isolated population of cells of some embodiments of the invention.

According to an aspect of some embodiments of the present invention there is provided a method of generating osteoblast cells, comprising culturing the isolated pericyte progenitor cell of some embodiments of the invention, or the isolated population of cells of some embodiments of the invention in a culture medium which comprises β-glycerol-phosphate, Dexamethasone and ascorbic acid, thereby generating the osteoblast cells.

According to an aspect of some embodiments of the present invention there is provided a method of generating adipocyte cells, comprising culturing the isolated pericyte progenitor cell of some embodiments of the invention, or the isolated population of cells of some embodiments of the invention in a culture medium which comprises IBMX (3-isobutyl-1-methylxanthine), Dexamethasone and insulin, thereby generating the adipocyte cells.

According to an aspect of some embodiments of the present invention there is provided a method of generating chondrocyte cells, comprising culturing the isolated pericyte progenitor cell of some embodiments of the invention, or the isolated population of cells of some embodiments of the invention in a culture medium which comprises dexamethasone, ascorbic acid and TGFβ3, thereby generating the chondrocyte cells.

According to an aspect of some embodiments of the present invention there is provided a method of generating myoblast cells, comprising culturing the isolated pericyte progenitor cell of some embodiments of the invention, or the isolated population of cells of some embodiments of the invention in a culture medium which comprises horse serum, thereby generating the myoblast cells.

According to an aspect of some embodiments of the present invention there is provided a method of generating smooth muscle cells in vivo, comprising implanting the isolated pericyte progenitor cell of some embodiments of the invention, or the isolated population of cells of some embodiments of the invention in a subject in need thereof, thereby generating the smooth muscle cells in vivo.

According to some embodiments of the invention, the isolated pericyte progenitor cells are mixed with an extracellular matrix.

According to an aspect of some embodiments of the present invention there is provided an isolated population of pericyte and endothelial progenitor cells generated according to the method of some embodiments of the invention.

According to an aspect of some embodiments of the present invention there is provided an isolated population of endothelial progenitor cells generated according to the method of some embodiments of the invention.

According to an aspect of some embodiments of the present invention there is provided a cell culture comprising a medium and the isolated population of cells or some embodiments of the invention.

According to an aspect of some embodiments of the present invention there is provided a pharmaceutical composition comprising the isolated pericyte progenitor cell of some embodiments of the invention, the isolated population of pericyte progenitor cells of some embodiments of the invention, the cell culture of some embodiments of the invention, the isolated population of pericyte and endothelial progenitor cells of some embodiments of the invention, and/or the isolated population of endothelial progenitor cells of some embodiments of the invention, and a therapeutically acceptable carrier.

According to an aspect of some embodiments of the present invention there is provided a method of treating a pathology requiring vascular tissue regeneration and/or repair, comprising administering to a subject having the pathology the isolated pericyte progenitor cell of some embodiments of the invention, the isolated population of pericyte progenitor cells of some embodiments of the invention, the cell culture of some embodiments of the invention, the isolated population of pericyte and endothelial progenitor cells of some embodiments of the invention, and/or the isolated population of endothelial progenitor cells of some embodiments of the invention, or the pharmaceutical composition of some embodiments of the invention, thereby treating the pathology.

According to some embodiments of the invention, the method further comprising passaging the CD105+, CD73+ and/or CD105+/CD73+ cells for at least 2 passages to thereby expand a population of pericyte progenitor cells.

According to some embodiments of the invention, the method further comprising enriching the cells for CD105+/CD31−, CD73+/CD31− and/or CD105+/CD73+/CD31− cells.

According to some embodiments of the invention, enriching is effected by depleting CD31+ cells from the CD105+, CD73+ and/or CD105+/CD73+ cells.

According to some embodiments of the invention, the pericyte progenitor cell having a CD105+/CD73+/CD31−/αSMA− signature.

According to some embodiments of the invention, the pericyte progenitor cell having a CD105+/CD73+/CD31−/αSMA−/CD133− signature.

According to some embodiments of the invention, the pericyte progenitor cell having a CD105+/CD73+/CD31−/αSMA−/Flk1− or CD105+/CD73+/CD31−/αSMA−/CD133−/Flk1− signature.

According to some embodiments of the invention, the pericyte progenitor cell having a CD105+/CD73+/CD31−/αSMA−/NG2+, CD105+/CD73+/CD31−/αSMA−/CD133−/NG2+ or CD105+/CD73+/CD31−/αSMA−/CD133−/Flk1−/NG2+ signature.

According to some embodiments of the invention, the pericyte progenitor cell is CD146+.

According to some embodiments of the invention, the pericyte progenitor cell is CD90+.

According to some embodiments of the invention, the pericyte progenitor cell is Tie-1+/Tie-2+.

According to some embodiments of the invention, the isolated pericyte progenitor cell is capable of differentiation into at least two cell lineages of the cell lineages selected from the group consisting of osteoblasts, chondrocytes, myobloasts and apipocytes.

According to some embodiments of the invention, isolating the CD105+, CD73+ and/or CD105+/CD73+ cells from the embryoid bodies is performed between about day 4 to about day 26 of differentiation of the embryoid bodies.

According to some embodiments of the invention, isolating the CD105+, CD73+ and/or CD105+/CD73+ cells from the embryoid bodies is performed by cell sorting or using magnetic beads.

According to some embodiments of the invention, the pericyte progenitor cell exhibits a CD105+/CD73+/CD31−/αSMA− signature at any passage in culture from passage 1 to senescence.

According to some embodiments of the invention, the pericyte progenitor cell exhibits a CD105+/CD73+/CD31−/αSMA−/CD133−/Flk−/NG2+/CD146+/CD90+/Tie-1+/Tie-2+ signature at any passage in culture from passage 1 to senescence.

According to some embodiments of the invention, the differentiation into at least two cell lineages is maintained at any passage in culture from passage 1 to senescence.

According to some embodiments of the invention, the pericyte being substantially free of contact inhibition when cultured in a two dimensional culture dish.

According to some embodiments of the invention, the pericyte adopts a hill and valley morphology when cultured in a two dimensional culture dish. According to some embodiments of the invention, the culturing comprises passaging the pericyte progenitor cell every 3-8 days.

According to some embodiments of the invention, the culturing comprises about 7-9 passages.

According to some embodiments of the invention, the osteoblast cells are characterized by mineral deposits and massive calcium content.

According to some embodiments of the invention, the adipocyte cells are characterized by accumulation of lipid-rich vacuoles which are positive for Oil red staining.

According to some embodiments of the invention, the chondrocyte cells are characterized by positive von-Kossa staining.

According to some embodiments of the invention, the enriching the cells is effected prior to the culturing the CD105+, CD73+ and/or CD105+/CD73+ cells.

According to some embodiments of the invention, the pluripotent stem cells are embryonic stem cells.

According to some embodiments of the invention, the pluripotent stem cells induced pluripotent stem cells (iPS).

According to some embodiments of the invention, the embryoid bodies are human embryoid bodies.

According to some embodiments of the invention, isolating the CD105+ and/or the CD105+/CD73+ cells is effected using an anti CD105 antibody.

According to some embodiments of the invention, isolating the CD73+ and/or CD105+/CD73+ cells is effected using a CD73 antibody.

According to some embodiments of the invention, isolating the CD105+, CD73+ and/or CD105+/CD73+ cells from the embryoid bodies is performed between about day 7 to about day 26 of differentiation of the embryoid bodies.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B depict RT-PCR analyses of vasculogenic markers on developing human PSC-derived EBs. FIG. 1A—CD105 expression; FIG. 1B—CD31 expression. Kinetic of vascular and perivascular gene expression was detected by RT-PCR from day 1 to day 26 of EBs differentiation in EBs which spontaneously differentiated from pluripotent stem cells: H9.2 (human ESC line) and induced pluripotent stem cells (C3).

FIGS. 2A-P are dot plots depicting flow cytometry analyses showing the expression of CD105 and CD31 cells in the developing EBs. EBs were spontaneously formed (differentiated) from pluripotent stem cells: H9.2 (human ESC line; FIGS. 2A, 2C, 2E, 2G, 2I, 2K, 2M and 2O) and induced pluripotent stem cells (C3; FIGS. 2B, 2D, 2F, 2H, 2J, 2L, 2N and 2P) and FACS analysis was performed at the indicated days of EBs differentiation: Day 1 (FIGS. 2A-B), day 4 (FIGS. 2C-D), day 7 (FIGS. 2E-F), day 10 (FIGS. 2G-H), day 14 (FIGS. 2I-J), day 19 (FIG. 2K-L), day 21 (FIGS. 2M-N) and day 26 (FIG. 20-P). The Y axis in each panel represents CD105 expression in the dissociated EBs single cells, and the X axis in each panel represents CD31 expression in the dissociated EBs single cells.

FIGS. 3A-D are histograms depicting percentages of the subpopulations of cells in cells of differentiated EBs. Results are representative to 4 independent experiments using H9.2 and 3 independent experiments using C3 pluripotent stem cells. FIG. 3A—C3 EBs; FIG. 3B—C3 EBs; FIG. 3C—H9.2 EBs; FIG. 3D—H9.2 EBs. FIGS. 3A and 3C: White bars: CD31+/CD73−; black bars: CD31+/CD73+; and shaded (grey) bars: CD31−/CD73+. FIGS. 3B and 3D: White bars: CD105+/CD31+; black bars: CD105+/CD31−; and shaded (grey) bars: CD105−/CD31+. The results show the time course of emergence of CD31+CD105+/− endothelial population and non-endothelial CD105+CD31− subset during the onset of vasculogenesis in developing EBs.

FIGS. 4A-C are dot plots depicting flow cytometry analyses showing the emergence of pericytes in differentiated PSC-derived EBs. Co-expression of CD105 and CD90 on a CD31 negative subpopulation of 19 days old I6 hESC-derived EBs. Subsets of CD105+CD90−CD31− and CD105+CD90+CD31− can be identified by flow cytometry analysis.

FIGS. 5A-H are fluorescent microscopy images depicting immunofluorescence analyses of differentiating EBs using antibodies specific to the CD31 and CD90 cell surface markers. FIGS. 5A-B—CD31; FIGS. 5C-D—CD90; FIGS. 5E-F—double-staining of CD31 and CD90; FIGS. 5G-H—triple staining of CD31, CD90 and DAPI (which stains nuclei). Immunofluorescence labeling of KTR13 iPSC derived 17 days old EBs demonstrating CD31+ endothelial network formation coinciding with the emergence of CD90 positive cell clusters Magnification: ×50 for FIGS. 5A, 5B, 5C and 5D; ×400 for FIGS. 5E, 5F, 5G and 5H.

FIGS. 6A-H are fluorescent microscopy images depicting immunofluorescence analyses of differentiating EBs using antibodies specific to the Calopnin and CD90 cell surface markers. FIGS. 6A-B—Calopnin; FIGS. 6C-D—CD90; FIGS. 6E-F—double-staining of Calopnin and CD90; FIGS. 6G-H—triple staining of Calopnin, CD90 and DAPI (which stains nuclei). Immunofluorescence labeling of KTR13 iPSC derived 17 days old EBs demonstrating co-expression of CD90 with calponin, which are expressed by pericytes and mesenchymal stem cells. Magnification: ×50 for FIGS. 6A, 6B, 6C and 6D; ×400 for FIGS. 6E, 6F, 6G and 6H.

FIGS. 7A-I are microscopy images depicting characterization of PSC-derived colonies generated from isolated CD105+ single cells at day 7 (FIGS. 7A, 7D and 7G) and day 20 (FIGS. 7B, 7C, 7E, 7F, 7H and 7I). CD105+ cells were sorted from 14 days old EBs and sparsely-plated (3 cells/cm²) in M-199 ECs growth medium. Shown are phase microscopy images (FIGS. 7A, 7B, 7D, 7E, 7G and 7H) and fluorescence microscopy images (FIGS. 7C, 7F and 7I). FIG. 7A—EC colony day 7; FIG. 7B—EC colony day 20; FIG. 7C—Dill-ac-LDL uptake (red) day 20; FIG. 7D—EC-SMA colony day 7; FIG. 7E—EC-SMA colony day 20; FIG. 7F—CD31 (red), SMA (green) and DAPI (blue) staining colony day 20; FIG. 7G—Pericyte colony day 7; FIG. 7H—Pericyte colony day 20; FIG. 7I—SMA (red) and DAPI (blue) staining colony day 20. Three types of colonies are detected within 7 days in culture: (i) CD31⁺ EC colonies (FIGS. 7A-C) (ii) Mixed CD31+ endothelial and CD31-αSMA+ cells with rare subset expressing both markers (FIGS. 7D-F) and (iii) Non-ECs, of which the majority of cells were CD31⁻αSMA⁻ multilayered cells with a minor subset of α-SMA positive smooth muscle cells (FIGS. 7G-I).

FIG. 8 is a graph depicting population doublings (PD) during 60 days of continuous PSC-derived pericyte culture from both hESC H9.2 and human iPS C3, with a PDT=108 hours between weeks 1-2 and a faster growth rate between weeks 3-5, at which the PDT was about 60 hours.

FIG. 9 is a histogram depicting fold increase (expansion) in PSC-derived pericyte number (from both hESC H9.2 and human iPS C3) throughout 60 days of culture expansion (up to 8 passages).

FIGS. 10A-P are dot plots depicting flow cytometry analyses of cultured PSC-derived pericytes between passage 1-8, until senescence. The PSC-derived pericytes were isolated from EBs generated by spontaneous differentiation of hESC9.2 (FIGS. 10A, 10C, 10E, 10G, 10I, 10K, 10M and 10O) or hiPSC C3 cells (FIGS. 10B, 10D, 10F, 10H, 10J, 10L, 10N and 10P). The cells subjected to analysis were all CD31− (negative for expression of CD31), taken from any passage from the range of passages 1-8.

FIG. 11 is a gel image depicting RT-PCR analysis of cultured PSC-derived pericytes between passage 1-8, until senescence. The PSC-derived pericytes were isolated from EBs generated by spontaneous differentiation of hESC9.2 or hiPSC C3 cells. Lanes from left to right: (1) DNA marker; (2) Ctl (control); (3) αSMA; (4) Fibroblast specific protein-1 (fsp1); (5) CD73; (6) CD90; (7) CD105; (8) Calponin; Note expression of CD73, CD90, CD105 and Calponin. Consistently, throughout the whole culture, the majority of pericytes were negative for α-SMA (arrow) and only a small subset of pericytes (about 6%) was positive for α-SMA.

FIGS. 12A-F are fluorescence microscopy images depicting immunofluorescence analysis of cultured PSC-derived pericytes between passage 1-8, until senescence. The PSC-derived pericytes were isolated from EBs generated by spontaneous differentiation of hESC9.2 (FIGS. 12A, 12C, 12E) or hiPSC C3 cells (FIGS. 12B, 12D and 12F). FIGS. 12A-B—double staining of calponin (red) and PDGFR-β (green). DAPI stained nuclei in blue. FIGS. 12C-D—alpha-SMA (red) and NG2 (green). DAPI stained nuclei in blue. FIGS. 12E-F—CD90 (red) and CXCR4 (green). DAPI (4′,6-diamidino-2-phenylindole) stained nuclei in blue.

FIG. 13 is a dot plot depicting flow cytometry of the isolated pericyte cells in culture (from passage 1-9 or senescence) obtained according to the method of some embodiments of the invention using alpha smooth muscle actin (αSMA) antibodies (clone 1A4, DAKO). Note that while the majority of the cells are αSMA− (i.e., do not express αSMA), a small subset of the cells (up to 6%) express αSMA. Thus, the isolated population of cells includes at least 94% of αSMA− cells.

FIGS. 14A-B are fluorescence microscopy images depicting immunofluorescence using anti-Tie-1 (FIG. 14A, red) and anti-Tie-2 (FIG. 14B, green) antibodies. Nuclei were stained using DAPI. Tie-1+ (positive for Tie-1) cells are stained in red (with purple nuclei due to DAPI stain. Tie-2+ (positive for Tie-2) cells are stained in green (with blue nuclei due to DAPI stain). Note that all pericyte progenitor cells are Tie-1+/Tie-2+.

FIGS. 15A-B are light microscopy images depicting cord networks formation on Matrigel by PSC-derived pericytes at passage 6 (FIG. 15A) or EPCs (FIG. 15B). Pericytes rearrange in smaller tube-like structures in comparison to those created by EPCs. These results demonstrate that the pericyte cells generated according to some embodiments of the invention are capable of forming vasculogenic structures in vitro.

FIGS. 16A-H are microscopic images depicting immunofluorescence analysis of vasculogenic cells assembling into tubular network. Seeding a mixture of PSC-derived endothelial cells identified by immuno-labeling of CD31 (red, FIGS. 16A and 16E) and carboxyfluorescein diacetate succinimidyl ester (CFSE)-labeled PSC-derived pericytes (green, FIGS. 16B and 16F) on Matrigel result in efficient assembly of the vasculogenic cells into tubular network (merged, FIGS. 16D and 16H). Nuclear staining by DAPI (Blue, FIGS. 16c and 16G). Original magnifications: ×40, FIGS. 16A, 16B, 16C and 16D ×100, FIGS. 16E, 16F, 16G and 16H.

FIGS. 17A-E are light microscopy images depicting hematoxylin and eosine (H&E) stains stained sections of implants generated by the pericytes cells and/or endothelial cells of some embodiments of the invention. A total of 1.5×10⁶ pericytes (8×10⁵-10⁶) and EPC (5-7×10⁵) were re-suspended in 250 μl Matrigel without adding vasculogenic growth factors or serum to the mixture. Matrigel mixtures were then implanted subcutaneously into immunodeficient 6-8 weeks old NOD/SCID mice, harvested after 7 days, sectioned and stained with H&E. FIG. 17A—empty implant (control Matrigel implant); FIG. 17B—Matrigel mixed with PSC-derived EPC only (H9.2) (H9.2-derived EC); FIG. 17C—Matrigel mixed with PSC-derived pericytes only (H9.2-derived pericytes); FIG. 17D—Matrigel mixed with PSC-derived pericytes mixed with human umbilical vein endothelial cells (H9.2-derived pericytes+HUVECs); FIG. 17E—Matrigel mixed with PSC-derived pericytes and endothelial cells (both derived from C3) (iPSC C3-derived ECs and pericytes). H&E staining revealed the presence of luminal structures containing erythrocytes (red, un-nucleated cells) in implants where both vasculogenic cells types (endothelial and pericytes) were used (FIGS. 17B and 17C, but not in implants where endothelial cells or pericytes were used alone. Implants of pericytes only induced infiltration of very few murine blood vessel into the Matrigel (FIG. 17C).

FIGS. 18A-E are fluorescence microscopy images depicting immunofluorescence analysis of implants generated the pericytes cells and/or endothelial cells of some embodiments of the invention. A total of 1.5×10⁶ pericytes (8×10⁵-10⁶) and EPC (5-7×10⁵) were re-suspended in 250 μl Matrigel without adding vasculogenic growth factors or serum to the mixture. Matrigel mixtures were then implanted subcutaneously into immunodeficient 6-8 weeks old NOD/SCID mice, harvested after 7 days, sectioned and immunolabeled with antibodies specific for human over mouse vasculogenic markers. FIG. 18A—Matrigel mixed with iPSC-derived pericytes. MHC class I (red), nuclei (DAPI, blue); FIG. 18B—Matrigel mixed with PSC-derived pericytes mixed with human umbilical vein endothelial cells (H9.2-derived pericytes+HUVECs). CD31 (red), MHC class I (green), nuclei (DAPI, blue); FIG. 18C—Matrigel mixed with PSC-derived pericytes and endothelial cells (C3) (iPSC-derived ECs and pericytes). CD34 (red), MHC class I (green), nuclei (DAPI, blue); FIG. 18D—Matrigel mixed with H9.2-derived endothelial cells and pericytes. CD31 (red), vW factor (green), Nuclei (DAPI, blue); FIG. 18E—Matrigel mixed with PSC-derived pericytes and endothelial cells (C3) (iPSC-derived ECs and pericytes) CD31 (red), MHC class I (green), Nuclei (DAPI, blue); Cultured pericytes, expressing the human MHC Class I antigen (red, FIG. 18A) mediated formation of vasculogenic structures in vivo within 1 week. Luminal implanted endothelial cells (HUVECs, H9.2 or C3 derived endothelial cells) are immunoreactive for the specific antibody against human CD31 or CD34 endothelial cell marker.

FIGS. 19A-F are microscopy images depicting osteogenic characteristics of cultured PSC-derived pericytes in vitro and in vivo. FIGS. 19A-B—Cultured PSC-derived pericytes were stimulated with osteogenic medium for 2-4 weeks. Mineral deposits appear in black (FIG. 19A). Robust Alizarin red stained calcium deposits can be seen within 2 weeks in osteogenic medium (FIG. 19B). FIG. 19B—inset: monolayer of alizarin red stained differentiated PSC-derived pericytes in culture dish. Uniform osteogenic potential was identical throughout the whole culture period from passage 2 to passage 8 until cell senescence, indicating that osteogenic potential of the expanded pericytes was fully maintained. Original magnifications: FIG. 19A, ×100; FIG. 19B, ×160. FIGS. 19C-F—Cultured pericytes were cultivated in osteogenic medium for 3 (FIGS. 19C-D) or 14 days (FIGS. 19E-F), removed, mixed with Matrigel, and implanted subcutaneously in the back of SCID-NOD mice. H&E (FIG. 19C and FIG. 19E) demonstrate ectopic bone-like formation structures with infiltration of murine blood vessels. FIGS. 19D and 19F—Alizarin red staining reveals moderate (FIG. 19D, 3 days in osteogenic medium) to massive (FIG. 19F, 14 days in osteogenic medium) appearance of calcium deposits corresponding to of length incubation periods with osteogenic medium of implanted pericytes. Original magnifications: FIG. 19F, ×40; FIG. 19C, FIG. 19D and FIG. 19E ×200.

FIGS. 20A-B are microscopy images depicting adipogenic potential of PSC-derived pericytes in vitro. Cultured PSC-derived pericytes (passages 2-8) were stimulated with adipogenic medium up to 14 days fixed and stained with Oil red 0 for detection of lipids. H9.2-derived (FIG. 20A, ×100) and C3 derived (FIG. 20B, ×200) differentiated pericytes.

FIGS. 21A-J are flow cytometry analyses depicting VEGFR1/Flt-1, VEGFR2/Flk-1, CD31 and UEA-1 expression pattern of cultured endothelial cells (ECs; FIGS. 21B, 21D, 21F, 21H, 21J) and progenitor pericytes (FIGS. 21A, 21C, 21E, 21G, 21I) from pluripotent stem cells (PSCs). FIGS. 21A-B—negative controls using PE-conjugated matched isotype control (IgG) antibodies; FIGS. 21C-D—VEGFR1/Flt-1; FIGS. 21E-F—VEGFR2/Flk-1; FIGS. 21G-H—CD31; FIGS. 21I-J—UEA-1-FITC. The analyses are representative for passages 1-5 for ECs and 1-8 for pericytes. Note that while the pericyte progenitors are negative for Flt-1 (FIG. 21C), Flk-1 (FIG. 21E), CD31 (FIG. 21G) and Ulex europaeus agglutinin-1 (UEA-1) (FIG. 21I), the endothelial cells are positive for Flt-1 (FIG. 21D), Flk-1 (FIG. 21H), CD31 (FIG. 21H) and UEA-1 (FIG. 21J).

FIGS. 22A-C depict the potential usage of PSC-derived vasculogenic endothelial cells and pericytes in the clinic. FIG. 22A—photograph of a critical limb ischemia due to diabetes. FIGS. 22B-C—schematic illustrations of arteriosclerosis. Arteries become narrowed and blood flow decreases. Diabetes as well vascular diseases result in vascular cell loss and impaired blood-flow to the progressively damaged tissue (FIG. 22A-B). In order to successfully develop therapeutic vascularization and tissue engineering strategies for blood vessel repair and generation sufficient numbers of vasculogenic cells are needed, which can be expanded ex vivo. Robust numbers of pericytes and endothelial cells are needed for blood vessel regeneration. Endothelial cells and pericytes are mainly required for regeneration of microvessels and capillaries.

FIGS. 23A-E are schematic diagrams illustrating co-derivation of vasculogenic endothelial and pericytes from human PSC-derived embryoid bodies. Embryoid bodies are spontaneously formed in differentiating medium up to 26 days, 3D differentiating embryoid bodies in feeder free culture (FIG. 23A). Step 1: Enzymatic dissociation of EBs with Collagenase B and DNase I results in single cells, of which CD105+ are isolated or sorted for further culture (endothelial and pericyte progenitor cells). Co-cultivation of CD105+ endothelial and pericyte progenitor cells at passage 0 results in 2 cell types: vasculogenic pericytes and endothelial cells (FIG. 23B). Further culturing results in differentiation of pericyte progenitor cells into vasculogenic pericytes (FIG. 23C). At passage 1, a second isolation step (step 2) based on CD31+ gives rise to two distinguished population: CD31+UEA-1+CD105+ endothelial cells (FIG. 23D, positive fraction) and CD31-UEA-1-CD105+ pericytes (FIG. 23E, negative fraction).

FIGS. 24A-C are dot plots depicting flow cytometry analyses of CD105+ population in human ESC-derived EBs. Dissociated EBs (7 days old) were labeled with either PE-conjugated anti-CD105 (FIG. 24A, 3.6% CD31+ of total dissociated EBs), FITC-conjugated anti-CD31 (FIG. 24B, 3.2% CD31+ of total dissociated EBs) or combination of both (FIG. 24C). Subpopulations of CD105+CD31+ (3.1%), CD105+CD31− (0.4%) and CD105−/lowCD31+ (0.1%) can be seen.

FIGS. 25A-C are dot plots depicting flow cytometry analyses of CD31+ endothelial progenitor cells, demonstrating enhancement of the percentage of CD31+ endothelial cells after EB-dissociation with Collagenase B and DNase I. Representative percentages of CD31+ populations from 14 days old dissociated H9.2-derived EBs (FIGS. 25A and 25B). Cells were isolated by magnetic separation using anti-CD31 conjugated beads to yield at least 95% pure CD31+ cell population (FIG. 25C).

FIGS. 26A-E depict derivation of two distinguished vasculogenic populations from isolated cultured CD105+ cells at first passage. FIG. 26A—dot plot of flow cytometry analysis of CD105⁺ isolated cells using CD31 and VEGFR-PE antibodies; FIG. 26B—Immunofluorescence analysis of CD105+/CD31− or CD105+/UEA-1− using antibodies which stain NG2 (green) and Calponin (red), nuclei are stained with DAPI (blue); FIG. 26C-Immunofluorescence analysis of CD105+/CD31+ or CD105+/UEA-1+ cells using antibodies which stain vW factor (green) and CD31 (red), nuclei are stained with DAPI (blue); FIG. 26D—dot plot depicting flow cytometry analysis using CD31 and CD105 antibodies; FIG. 26E—dot plot depicting flow cytometry analysis using UEA-1 and VE-cadherin antibodies. The composition of CD105⁺ isolated cells was identified between passage 0 to passage 1 using antibodies against endothelial markers: CD31 and VEGFR1. CD31+/VEGFR1+ PSC-derived endothelial cells comprised the majority of cell population as opposed to the non-endothelial cells (ECs) CD31−/VEGFR1— population (FIG. 26A). Removal of the endothelial fraction by sorting or magnetic separation using anti-CD31 or by sorting using UEA-1-FITC-conjugated lectins between passage 0 to passage 1 and further culturing of the non-ECs population resulted in homogenous cell cultures of (i) pericytes, which are NG2+/Calponin+ (FIG. 26B), CD105+/CD31− (FIG. 26D) and VE-Cadherin−/UEA-1− (FIG. 26E) PSC-derived pericytes (FIG. 26E); and (ii) vW Factor+/CD31+ (FIG. 26C), CD31+/CD105+ (FIG. 26D) and UEA-1+/VE-Cadherin+/low (FIG. 26E) PSC-derived endothelial cells.

FIGS. 27A-E are microscopy images (FIGS. 27A-D) and a dot plot (FIG. 27E) demonstrating that cultured CD105+/CD31+ PSC-derived cells exhibit stable endothelial characteristics in vitro. Isolated CD105+/CD31+, cultured in M-199 supplemented with 20% FBS, 50 μg/ml ECGS and 20 U/ml heparin present features of vasculogenic endothelial in vitro including: tube formation on Matrigel (FIG. 27A), stable expression of specific endothelial cell markers UEA-1 (FIG. 27B) or CD31 (FIG. 27C) and could be expanded up to passage 5 (FIG. 27D). For example 2×10⁶ CD105⁺ endothelial cells were isolated from 1×10⁸ cells of dissociated iPSC-derived EBs and expanded as follows: 3.5×10⁶ after second isolation step from pericytes-endothelial cells mixed culture, 7×10⁶ at passage 2, 1.5×10⁷ at passage 3, 5×10 at passage 4, and 1×10⁸ at passage 5. Flow cytometry analysis shows that cultivated CD105⁺ endothelial cells maintained CD31 expression (FIG. 27E).

FIG. 28 is a dot plot depicting flow cytometry analysis of CD31 of EBs cells generated from human foreskin-derived iPSCs demonstrating derivation of endothelial cells from iPSC. Human foreskin fibroblasts were used for generation of iPSC. Dissociated EBs from iPSC (14 days old) contain CD31⁺ endothelial population.

FIGS. 29A-F are microscopy images depicting immunofluorescence of CD105+ cells-derived pericytes. Cultivated CD105+/CD31− PSC-derived pericytes exhibit pericytic markers in vitro. Immuno-labeling of cultured pericytes revealed co-expression of Calponin (red, FIGS. 29A and 29D) and NG2 (green, FIGS. 29B and 29E). FIGS. 29C and 29F display merged images of calponin, NG2 with nuclear staining with DAPI (blue). Original magnifications: ×200 in FIGS. 29A-C; and ×630 in FIGS. 29D-F.

FIGS. 30A-C are dot plots (FIG. 30A-B) and a microscopy image (FIG. 30C) depicting representative phenotypes of cultured pericytes from passage 1 to passage 9 or during senescence. FIG. 30A—flow cytometry of co-cultured CD105+ cells at passage 0 (P0) using CD31 and αSMA antibodies; FIG. 30B—flow cytometry of pure CD105+ pericytes at passage 4 (P4) using CD31 and CD105 antibodies; FIG. 30C—immunofluorescence of CD105+/CD31− cells (from passage P6) using NG2 (green) and Claponin (red). Nuclei are stained with DAPI (blue). Note that throughout the culture period between 94%-97.5% CD31− pericytes were α-SMA negative (FIG. 30A). All pericytes were CD31−/CD105+ as shown by flow cytometry analysis (FIG. 30B) and stably expressed NG2 and Calponin (FIG. 30C).

FIGS. 31A-B are images depicting tube formation and sprouting on Matrigel. Pericytes form tubular network when seeded on Matrigel with sprouting clusters (arrows) within 12 hours (FIG. 31A). Morphology of pericytes at passage 4 (FIG. 31B). The pericyte cells exhibit high expansion capability, e.g., from 5×10⁵ in passage 0 (P0) to 4×10⁷ in P4 (passage 4).

FIGS. 32A-C are images depicting implants in immuno-deficient NOD/SCID mice. PSC-derived pericytes (from passage 6) were mixed in Matrigel or empty Matrigel were implanted subcutaneously into immune-deficient mice. One week post implantation, implants containing H9.2-derived pericytes are vascularized (FIG. 32A, left) in comparison to empty control Matrigel implant (FIG. 32A, right). Empty implants do not contain blood vessels (FIGS. 32B and 32C) as seen by Hematoxylin and Eosin staining. Original magnifications: ×40, in FIG. 32B; ×100 in FIG. 32C. These results demonstrate that CD105+/CD31− pericyte progenitor cells induce vasculogenesis in vivo.

FIGS. 33A-D are microscopy images depicting pericyte-Matrigel implants in immunodefficient NOD/SCID mice. PSC-derived pericytes (from passage 6) were mixed in Matrigel and implanted subcutaneously into immune-deficient mice. One week post implantation, implants containing H9.2-derived pericytes contain murine blood vessels as seen by Hematoxylin and Eosin staining (FIGS. 33A and 33B). In addition, all PSC-derived pericytes further differentiate into smooth muscle cells, which express α-SMA (red) after implantation (FIGS. 33C and 33D). Nuclear staining by DAPI (blue). Original magnifications: ×100 in FIGS. 33A and 33C; ×200 in FIGS. 33B and 33D. These results demonstrate that CD105+/CD31− pericyte progenitor cells induce vasculogenesis in vivo.

FIGS. 34A-B are microscopy images depicting immunofluorescence analyses of newly formed vasculature within Matrigel implants. Differentiated α-SMA (red) positive PSC-derived pericytes are seen incorporated to newly formed murine blood vessels within Matrigel implant (FIGS. 34A and 34B). Nuclear staining by DAPI appears in blue (FIG. 34A). Original magnifications: ×630.

FIG. 35 is an image depicting tube like formation by pericytes and endothelial cells on Matrigel with pericyte sprouting. Cultured PSC-derived pericytes and endothelial cells were re-mixed post their separate expansion. Note the assembly of the cells to form tube like structures on Matrigel in vitro.

FIGS. 36A-C are microscopy images depicting Hematoxylin and Eosin staining of Matrigel implants of endothelial cells and pericytes. Matrigel was mixed with H9.2-derived pericytes and endothelial cells and implanted subcutaneously into immune-deficient mice. Implants were harvested after one week. Hematoxylin and Eosin staining reveals the presence of erythrocytes containing blood vessels (indicated by arrows) surrounded by Matrigel islands (M) and clusters of PSC-derived pericytes (FIGS. 36A-36C). Original magnifications: ×100 in FIG. 36A; ×200 in FIGS. 36B and 36C.

FIG. 37 is a graph depicting expansion of CD31+ cells in dissociated EBs using various dissociation methods. Single cell suspensions were achieved by incubation of EBs for 20 minutes at 37° C. on shaker with either (1) 0.5% Trypsin/EDTA (Sigma), (2) non-enzymatic solution (Sigma) or (3) 1 mg/ml collagenase B in PBS and 150 U/ml DNAse I (Roche), followed by addition of Trypsin-EDTA (0.05%) for another 5 minutes. To minimize cell aggregation the dissociated EBs were then passed several times through a 20-Gauge needle and filtered through PBS/0.5% FBS pre-washed 0.45 μm cell-strainers (BD Biosciences). Note that the Collagenase B+ DNAse I method revealed a higher dissociation efficiency than the Trypsin method or the non-enzymatic solution-based method. In addition, the viability of the cells dissociated using the Collagenase B+ DNAse I method was 95% as compared to only 50-60% using the other methods (Trypsin or the non-enzymatic solution).

FIG. 38 is a microscopy image of myotubes in culture generated by differentiation of iPSC C3-derived pericytes.

FIGS. 39A-F are dot plots depicting flow cytometry analyses showing the expression of CD73 and CD31 in cells derived from developing EBs. EBs were spontaneously formed (differentiated) from induced pluripotent stem cells (FIGS. 39A-F) and FACS analysis was performed at the indicated days of EBs differentiation: Day 1 (FIG. 39A), day 4 (FIG. 39B), day 10 (FIG. 39C), day 14 (FIG. 39D), day 19 (FIG. 39E), and day 26 (FIG. 39F). The Y axis in each panel represents CD73 expression in the dissociated EBs single cells, and the X axis in each panel represents CD31 expression in the dissociated EBs single cells. H9.2 hESC-derived developing EBs were analyzed similarly and exhibit similar pattern of marker expressions (data not shown).

### DESCRIPTION OF SPECIFIC EMBODIMENTS OF THE INVENTION

The present invention, in some embodiments thereof, relates to isolated pericyte progenitor cells and methods of generating and using same, and more particularly, but not exclusively, to methods of co-derivation two distinguished vasculogenic populations, pericytes and endothelial cells, from pluripotent stem cells and embryoid bodies.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

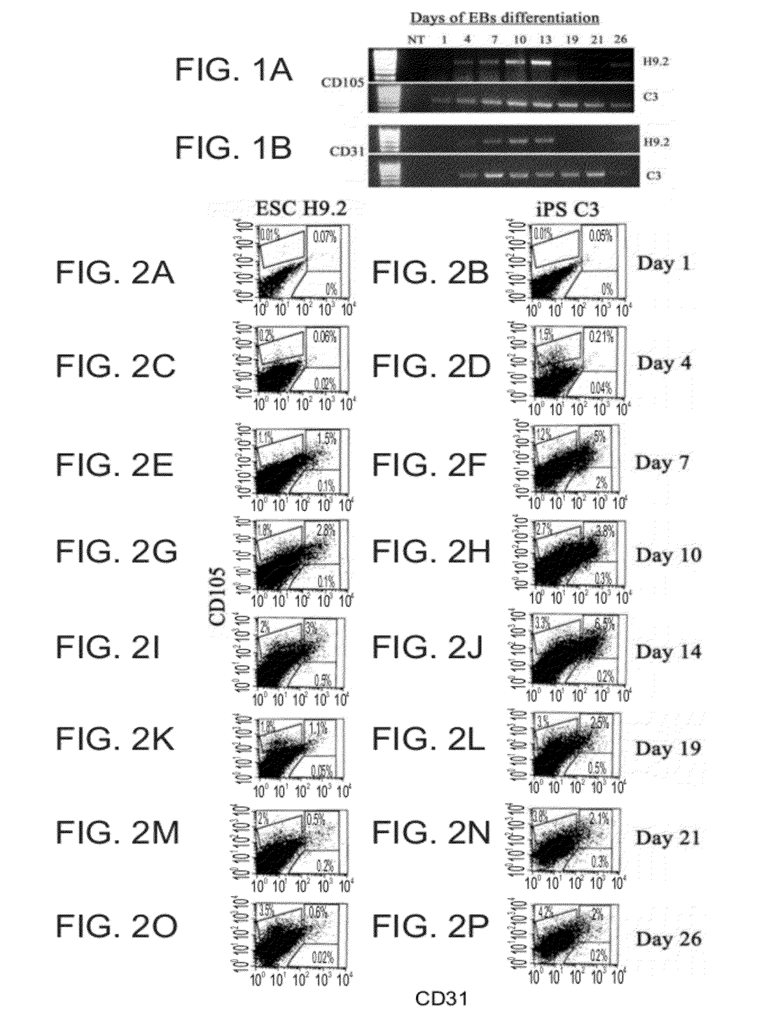

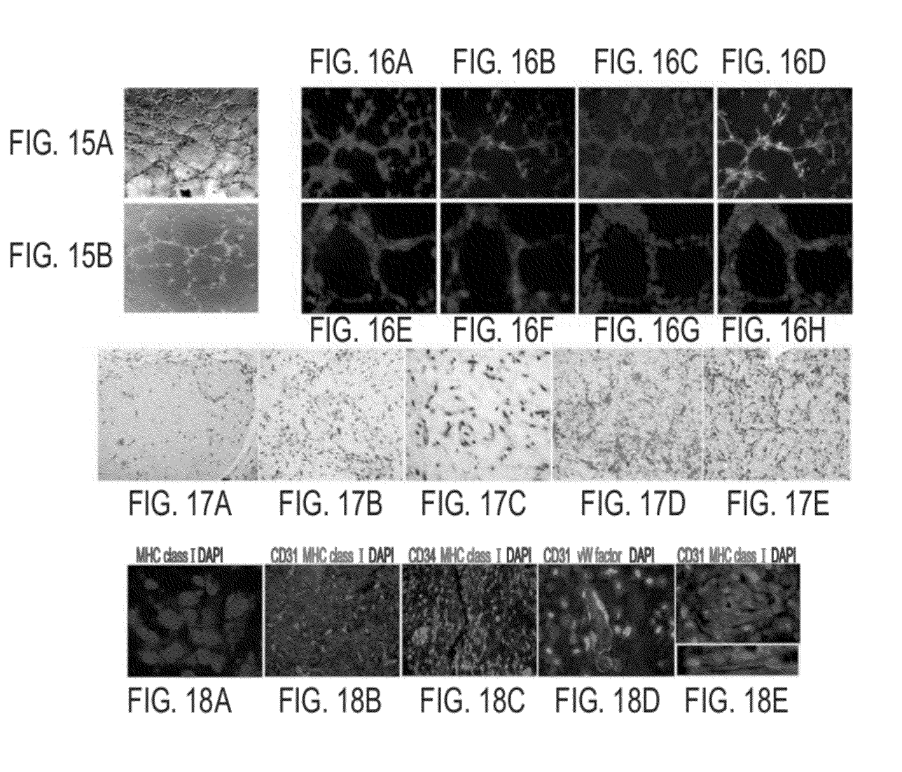

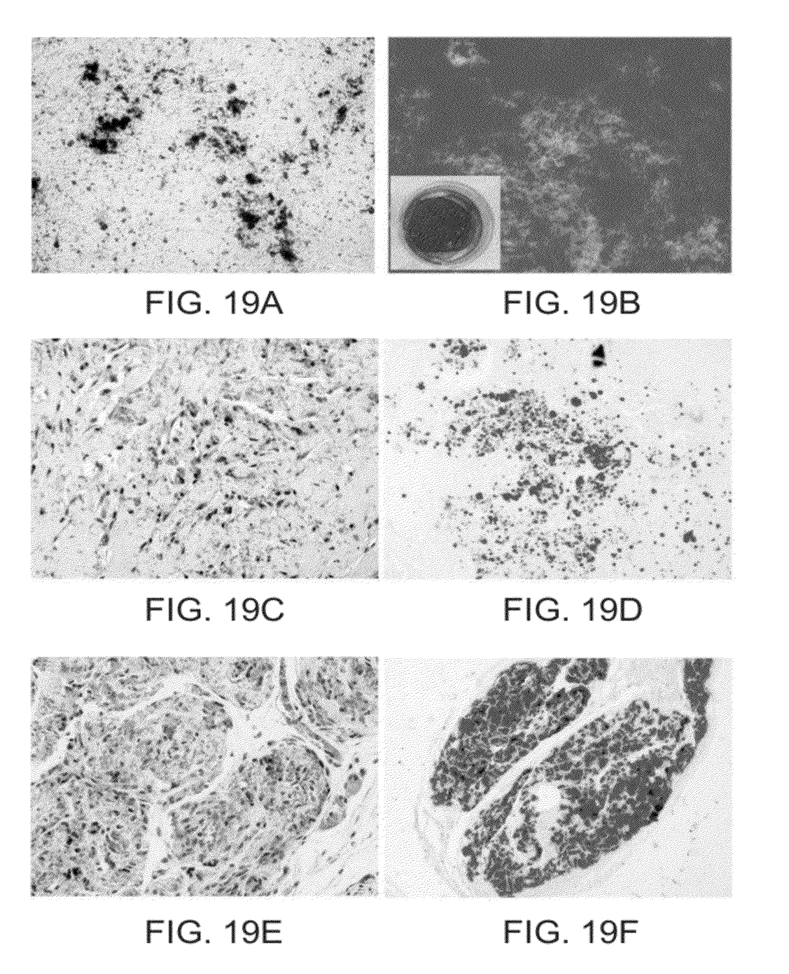

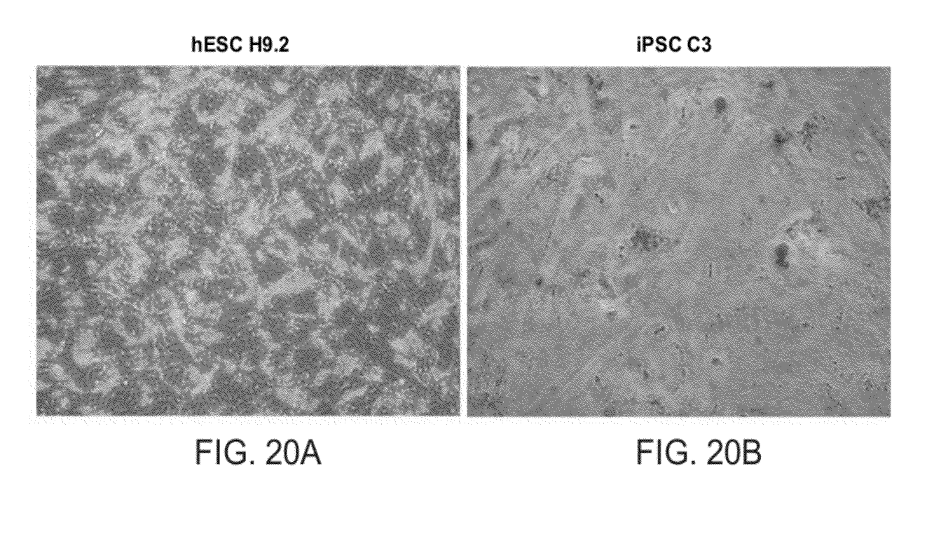

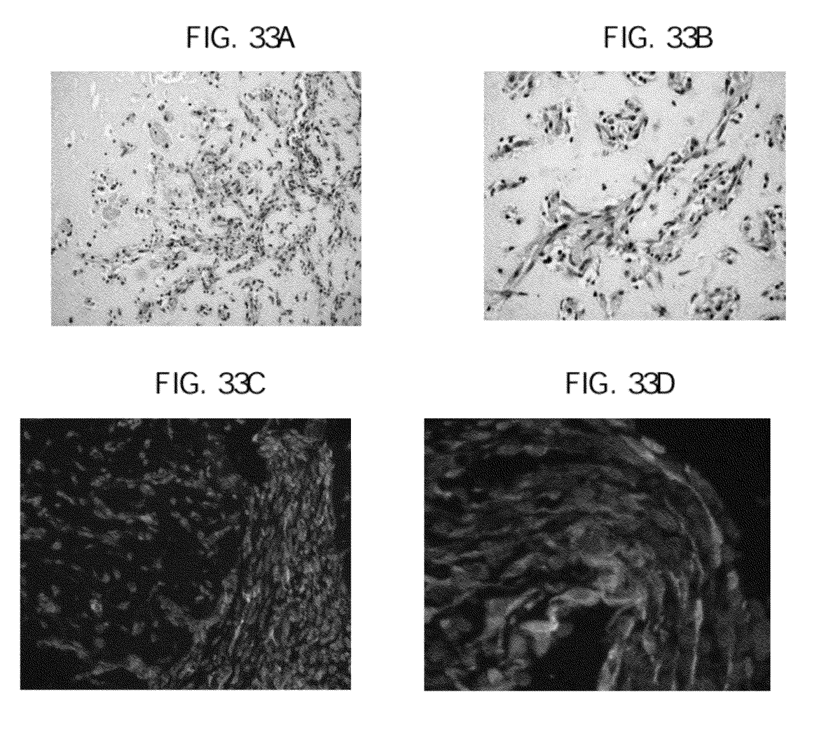

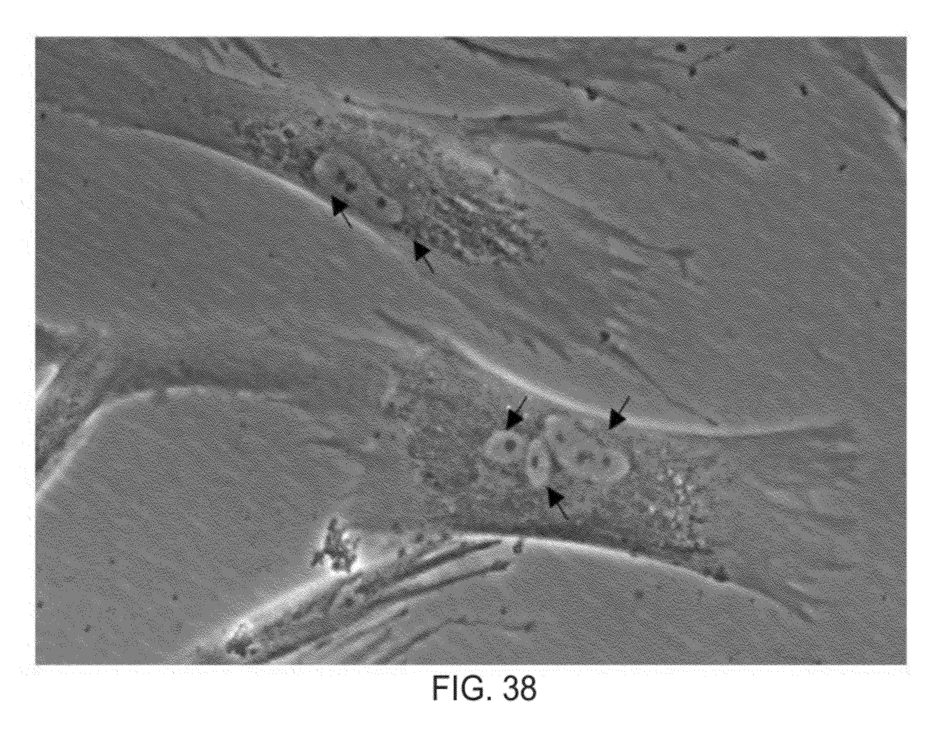

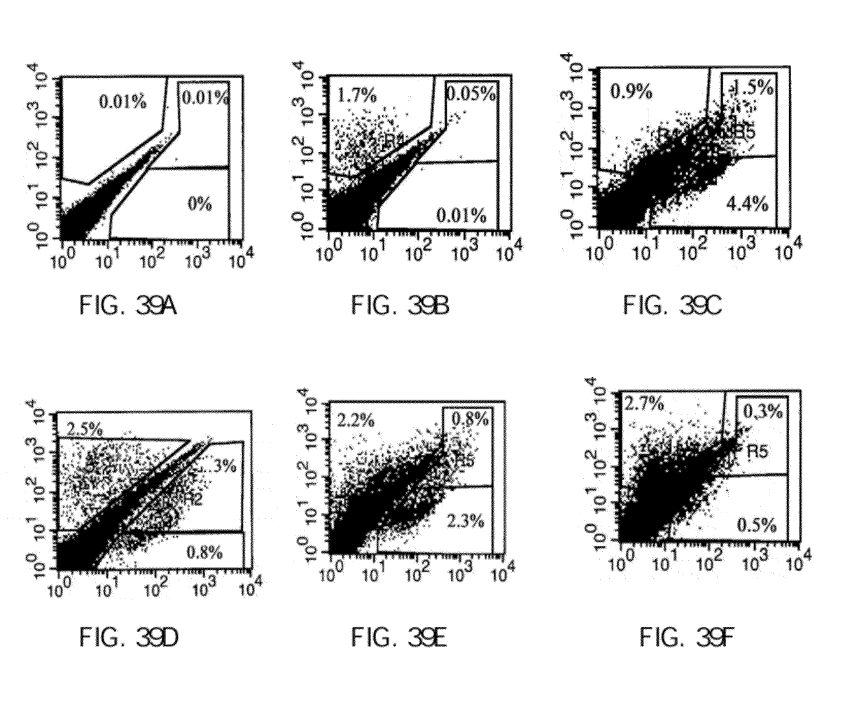

The present inventors have isolated vascular progenitor cells from differentiating embryoid bodies generated from either human embryonic stem cells or induced pluripotent stem cells. Isolated CD105+ cells were subject to co-derivation into pericyte progenitor cells having the CD105+/CD73+/CD31− expression signature, and endothelial progenitor cells having the CD105+/CD73+/CD31+ expression signature (FIGS. 1-3, 21-24, 26, 27, 39; Examples 1, 2, 6 and 7 of the Examples section which follows). As is further described in Examples 2 and 3 of the Examples section which follows (e.g., FIGS. 5-14, Table 2), the isolated pericyte progenitor cells exhibit a unique expression pattern characterized by the CD105+/CD73+CD31−/αSMA−/CD133−/Flk-1− signature. The pericyte progenitor cells were shown capable of assembly into a human vascular network (FIGS. 15-18, 31, 32, 35 and 36, Examples 4 of the Examples section which follows), as well as to differentiate to multiple cell types of the mesenchymal lineage including differentiation into osteoblasts both in vitro and in vivo (FIGS. 19A-F, Example 5 of the Examples section which follows), chondrocytes (Example 5 of the Examples section which follows), adipocytes (FIGS. 20A-B, Example 5 of the Examples section which follows), myoblasts (FIG. 38, Example 5 of the Examples section which follows) and to smooth muscle cells (FIGS. 33C-D, 34A-B, Example 5 of the Examples section which follows).

According to an aspect of some embodiments of the invention, there is provided a method of isolating a pericyte progenitor cell from embryoid bodies. The method is effected by: (a) isolating CD105+, CD73+ and/or CD105+/CD73+ cells from the embryoid bodies, to thereby obtain CD105+, CD73+ and/or CD105+/CD73+ cells, and; (b) culturing the CD105+, CD73+ and/or CD105+/CD73+ cells, thereby isolating the pericyte progenitor cell from the embryoid bodies.

As used herein the phrase “pericyte progenitor cell” refers to an immature pericyte cell (i.e., a more primitive cell in the hierarchy of pericyte differentiation) which is capable of further differentiation into cell type(s) of a mesenchymal lineage. Examples of cells originating from a mesenchymal lineage include, but are not limited to chondrocytes, osteoblasts, adipocytes and myoblasts.

Pericyte cells, also known as Rouget cell, adventitial cell or mural cell, are perivascular cells present in small blood vessels. Pericytes are characterized by the expression of NG2 [chondroitin sulfate proteoglycan 4, official gene symbol: CSPG4, also known as MCSP; MCSPG; MSK16; HMW-MAA; MEL-CSPG] and PDGFR-β [platelet-derived growth factor receptor, beta polypeptide, official gene symbol: PDGFRB, also known as JTK12; PDGFR; CD140B; PDGFR1] (Ugur Ozerdem and William B. Stallcup, Angiogenesis. 2003; 6: 241-249). In addition, while mature pericytes express αSMA (alpha smooth muscle actin; Gene symbol: ACTA2, also known as AAT6; ACTSA), this marker is considered a late marker for differentiated pericytes in rodents and therefore may be poorly expressed in developing angiogenic microvasculature (Ugur Ozerdem and William B. Stallcup, Angiogenesis. 2003; 6: 241-249).

As used herein the phrase “embryoid bodies” (EBs) refers to three dimensional multicellular aggregates of differentiated and undifferentiated cells derivatives of three embryonic germ layers.

Embryoid bodies are formed upon the removal of embryonic stem cells (ESCs) or induced pluripotent stem cells (iPSCs) from feeder layers or feeder cells-free culture systems. ESCs and/or iPSCs removal can be effected using type IV Collagenase treatment for a limited time. Following dissociation from the culturing surface, the cells are transferred to tissue culture plates containing a culture medium supplemented with serum and amino acids.

During the culturing period, EBs are further monitored for their differentiation state. Cell differentiation can be determined upon examination of cell or tissue-specific markers which are known to be indicative of differentiation. For example, EB-derived-differentiated cells may express the neurofilament 68 KD which is a characteristic marker of the ectoderm cell lineage.

The differentiation level of the EB cells can be monitored by following the loss of expression of Oct-4, and the increased expression level of other markers such as α-fetoprotein, NF-68 kDa, α-cardiac and albumin. Methods useful for monitoring the expression level of specific genes are well known in the art and include RT-PCR, semi-quantitative RT-PCR, Northern blot, RNA in situ hybridization, Western blot analysis and immunohistochemistry.

Embryoid bodies can be generated from various primates and mammals such as human, monkeys and rodents (e.g., mouse, rat).

According to some embodiments of the invention, the embryoid bodies are obtained from human embryoid bodies.

According to some embodiments of the invention, the embryoid bodies are obtained by spontaneous differentiation of pluripotent stem cells.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may read on cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO2006/040763) and embryonic germ (EG) cells which are obtained from the genital tissue of a fetus any time during gestation, preferably before 10 weeks of gestation.

The embryonic stem cells of some embodiments of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact ICM by gentle pipetting. The ICM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It will be appreciated that commercially available stem cells can also be used with this aspect of the present invention. Human ES cells can be purchased from the NIH human embryonic stem cells registry (www.escr.nih.gov). Non-limiting examples of commercially available embryonic stem cell lines are BG01, BG02, BG03, BG04, CY12, CY30, CY92, CY10, TE03 and TE32.

In addition, ES cells can be obtained from other species as well, including mouse (Mills and Bradley, 2001), golden hamster [Doetschman et al., 1988, Dev Biol. 127: 224-7], rat [Iannaccone et al., 1994, Dev Biol. 163: 288-92] rabbit [Giles et al. 1993, Mol Reprod Dev. 36: 130-8; Graves & Moreadith, 1993, Mol Reprod Dev. 1993, 36: 424-33], several domestic animal species [Notarianni et al., 1991, J Reprod Fertil Suppl. 43: 255-60; Wheeler 1994, Reprod Fertil Dev. 6: 563-8; Mitalipova et al., 2001, Cloning. 3: 59-67] and non-human primate species (Rhesus monkey and marmoset) [Thomson et al., 1995, Proc Natl Acad Sci USA. 92: 7844-8; Thomson et al., 1996, Biol Reprod. 55: 254-9].

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, Mo., USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

EG cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

Induced pluripotent stem cells (iPS; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as skin) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kfl4 and c-Myc in a somatic stem cell. Thus, iPS cells can be generated by retroviral transduction of somatic cells such as fibroblasts, hepatocytes, gastric epithelial cells with transcription factors such as Oct-3/4, Sox2, c-Myc, and KLF4 [Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); 111 Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872]. Other embryonic-like stem cells can be generated by nuclear transfer to oocytes, fusion with embryonic stem cells or nuclear transfer into zygotes if the recipient cells are arrested in mitosis.

According to some embodiments of the invention, isolating the pericyte progenitor cell is effected using pluripotent stem cells, which are induced to form embryoid bodies prior to isolation of the CD105+, CD73+ and/or CD105+/CD73+ cells.

According to some embodiments of the invention, the embryoid bodies or the pluripotent stem cells are derived from an individual having a normal karyotype according to the species to which the individual belong. For example, for human individuals, a normal karyotype is of 22XY or 22XX chromosomes.

According to some embodiments of the invention, the embryoid bodies or the pluripotent stem cells are derived from a healthy individual.

According to some embodiments of the invention, embryoid bodies or the pluripotent stem cells are derived from an individual having a disease or a pathology, such as a pathology related to an impaired, dysfunction, absence or destroyed vascular tissue. Non-limiting examples include individuals having ischemia, diabetes, diabetic microangiopathy, peripheral arterial disease, cardiovascular diseases, cardiovascular autoimmune diseases, atherosclerosis, vascular injury, vascular repair due to removal of cancerous tissue and the surrounding vasculature, tissue fibrosis, cancer, Alzheimer's disease, chronic lung disease, trauma, injury, cancer, diabetes, blood coagulation related-disorders (e.g., over coagulation).

According to some embodiments of the invention, the embryoid bodies are cultured on defined, xeno-free, feeder-free culturing systems. Such feeder-free systems (e.g., using low attachment culture dishes) can include a culture medium which is serum-free, and/or xeno-free (i.e., devoid of contamination by another species, for example, devoid of animal contamination of human cells) provides a more defined environment for the EBs, which can be controlled, such as to be free of xeno-contaminant and cellular contaminants.

Isolating the CD105+ (cells which express CD105), the CD73+ (cells which express CD73), and/or the CD105+/CD73+ (cells which express both CD105 and CD73 markers) from the embryoid bodies can be performed at any EBs differentiation stage, as long as the EBs include CD105+, CD73+ and/or CD105+/CD73+ cells. As shown in FIGS. 1A-B, 2A-P, 3A-D, 39A-F and described in Examples 1 and 6 of the Examples section which follows, the fraction of CD105+ or CD73+ cells significantly increases between day 4 and 7, and further between day 7 to 10 and 10 to 14 of EBs differentiation. In addition, the fraction of CD105+/CD31− cells increases between day 14 and 26 of EBs differentiation.

According to some embodiments of the invention isolating CD105+, CD73+ and/or CD105+/CD73+ cells is performed between about day 4 to about day 26 of EBs differentiation, e.g., between about day 7 to about day 26 of EBs differentiation, e.g., between about day 10 to about day 26 of EBs differentiation, e.g., between about day 14 to about day 26 of differentiation of the embryoid bodies.

It should be noted that the first day of EBs differentiation is considered about 24 hours after the pluripotent stem cells were allowed to differentiate in vitro by removing the pluripotent stem cells from their undifferentiation culture conditions, such as by removing them from feeder layers or from their feeder-free culture systems (e.g., matrix such as an extracellular matrix).

Isolating the CD105+, CD73+ and/or CD105+/CD73+ cells from the embryoid bodies can be performed by any immunological based method which results in the physical isolation of cells having a specific cell surface marker using an antibody or an antibody fragment which specifically recognizes the marker. Examples include, but are not limited to isolation by fluorescence-activated cell sorting using the specific antibodies, magnetic beads coated by the specific antibodies, and columns coated by the specific antibodies.

CD105, also known as endoglin (gene symbol ENG) is a homodimeric transmembrane protein, a major glycoprotein of the vascular endothelium. Endoglin is a component of the transforming growth factor beta receptor complex and it binds TGFB1 and TGFB3 with high affinity. There are two known variants of endoglin: isoform 1 (GenBank Accession No. NP_{—}001108225.1; SEQ ID NO:17) and isoform 2 (GenBank Accession No. NP_{—}000109.1; SEQ ID NO:18).

According to some embodiments of the invention, isolating the CD105+ and/or the CD105+/CD73+ cells is effected using an anti CD105 antibody.

Suitable CD105 antibodies which can be used to isolate the CD105+ or CD105+/CD73+ cells from the EBs include R-Phycoerythrin (PE)-conjugated anti-CD105 (eBioscience), Fluorescein isothiocyanate (FITC)-conjugated anti-CD105 (ABCAM), APC-conjugated anti-CD105 (eBioscience).

CD73, Ecto-5-prime-nucleotidase, also known as NT; eN; NTS; NTE; eNT; E5NT; NT5E (EC 3.1.3.5; GenBank Accession No. NP_{—}002517.1; SEQ ID NO:19), catalyzes the conversion at neutral pH of purine 5-prime mononucleotides to nucleosides, the preferred substrate being AMP. The enzyme consists of a dimer of 2 identical 70-kD subunits bound by a glycosyl phosphatidyl inositol linkage to the external face of the plasma membrane.

According to some embodiments of the invention, isolating the CD73+ and/or CD105+/CD73+ cells is effected using a CD73 antibody.

Suitable CD73 antibodies which can be used to isolate the CD73+ or CD73+/CD105+ cells from the EBs include PE-conjugated anti-CD73 (BD Pharmingen), FITC-conjugated anti-CD73 (eBioscience), APC conjugated anti-CD73 (eBioscience).

For isolation using fluorescence-activated cell sorting, the cells are labeled with a fluorescent antibody (e.g., PE-conjugated anti CD105 antibody, or PE-conjugated anti CD73 antibody) and then inserted into a cell sorter (e.g., FACS Aria sorter).

For isolation using magnetic beads, the cells are labeled with a magnetic bead conjugated antibody anti CD105 antibody (Miltenyi Biotec) or anti CD73 antibody; alternatively, the cells can be labeled with a non-conjugated antibody and followed by incubation with a match isotype bead conjugated secondary antibody (anti mouse IgG1 bead conjugated). Isolation is performed using magnetic cell separation column such as MAX (Miltenyi Biotec).

It should be noted that prior to isolation of CD105+, CD73+ and/or CD105+/CD73+ cells, the EBs are dissociated to separate the cell clumps and cell aggregates into single cells. The dissociation is performed under conditions which enable separation of cell aggregates/clumps while preserving the viability of the separated cells of the dissociated EBs.

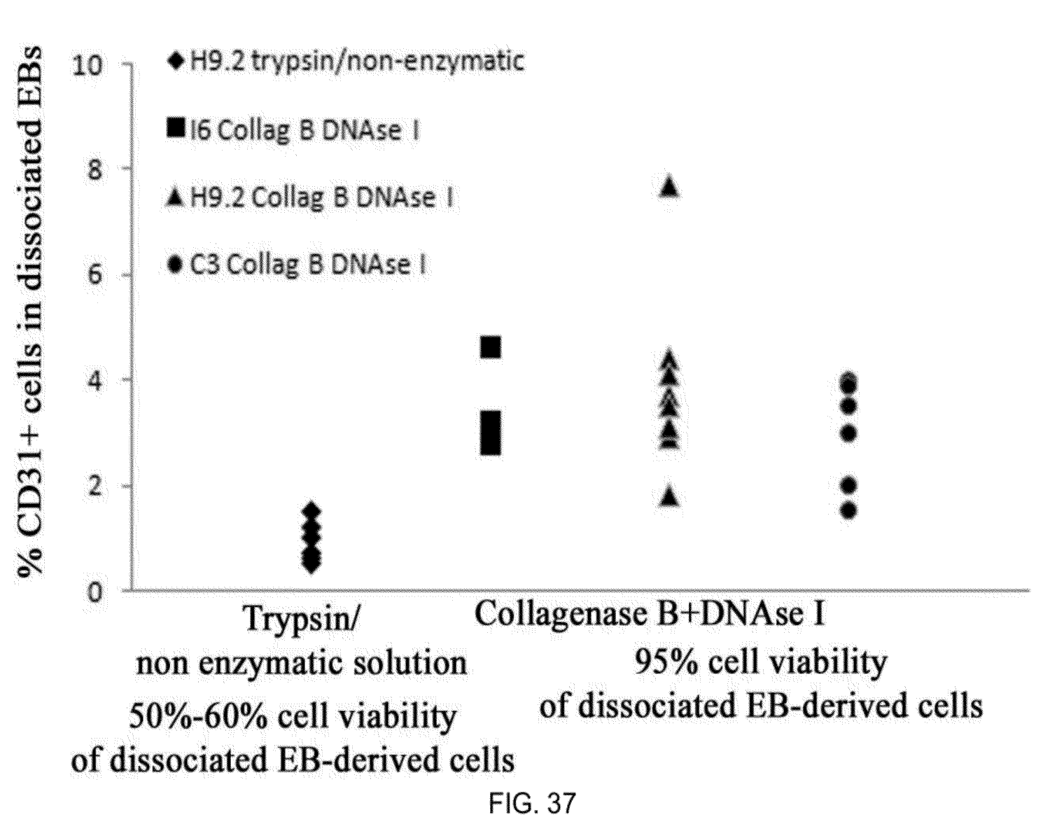

As shown in FIG. 37 and described in Example 1 of the Examples section which follows, incubation of EBs in a solution which include Collagenase B and DNAse I, resulted in up to 2-10 fold increase in the apparent percentage of dissociated CD105+CD31+ cells and significantly increased the yield of viable cells to 95%.

According to some embodiments of the invention, the dissociation of EBs is performed by treatment with Collagenase and DNAse I. The Collagenase can be Collagenase B (e.g., available from Roche, catalogue number 11 088 807 001) used in a concentration in the range of about 0.1-5 mg/ml, e.g., about 0.5-3 mg/ml, e.g., about 0.8-2 mg/ml, e.g., about 0.8-1.5 mg/ml, e.g., about 1 mg/ml Collagenase B. The DNAse I (e.g., available from Roche, catalogue number 2139) can be used in a concentration of about 10-500 U/ml, e.g., about 50-350 U/ml, e.g., about 100-200 U/ml, e.g., about 150 U/ml DNAse I.

Incubation with the Collagenase and DNAse I solution can be performed while shaking the vessel containing the EBs, for an incubation time which may vary between about 5-30 minutes, e.g., between about 10-25 minutes, e.g., between about 15-20 minutes. To increase efficiency, the dissociation can be performed at about 37° C. while shaking. Trypsin may be added for a limited time (e.g., about 5 minutes) and concentration (e.g., a solution of about 0.05%). However, measures are taken to avoid cell damage, and the dissociation conditions can be adjusted according to the source or origin of the EBs.

To minimize cell aggregation the dissociated EBs can be further passed several times through a 20-Gauge needle and filtered through a cell-strainer (e.g., PBS/0.5% fetal bovine serum (FBS) pre-washed 0.45 cell-strainers (BD Biosciences).

It should be noted that while the fraction of CD105+ cells in differentiating EBs of days about 4-26 is about 1-4% (FIGS. 2A-P), following the isolation step the majority of the cells are CD105⁺.

According to some embodiments of the invention, the isolation step with the anti-CD105 antibody (or antibodies) results in a population of cells which comprises at least about 85% of CD105+ or CD105+/CD73+ cells, e.g., at least about 86%, 87%, 88%, 89%, 90%, 91%, 92%, 93%, 94%, 95%, 96%, 97%, 98%, 99% or 100% of cells which are CD 105+ or CD105+/CD73+.

According to some embodiments of the invention, the isolation step with the anti-CD73 antibody (or antibodies) results in a population of cells which comprises at least about 85% of CD73+ or CD105+/CD73+ cells, e.g., at least about 86%, 87%, 88%, 89%, 90%, 91%, 92%, 93%, 94%, 95%, 96%, 97%, 98%, 99% or 100% of cells which are CD73+ or CD105+/CD73+.

As described in the Examples section which follows, the present inventors demonstrated the isolation of pericyte progenitor cells by enriching the CD105+/CD31-cells (i.e., cells which express CD105 and which do not express CD31) in the isolated population of cells.

Thus, according to some embodiments of the invention, the method further comprising enriching the cells for CD105+/CD31−, CD73+/CD31− and/or CD105+/CD73+/CD31− cells.

As used herein the phrase “enriching . . . cells” refers to increasing the percentage of cells characterized by a specific marker expression signature in a heterogenous population of cells. Thus, while the heterogenous population of cells isolated from EBs includes CD105+, CD73+ and/or CD105+/CD73+ cells which are either CD31+ or CD31−, the enrichment step results in a majority of cells which are CD31−.

According to some embodiments of the invention, the enrichment step results in a population of cells which comprises at least about 90%, 91%, 92%, 93%, 94%, 95%, 96%, 97%, 98% 99% or 100% of cells which are CD105+/CD31−, CD73+/CD31− and/or CD105+/CD73+/CD31−.

According to some embodiments of the invention, enriching the cells is effected prior to culturing the isolated CD105+, CD73+ and/or CD105+/CD73+ cells from the EBs.

According to some embodiments of the invention, enriching is performed by depleting CD31+ cells from the CD105+, CD73+ and/or CD105+/CD73+ cells.

The phrase “depleting . . . cells” refers to decreasing the percentage of cells characterized by a specific marker expression signature in a heterogenous population of cells and/or removing cells characterized by the specific marker expression signature from a heterogenous population of cells, such that the population of cells is devoid of the depleted cells.

For example, if a heterogenous population of cells comprises both CD31+ and CD31− cells, then depleting the CD31+ cells results in significantly decreasing the percentage of CD31+ from the heterogenous population and optionally, completely removing the CD31+ cells from the heterogenous population in order to obtain a population which is characterized by the CD31− expression signature.

According to some embodiments of the invention, the depletion step results in a population of cells which comprises no more than 20%, e.g., no more than 19%, 18%, 17%, 16%, 15%, 14%, 13%, 12%, 11%, 10%, 9%, 8%, 7%, 6%, 5%, 4%, 3%, 2%, 1% or 0% of cells which are CD105+/CD31+, CD73+/CD31+ and/or CD105+/CD73+/CD31+.

Depleting CD31+ cells can be performed by fluorescence-activated cell sorting or using magnetic beads as described above, except that the cells which are CD31+ are removed from the cell population and the cells which are CD31− are retrieved (isolated).

As described in the Examples section which follows, when CD105+ cells were allowed to grow in culture for about 1-2 passages the pericyte progenitor cells dominated the culture without an active enrichment step.

According to some embodiments of the invention, the method further comprising passaging the CD105+, CD73+ and/or CD105+/CD73+ cells for at least 1-2 passages to thereby expand a population of pericyte progenitor cells.

Culturing of the isolated CD105+, CD73+ and/or CD105+/CD73+ cells can be performed in a two-dimensional culture system by seeding the cells on a surface of a culture dish (e.g., a plastic dish, a flask).

It should be noted that for passage 0 and/or passages 1-2, the CD105+, CD73+ and/or CD105+/CD73+ cells can be cultured on coated plates/flasks to increase adhesiveness of the cells. However, once the pericyte progenitor cells are isolated and/or dominate the culture, the cells can be cultured without further coating of tissue culture dishes (plates/flasks).

As mentioned, for passage 0, and optionally passages 1-2, the culture dish can be coated with an extracellular matrix, preferably a synthetic or xeno-free extracellular matrix.

Non-limiting examples of matrices which can be used include foreskin matrix, laminin matrix, fibronectin matrix, proteoglycan matrix, entactin matrix, heparan sulfate matrix, collagen matrix and the like, alone or in various combinations thereof.

According to some embodiments of the invention the matrix is xeno-free.

The term “xeno” is a prefix based on the Greek word “Xenos”, i.e., a stranger. As used herein the phrase “xeno-free” refers to being devoid of any components which are derived from a xenos (i.e., not the same, a foreigner) species. Such components can be contaminants such as pathogens associated with (e.g., infecting) the xeno species, cellular components of the xeno species or a-cellular components (e.g., fluid) of the xeno species.

In cases where complete animal-free culturing conditions are desired, the matrix is preferably derived from a human source or synthesized using recombinant techniques such as described hereinabove. Such matrices include, for example, human-derived fibronectin, recombinant fibronectin, human-derived laminin, foreskin fibroblast matrix or a synthetic fibronectin matrix. Human derived fibronectin can be from plasma fibronectin or cellular fibronectin, both of which can be obtained from Sigma, St. Louis, Mo., USA. Human derived laminin and foreskin fibroblast matrix can be obtained from Sigma, St. Louis, Mo., USA. A synthetic fibronectin matrix can be obtained from Sigma, St. Louis, Mo., USA.

According to some embodiments of the invention, the matrix is a human fibronectin matrix.

The medium used to culture and/or expand the CD105+, CD73+ and/or CD105+/CD73+ cells can be an endothelial cell growth medium, such as an M-199 based culture medium (available from Biological Industries, Israel). The medium can be supplemented with serum (e.g., human serum, bovine serum, or serum replacement) and additional additives such as an endothelial cell growth supplement (ECGS) (available from Zotal Biological & Instrumentation, Israel). A non-limiting example of an endothelial cell medium which can be used include the M-199 medium containing 20% defined-fetal bovine serum (FBS) (HyClone, Utah, USA), 1% Pen-Strep, 1% 1-glutamine, 1 mM HEPES (Biological Industries), 20 U/ml heparin (Sigma-Aldrich) and 50 μg/ml endothelial cell growth supplement (ECGS) (Zotal). During culturing the medium is usually replaced with a fresh medium every about 2-3 days.

According to some embodiments of the invention, the medium is xeno-free and comprises either human serum or a xeno-free serum replacement. For example, a xeno-free serum replacement can include a combination of insulin, transferrin and selenium. Additionally or alternatively, a xeno-free serum replacement can include human or recombinantly produced albumin, transferrin and insulin.

Passaging can be performed by dissociating cells from the wall of the culture vessel using e.g., type IV collagenase (at a concentration of 0.1% for 20-60 minutes) followed by trypsinization (using 0.25% trypsin for 2-5 minutes), counting the single cells and splitting the cells to 2-3, (i.e., a splitting ratio of 1:2 or 1:3) in order to preserve the same cell density of their initial seeding (e.g., about 5×10⁵-1×10⁶ cells per 15 ml in T75 flasks). According to some embodiments of the invention, the cell culture is subjected to culture passaging every 2-8 days, e.g., culture passaging occurs every 3-8 days, e.g., every 4 days.

Culturing can be performed for several passages, e.g., from 1-9 passages, e.g., 1-2,1-3, 1-4, 1-5, 1-6, 1-7, 1-8, 7-9 passages, or until senescence.

As used herein the term “senescence” refers to the stage in which the cells lose their ability to divide.

According to some embodiments of the invention, even in the absence of an active enrichment step, following about 1-2 passages at least 96%, 97%, 98%, 99% or 100% of the cells having a signature of CD105+/CD73+/CD31−.

It should be noted that pericyte progenitor cells can be isolated from EBs in either way, i.e., with or without the active enrichment of CD31− cells.

The CD105+/CD73+/CD31− cells can be further cultured in order to expand the pericyte progenitor cell population.

As shown in FIGS. 12C-D and described in Example 3 of the Examples section which follows, the cells isolated from EBs according to the method of some embodiments of the invention exhibit pericyte characteristics in term of expression of NG2 and PDGFR-β.

According to some embodiments of the invention, while in culture (e.g., in a two dimensional culture dish), the pericyte progenitor cells are substantially free of contact inhibition.

According to some embodiments of the invention, while in culture (e.g., in a two dimensional culture dish), the pericyte progenitor cells adopt a hill and valley morphology.

As shown in FIGS. 12C-D and 13, the majority of the isolated cells do not express αSMA, a late marker of mature pericytes.

Thus, according to an aspect of some embodiments of the invention, there is provided an isolated population of cells which comprises at least 80%, 81%, 82%, 83%, 84%, 85%, 86%, 87%, 88%, 89%, 90%, 91%, 92%, 93%, 94%, 95%, 96%, 97%, 98%, 99%, e.g., 100% of pericyte progenitor cells having the CD105+/CD31−/αSMA−, a CD73+/CD31−/αSMA− or a CD105+/CD73+/CD31−/αSMA− signature.

According to some embodiments of the invention, at least about 85%, 86%, 87%, 88%, 89%, 90%, 91%, 92%, 93%, 94%, 95%, 96%, 97%, 98%, 99%, e.g., 100% of the cells isolated by the method of some embodiments of the invention having an expression marker signature of CD105+/αSMA− and/or CD73+/αSMA−.

As shown in FIGS. 2M-N, all of the isolated pericyte progenitor cells do not express CD31.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/CD31− and/or CD73+/CD31−.

According to some embodiments of the invention, at least about 85%, e.g., at least about 86%, 87%, 88%, 89%, 90%, 91%, 92%, 93%, 94%, 95%, 96%, 97% or more of the cells isolated by the method of some embodiments of the invention having an expression marker signature of CD105+/CD31−/αSMA− and/or CD73+/CD31−/αSMA−.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/CD133− and/or CD73+/CD133−.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/NG2+ and/or CD73+/NG2+.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/CD31−/CD133− and/or CD73+/CD31−/CD133− cells.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/CD31−/CD133−/NG2+ and/or CD73+/CD31−/CD133−/NG2+ cells.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/CD146+ and/or CD73+/CD146+.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/CD90+ and/or CD73+/CD90+.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/CD31−/αSMA−/CD146+/CD90+ and/or CD73+/CD31−/αSMA−/CD146+/CD90+.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/CD31−/CD133− and/or CD73+/CD31−/CD133− cells.

According to some embodiments of the invention, at least about 90%, e.g., at least about 91%, 92%, 93%, 94%, 95%, 96%, 97%, 98%, 99% or 100% of the cells isolated by the method of some embodiments of the invention having an expression marker signature of CD105+/CD31−/CD133−/NG2+ and/or CD73+/CD31−/CD133−/NG2+ cells.

According to some embodiments of the invention, after being passaged for about 7-9 passages the population of cells generated by the method of some embodiments of the invention maintains the expression signature of the isolated cells before culturing and/or before passaging. For example, after being passaged for about 1-9 passages, until senescence or during senescence, the population of cells comprises at least about 85%, e.g., at least about 86%, 87%, 88%, 89%, 90%, 91%, 92%, 93%, 94%, 95%, 96%, 97% or more of cells having an expression marker signature of CD105+/CD31−/αSMA− and/or CD73+/CD31−/αSMA−.

According to some embodiments of the invention, after being passaged for about 1-9 passages, until senescence or during senescence, the population of cells comprises at least about 86%, 87%, 88%, 89%, 90%, 91%, 92%, 93%, 94%, 95%, 96%, 97% or more of cells having a CD105+/CD73+/CD31−/αSMA−/CD133−/Flk−/NG2+/CD146+/CD90+/Tie-1+/Tie-2+ signature.

Thus, according to an aspect of some embodiments of the invention, there is provided an isolated pericyte progenitor cell.

As used herein the phrase “isolated” refers to being at least partially separated from the natural environment e.g., the embryoid bodies or from non-pericyte progenitor cells which are present in the EBs.

It should be noted that the term “cell” may also read on a plurality of cells (e.g., a cell line), wherein all of the cells have at least about the same expression marker signature as that of the single cell.

According to some embodiments of the invention, the isolated pericyte progenitor cell is separated from other cells of the EBs, such as cells having a different expression marker signature than that of the isolated pericyte progenitor cell.

According to some embodiments of the invention, the isolated pericyte progenitor cell is separated from cells having an expression marker signature of CD31−/CD105− or CD105+/CD31−/Flk-1+.

According to some embodiments of the invention, when the isolated pericyte progenitor cell is comprised in a plurality of cells, the plurality of cells is devoid of CD31−/CD105− cells and/or being devoid of CD105+/CD31−/Flk-1+ cells.

According to some embodiments of the invention, the isolated pericyte progenitor cell having a CD105+/CD73+/CD31−/αSMA− signature.

According to some embodiments of the invention, the isolated pericyte progenitor cell having a CD105+/CD31−/αSMA−/CD133−, a CD73+/CD31−/αSMA−/CD133− or a CD105+/CD73+CD31−/αSMA−/CD133− signature.

According to some embodiments of the invention, the isolated pericyte progenitor cell having a CD105+/CD31−/αSMA−/CD133−/Flk-1−, a CD73+/CD31−/αSMA−/CD133−/Flk-1− or a CD105+/CD73+CD31−/αSMA−/CD133−/Flk-1− signature.

According to some embodiments of the invention, the pericyte progenitor cell having a CD105+/CD73+/CD31−/αSMA−/CD133− signature.

According to some embodiments of the invention, the pericyte progenitor cell having a CD105+/CD73+/CD31−/αSMA−/Flk1− or CD105+/CD73+/CD31−/αSMA−/CD133−/Flk1− signature.

According to some embodiments of the invention, the pericyte progenitor has an expression marker signature of NG2+/αSMA−.

According to some embodiments of the invention, the pericyte progenitor cell having a CD105+/CD73+/CD31−/αSMA−/NG2+, CD105+/CD73+/CD31−/αSMA−/CD133−/NG2+ or CD105+/CD73+/CD31−/αSMA−/CD133−/Flk1−/NG2+ signature.

According to some embodiments of the invention, the pericyte progenitor cell is CD146+. Such a pericyte cell can have an expression marker signature of CD105+/CD73+/CD31−/αSMA−/Flk1−/CD146+, CD105+/CD73+/CD31−/αSMA−/Flk1−/CD146+, CD105+/CD73+/CD31−/αSMA−/NG2+/CD146+, CD105+/CD73+/CD31−/αSMA−/CD133−/NG2+/CD146+, or CD105+/CD73+/CD31−/αSMA−/CD133−/Flk1−/NG2+/CD146+ signature.

According to some embodiments of the invention, the pericyte progenitor cell is CD90+. Such a pericyte cell can have an expression marker signature of CD105+/CD73+/CD31−/αSMA−/Flk1−/CD90+, CD105+/CD73+/CD31−/αSMA−/CD133−/Flk1−/CD90+, CD105+/CD73+/CD31−/αSMA−/NG2+/CD90+, CD105+/CD73+/CD31−/αSMA−/CD133−/NG2+/CD90+, CD105+/CD73+/CD31−/αSMA−/CD133−/Flk1−/NG2+/CD90+, CD105+/CD73+/CD31−/αSMA−/Flk1−/CD146+/CD90+, CD105+/CD73+/CD31−/αSMA−/Flk1−/CD146+/CD90+, CD105+/CD73+/CD31−/αSMA−/NG2+/CD146+/CD90+, CD105+/CD73+/CD31−/αSMA−/CD133−/NG2+/CD146+/CD90+, or CD105+/CD73+/CD31−/αSMA−/CD133−/Flk1−/NG2+/CD146+/CD90+.
According to some embodiments of the invention, the pericyte progenitor cell is Tie-1+/Tie-2+. Such a pericyte cell can have an expression marker signature of CD105+/CD73+CD31−/αSMA−/CD133−/Flk-1−/Tie-1+/Tie-2+, CD105+/CD73+/CD31−/αSMA−/CD133−/Tie-1+/Tie-2+, CD105+/CD73+/CD31−/αSMA−/Flk1−/Tie-1+/Tie-2+, CD105+/CD73+/CD31−/αSMA−/CD133−/Flk1−/Tie-1+/Tie-2+, CD105+/CD73+/CD31−/αSMA−/NG2+/Tie-1+/Tie-2+, CD105+/CD73+/CD31−/αSMA−/CD133−/NG2+/Tie-1+/Tie-2+, CD105+/CD73+/CD31−/αSMA−/CD133−/Flk1−/NG2+/Tie-1+/Tie-2+, CD105+/CD73+/CD31−/αSMA−/Flk1−/CD146+/Tie-1+/Tie-2+, CD105+/CD73+/CD31−/αSMA−/Flk1−/CD146+/Tie-1+/Tie-2+, CD105+/CD73+/CD31−/αSMA−/NG2+/CD146+/Tie-1+/Tie-2+, CD105+/CD73+/CD31−/αSMA−/CD133−/NG2+/CD146+/Tie-1+/Tie-2+, CD105+/CD73+/CD31−/αSMA−/CD133−/Flk1−/NG2+/CD146+/Tie-1+/Tie-2+, CD105+/CD73+/CD31−/αSMA−/Flk1−/CD90+/Tie-1+/Tie-2+, CD105+/CD73+/CD31−/αSMA−/CD133−/Flk1−/CD90+/Tie-1+/Tie-2+, CD105+/CD73+/CD31−/αSMA−/NG2+/CD90+/Tie-1+/Tie-2+, CD105+/CD73+/CD31−/αSMA−/CD133−/NG2+/CD90+/Tie-1+/Tie-2+, CD105+/CD73+/CD31−/αSMA−/CD133−/Flk1−/NG2+/CD90+/Tie-1+/Tie-2+, CD105+/CD73+/CD31−/αSMA−/Flk1−/CD146+/CD90+/Tie-1+/Tie-2+, CD105+/CD73+/CD31−/αSMA−/Flk1−/CD146+/CD90+/Tie-1+/Tie-2+, CD105+/CD73+/CD31−/αSMA−/NG2+/CD146+/CD90+/Tie-1+/Tie-2+, CD105+/CD73+/CD31−/αSMA−/CD133−/NG2+/CD146+/CD90+/Tie-1+/Tie-2+, CD105+/CD73+/CD31−/αSMA−/CD133−/Flk1−/NG2+/CD146+/CD90+/Tie-1+/Tie-2+.

According to some embodiments of the invention, the pericyte progenitor cell is capable of differentiation into at least two cell lineages of the cell lineages selected from the group consisting of osteoblasts, chondrocytes, myobloasts, smooth muscle cells and apipocytes.

For example, the pericyte progenitor cells were capable of differentiation into osteoblasts (FIGS. 19A-B and Example 5 of the Examples section which follows), adipocytes (FIGS. 20A-B and Example 5 of the Examples section which follows), chondrocytes (Example 5 of the Examples section which follows) myoblasts (FIGS. 33A-D, 38 and Example 5 of the Examples section which follows) and smooth muscle cells (FIGS. 33C-D, 34A-B, Example 5 of the Examples section which follows).

According to some embodiments of the invention, the pericyte progenitor cell is capable of differentiation into at least three, four or five cell lineages of the cell lineages selected from the group consisting of osteoblasts, chondrocytes, myobloasts, smooth muscle cells and apipocytes.

According to some embodiments of the invention, the ability of the pericyte progenitor cell to differentiate into at least two cell lineages of the cell lineages selected from the group consisting of osteoblasts, chondrocytes, myobloasts, smooth muscle cells and apipocytes is maintained at any passage in culture from passage 1 to senescence.

According to some embodiments of the invention, the pericyte progenitor is not genetically modified to express an exogenous gene such as a reporter gene (e.g., green fluorescence protein, GFP).

According to an aspect of some embodiments of the invention, there is provided a cell culture comprising a culture medium and the isolated pericyte progenitor cell of some embodiments of the invention, or the isolated population of cells of some embodiments of the invention.

The medium can be any liquid medium suitable for culturing the pericyte progenitor cells and maintaining them in an undifferentiated state (i.e., capable of multipotent differentiation). Non-limiting examples of such a medium include, but are not limited to an M-199 based culture medium (available from Biological Industries, Israel, Catalogue No. 01-085-1A). The medium can be supplemented with serum (e.g., human serum, bovine serum, or serum replacement) and additional additives such as an endothelial cell growth supplement (ECGS) (available from Zotal Biological & Instrumentation, Israel). A non-limiting example of an endothelial cell medium which can be used include the M-199 medium containing 20% defined-fetal bovine serum (FBS) (HyClone, Utah, USA), 1% Pen-Strep, 1% 1-glutamine, 1 mM HEPES (Biological Industries) 20 U/ml heparin (Sigma-Aldrich) and 50 μg/ml endothelial cell growth supplement (ECGS) (Zotal).

According to an aspect of some embodiments of the invention, there is provided a method of generating osteoblast cells (osteogenic differentiation). The method is effected by culturing the isolated pericyte progenitor cell of some embodiments of the invention, or the isolated population of cells of some embodiments of the invention in an osteogenic medium such as a culture medium which comprises β-glycerol-phosphate, Dexamethasone and ascorbic acid, thereby generating the osteoblast cells.

For example, for osteogenic differentiation, the pericyte progenitor cells are grown in the presence of a culture medium containing 10 mM β-glycerol-phosphate and 0.1 μM Dexamethasone in GMEM BHK-21 medium (which comprises ascorbic acid) (Gibco) containing 10% FBS for 4 weeks, with media changes twice a week. Cell cultures can be assayed for mineral content by Alizarin red staining.

Alternatively, for osteogenic differentiation, the pericyte progenitor cells are grown in the presence of a culture medium containing alpha-MEM (Biological Industries, Kibbutz Beit Haemek, Israel) supplemented with 15% FBS (selected lots, Hyclone), 50 μg/ml ascorbic acid (Sigma, St Louis, Mo., USA), 10⁻⁷ M dexamethasone (Sigma, St Louis, Mo., USA) and 10 mM beta-glycerophosphate (inorganic phosphate), and let become over-confluent for period of at least 10 days before mineralization appears.

According to some embodiments of the invention, the osteoblast cells are characterized by mineral deposits and massive calcium content.

According to an aspect of some embodiments of the invention, there is provided a method of generating adipocyte cells, comprising culturing the isolated pericyte progenitor cell of some embodiments of the invention, or the isolated population of cells of some embodiments of the invention in an adipogenic medium such as a culture medium which comprises IBMX (3-isobutyl-1-methylxanthine), Dexamethasone and insulin, thereby generating the adipocyte cells.

For example, for adipogenic differentiation the pericyte progenitor cells (e.g., seeded at a concentration of 2×10⁵ cells/cm²) are grown for about 4 weeks in the presence of 0.5 mM IBMX (3-isobutyl-1-methylxanthine), 10 μg/ml Insulin, 10⁻⁶ M Dexamethasone, and 0.1 mM Indomethacin in DMEM/F12 medium (Biological Industries, Biet HaEmek, Israel) containing 10% FBS.

According to some embodiments of the invention, the adipocyte cells are characterized by accumulation of lipid-rich vacuoles which are positive for Oil red staining.

According to an aspect of some embodiments of the invention, there is provided a method of generating chondrocyte cells, comprising culturing the isolated pericyte progenitor cell of some embodiments of the invention, or the isolated population of cells of some embodiments of the invention in a chondrogenic culture medium such as a culture medium which comprises dexamethasone, ascorbic acid and TGFβ3, thereby generating the chondrocyte cells.

For example, for chondrogenic differentiation the pericyte progenitor cells (about 2×10⁵ cells) are centrifuged at 300 g for 5 minutes in 15 ml polypropylene falcon tubes to form a cell pellet. The cells are grown in the presence of 10 ng/ml TGFβ3 (Peprotech) in DMEM medium for 9 weeks with media changes twice a week without disturbing the cell mass. Alternatively, the pellets are cultured in medium containing 1% serum in addition to high-glucose Dulbecco's modified Eagle's medium supplemented with 10⁻⁷ M dexamethasone, 50 μg/ml ascorbate-2-phosphate, 40 μg/ml L-proline, 100 μg/ml sodium pyruvate, 50 mg/ml ITS+Premix (Collaborative Biomedical: 6.25 μg/ml insulin, 6.25 μg/ml transferrin, 6.25 ng/ml selenious acid, 1.25 mg/ml bovine serum albumin, and 5.35 mg/ml linoleic acid) and 10 ng/ml TGF-β3.

Additionally or alternatively, for chondrogenic differentiation sub-confluent pericyte progenitor cell cultures (from any passage, e.g., 1-9) are removed from the culture plates (without pre-collagenase treatment) as an intact layer, placed in suspension and fed with a medium containing alpha-MEM, supplemented with 15% fetal bovine serum (FBS), 50 μg/ml ascorbic acid, 10⁻⁷ M and dexamethasone.

According to some embodiments of the invention, the chondrocyte cells are characterized by positive von-Kossa staining.

According to an aspect of some embodiments of the invention, there is provided a method of generating myoblast cells. The method is effected by culturing the isolated pericyte progenitor cell of some embodiments of the invention, or the isolated population of cells of some embodiments of the invention in a culture medium which comprises horse serum, thereby generating the myoblast cells.

According to some embodiments of the invention, the myoblast cells are characterized by expression of markers such as MyoD and anti-smooth muscle myosin heavy chain.

For example, for myogenic differentiation, the pericyte progenitor cells of some embodiments of the invention (5×10⁴/cm²) are incubated in M-199 supplemented with 20% FBS for further growth and then for differentiation induction, the medium consisted of DMEM supplemented with 2% horse serum. Half of the medium was changed every 4 days for the following 7-10 days.

Additionally or alternatively, for myogenesis in vitro, the pericyte progenitor cells of some embodiments of the invention can be cultured in the presence of MS5 stromal cells (which are cultured in uncoated plates in a MEM medium supplemented by 10% FBS, 1% penicillin-streptomycin). Pericytes are co-cultured for 8-10 days in proliferation medium: 78.5% DMEM high-glucose, 10% FBS, 10% horse serum (GIBCO), 5% chicken embryo extract (CEE; Accurate), 1% penicillin-streptomycin, then 5-10 days in fusion medium: 96.5% DMEM high-glucose, 1% FBS, 1% HS, 0.5% CEE, 1% PS (Gibco). Half of the medium is replaced every 4 days, essentially as described in U.S. Patent Publication No. 2007/0264239, which is fully incorporated herein by reference.

For myogenesis in vivo, eight- to 12-week old SCID-NOD mice are used. The mice are anaesthetized by inhalation of isofluorane/O2. Cardiotoxin (1.5 μg/μl) is injected into the muscle one to 3 hours prior to cell transplantation. The pericyte progenitor cells of some embodiments of the invention are suspended in PBS (e.g., 35 μl) and then injected into the injured muscle. Mice are sacrificed 3 weeks after transplantation and muscle is harvested for immunohistochemistry analysis, essentially as described in U.S. Patent Publication No. 2007/0264239, which is fully incorporated herein by reference.

For myofiber regeneration in vivo, the pericyte progenitor cells of some embodiments of the invention (e.g., about 1×10⁴ cells) are injected into the gastrocnemius muscle of female NOD-SCID mice (6 to 8 weeks old) which had been injured by intramuscular injection of 15 μl of 50 μM cardiotoxin (Sigma) 2 hours earlier. Eighteen to twenty days after transplantation, the gastrocnemius muscles are harvested, flash frozen in liquid nitrogen-cooled 2-methylbutane, and serially sectioned. Spectrin staining can be used to detect human cell derived myofibers, essentially as described in U.S. Patent Publication No. 2007/0264239, which is fully incorporated herein by reference.

The ability of the pericyte progenitor cells of some embodiments of the invention to recover cardiac function, can be tested in an animal model essentially as described in U.S. Patent Publication No. 2007/0264239, which is fully incorporated herein by reference. Myocardial infarction is induced in anesthetized nude rats via ligation of the left anterior descending coronary artery. The pericyte progenitor cells of some embodiments of the invention in a PBS medium are immediately injected into the contracting wall bordering the infarct and into its center. One, 2, 6, and 12 weeks later one population of rats is sacrificed and hearts are harvested, frozen and serially cryosectioned. FISH (fluorescent in situ hybridization) of a human probe is used to track the human cells implanted in the myocardium. The same sections are used for detection of human spectrin or lamin. The sections are counterstained with DAPI to reveal all nuclei. The number of human spectrin+myofibers and the number of donor-derived nuclei are determined at different time points. Digitized images are evaluated to determine more effectively the area of engraftment within each injected heart. Anti-cardiac troponin I, anti-atrial natriuretic peptide (ANP), anti-Nkx2.5, anti-.alpha.-myosin heavy chain (a-MHC), anti-GATA-4, anti-connexin43 are used to investigate the acquisition of a myocardiac phenotype by the injected cells. Capillary density in the heart cryosections is monitored after anti-vWF, anti-CD144, anti-CD34 and anti-CD31 immunostaining, as is the expression of VEGF and VEGF receptor (KDR).

According to an aspect of some embodiments of the present invention there is provided a method of generating smooth muscle cells in vivo, comprising implanting the isolated pericyte progenitor cell of some embodiments of the invention, or the isolated population of cells of some embodiments of the invention in a subject in need thereof, on in a tissue of a subject in need thereof, thereby generating the smooth muscle cells in vivo.

According to some embodiments of the invention, the isolated pericyte progenitor cells are mixed with an extracellular matrix such as Matrigel, collagen and the like.

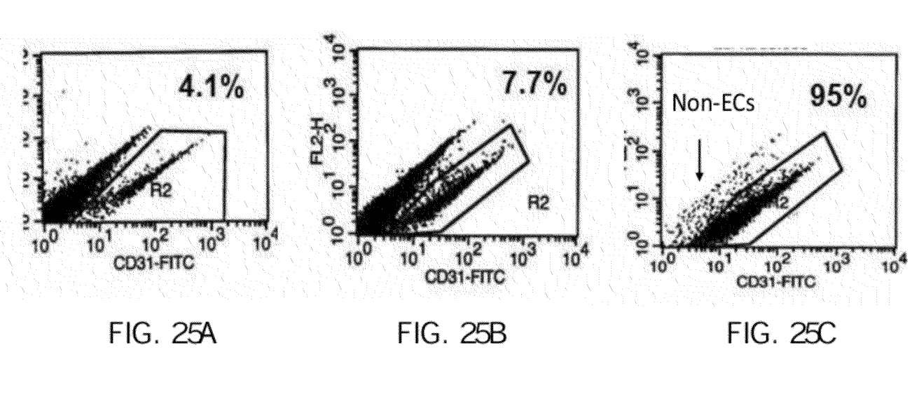

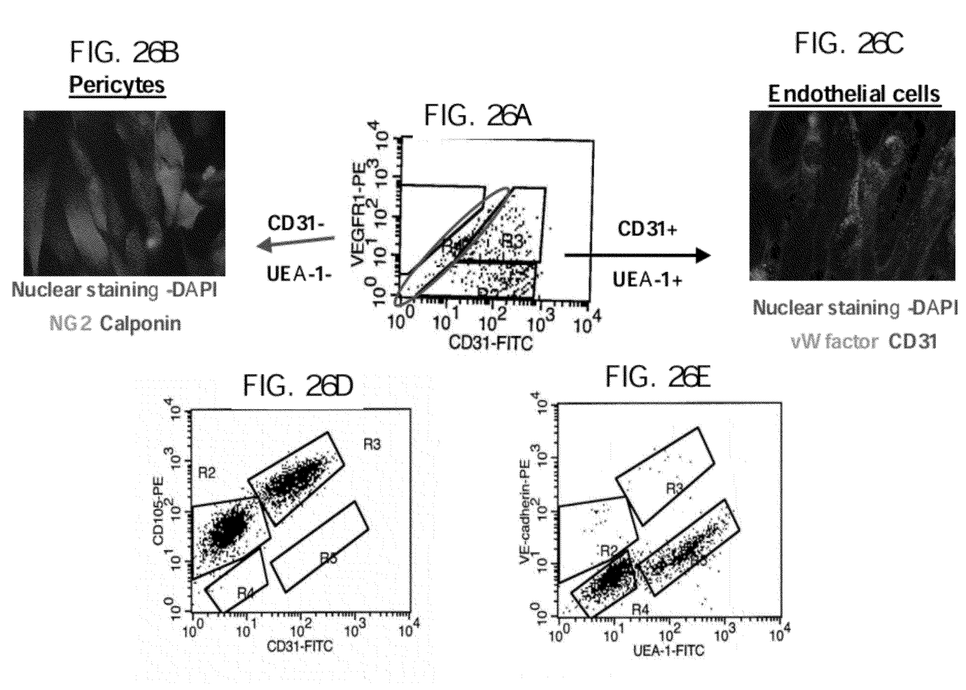

As shown in FIGS. 25A-C, 27A-E and described in Example 1, the present inventors have uncovered a novel method of isolating endothelial cells from embryoid bodies or pluripotent stem cells.

According to an aspect of some embodiments of the invention, there is provided a method of isolating an endothelial progenitor cell from embryoid bodies. The method is effected by: (a) isolating CD105+, CD73+ and/or CD105+/CD73+ cells from the embryoid bodies, to thereby obtain CD105+, CD73+ and/or CD105+/CD73+ cells, and; (b) isolating CD31+/UEA-1+/Ve-cadherin+ cells from the CD105+, CD73+ and/or CD105+/CD73+ cells, thereby isolating the endothelial progenitor cell from the pluripotent stem cells.

According to some embodiments of the invention, isolating endothelial progenitor cell is effected using pluripotent stem cells, which are induced to form embryoid bodies prior to isolation of the CD105+, CD73+ and/or CD105+/CD73+ cells.

As used herein the phrase “endothelial progenitor cell” refers to an endothelial cells having the ability to further differentiate to additional non-endothelial cells. Typically, endothelial cells are characterized by the expression of CD31, VE-Cadherin, UEA-1 and by lack of expression of αSMA.

According to some embodiments of the invention, for isolating endothelial cell progenitors, EBs from about day 7-26 are used.

Isolating the CD31+/Ulex europaeus lectin (UEA-1)+/VE-cadherin+ cells from the CD105+, CD73+ and/or CD105+/CD73+ cells can be performed using an antibody or antibodies which specifically bind CD31+, UEA-1+, and/or Ve-cadherin using any of the above described immuno-isolation methods.

CD31 [platelet/endothelial cell adhesion molecule, official symbol: PECAM1, also known as PECAM-1; FLJ58394] can be detected and/or isolated using specific antibodies such as CD31 antibody (ab32457; ABCAM), Clone MEM-05 (Abcam), Clone hc1/6 from AbD Serotec, or Clone JC70A (DAKO).

Ulex europaeus lectin (UEA-1) is a lectin. Non-limiting examples of antibodies which can specifically bind to cells expressing UEA-1 include NB110-13922 (Novus Biologicals), ab50683 (ABCAM), U4754 (Sigma-Aldrich) or FITC-L9006 (Sigma-Aldrich).

VE-cadherin [cadherin 5, type 2 (vascular endothelium), official symbol: CDH5, also known as 7B4; CD144; FLJ17376; CDH5, Swiss Prot: P33151] can be detected and/or isolated using specific antibodies such as clone EPR3111Y (Catalogue No. 2465-1, Epitomics), Anti-Human CD144 (VE-Cadherin) PE (Catalogue No. 12-1449, eBioscience), VE Cadherin antibody (ab33168, ABCAM) and PE-conjugated mouse anti-human VE-Cadherin (Catalogue No. FAB9381P, R&D, Systems).

According to some embodiments of the invention, the method further comprising culturing the CD105+, CD73+ and/or CD105+/CD73+ cells for one or two passages prior to the isolating the CD31+/UEA-1+/Ve-cadherin+ cells from the CD105+, CD73+ and/or CD105+/CD73+ cells.

According to some embodiments of the invention, the endothelial cells isolated by the method of some embodiments of the invention have an expression marker signature of CD31+/Ulex europaeus lectin (UEA-1)+/VE-cadherin+.

According to some embodiments of the invention, the endothelial cells isolated by the method of some embodiments of the invention express vW Factor [Von Willebrand factor, VWF, also known as VWD; F8VWF; is a glycoprotein which functions as both an antihemophilic factor carrier and a platelet-vessel wall mediator in the blood coagulation system]; Flk1 [VEGFR2, kinase insert domain receptor (a type III receptor tyrosine kinase), also known as CD309; VEGFR; KDR]; CD34; and/or Flt-1 [VEGFR1, fms-related tyrosine kinase 1 (vascular endothelial growth factor/vascular permeability factor receptor), also known as FLT].

According to some embodiments of the invention, the endothelial progenitor cells isolated by the method of some embodiments of the invention have an expression marker signature of CD31+/Ulex europaeus lectin (UEA-1)+/VE-cadherin+/vW Factor/Flk1+/CD34+/Flt-1+.

The endothelial progenitor cells can be cultured under non-differentiating conditions such as on a matrix (such as fibronectin extracellular matrix or gelatin) in the presence of a culture medium such as endothelial culture medium such as M-199 (Biological Industries, Israel) supplemented with FBS and ECGS and heparin; EBM-2 (Lonza, MD, USA) with supplements; or M-199 supplemented with FBS and ECGS, heparin and TGFβ3 inhibitor SB431542 (Tocris Biosciences, Bristol UK). The endothelial cells can be passaged every about 5-10 days as described in the Examples section which follows and in Daylon et al., 2009 (Nature Biotechnology, advanced online publication, which is fully incorporated herein by reference).

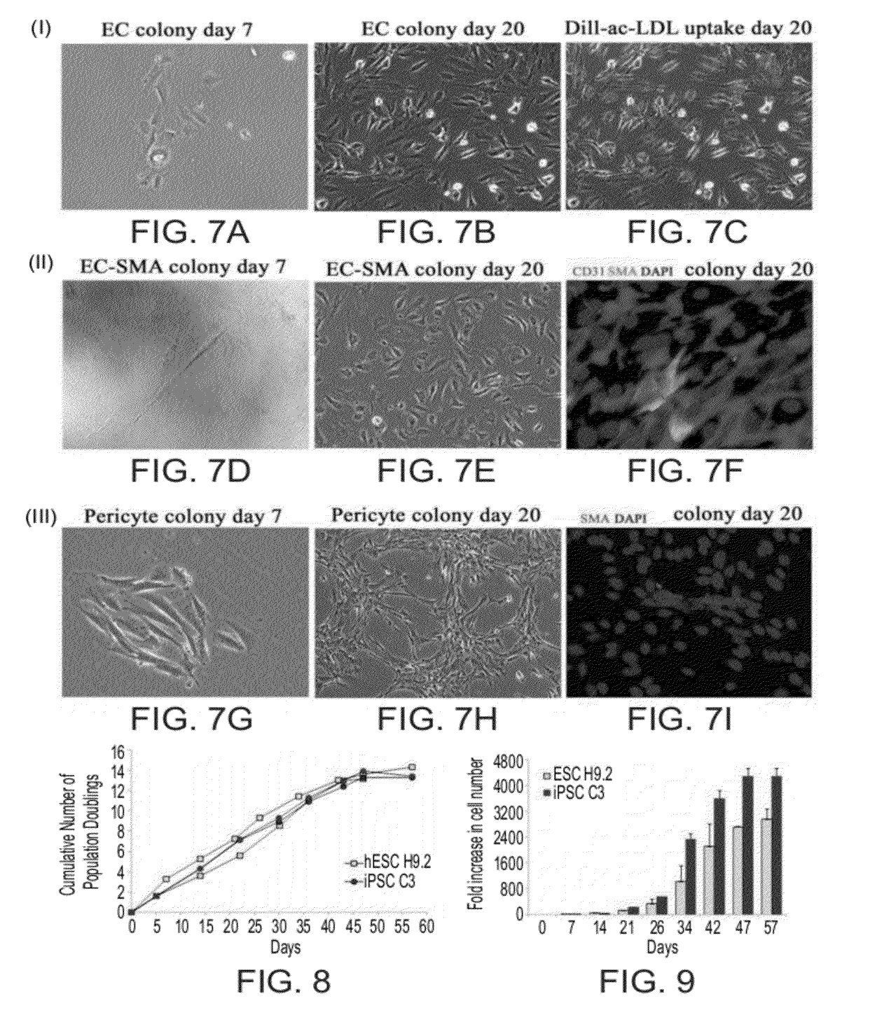

According to some embodiments of the invention, while in culture the endothelial progenitor cells can differentiatiate into cells which express αSMA (See for example, FIG. 7F, Example 2 of the Examples section which follows).

According to an aspect of some embodiments of the invention, there is provided an isolated population of endothelial progenitor cells generated according to the method of some embodiments of the invention.

According to some embodiments of the invention, the isolated population of endothelial progenitor cells comprises at least 80%, 81%, 82%, 83%, 84%, 85%, 86%, 87%, 88%, 89%, 90%, 91%, 92%, 93%, 94%, 95%, 96%, 97%, 98%, 99%, e.g., 100% of endothelial progenitor cells having the CD31+/Ulex europaeus lectin (UEA-1)+/VE-cadherin+ signature.

According to some embodiments of the invention, the isolated population of endothelial progenitor cells comprises at least 80%, 81%, 82%, 83%, 84%, 85%, 86%, 87%, 88%, 89%, 90%, 91%, 92%, 93%, 94%, 95%, 96%, 97%, 98%, 99%, e.g., 100% of endothelial progenitor cells having the CD31+/Ulex europaeus lectin (UEA-1)+/VE-cadherin+/vW Factor+/Flk1+/CD34+/Flt-1+ signature.

According to some embodiments of the invention, the isolated population of endothelial progenitor cells comprises at least 80%, 81%, 82%, 83%, 84%, 85%, 86%, 87%, 88%, 89%, 90%, 91%, 92%, 93%, 94%, 95%, 96%, 97%, 98%, 99%, e.g., 100% of endothelial progenitor cells having the CD31+/Ulex europaeus lectin (UEA-1)+/VE-cadherin+/vW Factor+/Flk1+/CD34+/Flt-1+/CD105+/CD73+/NG2−/PDGFR-13−/CD90− signature.

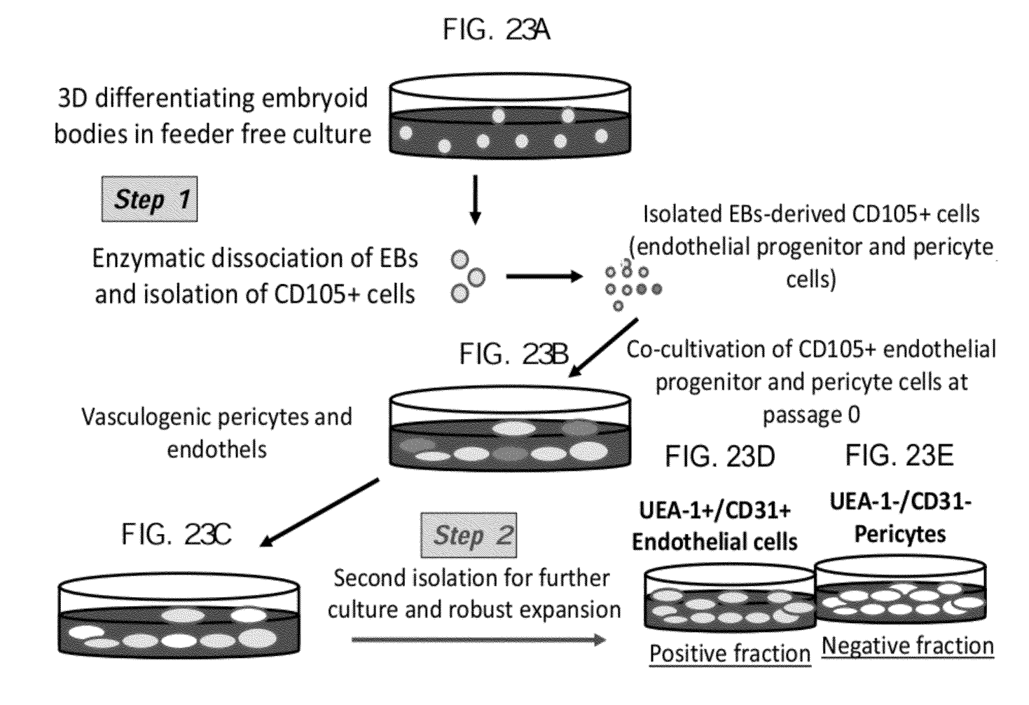

As shown in FIGS. 23A-E, 24A-C, 26A-E, and described in Example 6 of the Examples section which follows, the present inventors have identified a novel method of co-derivation of pericyte and endothelial progenitor cells from embryoid bodies. Such derivation of pericyte and endothelial progenitor cells from a pluripotent cell source was never described. The pericyte and endothelial cells can be further expanded and used for various applications either as separated population of cells or as a combined population of cells in various ratios therebetween, such as 1:2, 1:1, 2:1, 1:3, 3:1 or pericyte/endothelial progenitor cells.

According to an aspect of some embodiments of the invention, there is provided a method of co-derivation of pericyte and endothelial progenitor cells. The method is effected by: (a) isolating CD105+, CD73+ and/or CD105+/CD73+ cells from embryoid bodies, to thereby obtain CD105+, CD73+ and/or CD105+/CD73+ cells; (b) isolating a CD31+/UEA-1+/Ve-cadherin+ cells from the CD105+, CD73+ and/or CD105+/CD73+ cells, to thereby isolate the endothelial progenitor cells; (c) isolating CD31− cells from the CD105+, CD73+ and/or CD105+/CD73+ cells, to thereby isolate the pericyte progenitor cells; thereby co-derivation of the pericyte and endothelial progenitor cells.

According to some embodiments of the invention, the method uses pluripotent stem cells, from which the embryoid bodies are generated prior to isolating CD105⁺, CD73+ and/or CD105+/CD73+ cells from the embryoid bodies.

According to some embodiments of the invention, for co-derivation of pericyte and endothelial progenitor cells the CD105+, CD73+ and/or CD105+/CD73+ cells are isolated from EBs of day about 7-26 of EBs differentiation.

According to some embodiments of the invention, steps (b) and (c) are performed on two separated culture dishes or flasks, in order to isolate the two different populations of pericyte progenitors and endothelial progenitors.

According to some embodiments of the invention, the method further comprising: culturing the CD31+/UEA-1+/Ve-cadherin+ cells, to thereby expand the endothelial progenitor cells.

According to some embodiments of the invention, isolating and/or culturing the CD31+/UEA-1+/Ve-cadherin+ cells results in an isolated population of endothelial progenitor cells comprises at least 80%, 81%, 82%, 83%, 84%, 85%, 86%, 87%, 88%, 89%, 90%, 91%, 92%, 93%, 94%, 95%, 96%, 97%, 98%, 99%, e.g., 100% of endothelial progenitor cells as described above (e.g., having the CD31+/Ulex europaeus lectin (UEA-1)+/VE-cadherin+ signature).

According to some embodiments of the invention, the method further comprising culturing the CD31− cells, to thereby expand the pericyte progenitor cells.

It should be noted that the pericyte progenitor cells isolated by co-derivation can be cultured on low-adhesive culture plates/flasks which can be non-coated.

According to some embodiments of the invention, isolating and/or culturing the CD31− cells results in an isolated population of pericyte progenitor cells comprises at least 80%, 81%, 82%, 83%, 84%, 85%, 86%, 87%, 88%, 89%, 90%, 91%, 92%, 93%, 94%, 95%, 96%, 97%, 98%, 99%, e.g., 100% of pericyte progenitor cells as described above (e.g., having the CD105+/CD73+/CD31−/αSMA− signature).

Thus, the teachings of the invention can be used to generate two populations of cells, wherein one population comprises pericyte progenitor cells and the other population comprises endothelial progenitor cells.

According to an aspect of some embodiments of the invention, there is provided an isolated population of pericyte progenitor cells generated by co-derivation as described above.

According to an aspect of some embodiments of the invention, there is provided an isolated population of endothelial progenitor cells generated by co-derivation as described above.

According to an aspect of some embodiments of the invention, there is provided an isolated population of pericyte and endothelial progenitor cells generated according to the method of some embodiments of the invention.

According to an aspect of some embodiments of the invention, there is provided a cell culture comprising a culture medium and the isolated endothelial progenitor cell of some embodiments of the invention, or the isolated population of cells of some embodiments of the invention.

The medium can be any liquid medium suitable for culturing the endothelial progenitor cells. Non-limiting examples of such a medium include, but are not limited to the endothelial cell medium M-199 medium supplemented with FBS and ECGS and heparin.

According to an aspect of some embodiments of the invention, there is provided a cell culture comprising a culture medium and the isolated endothelial and pericyte progenitor cell of some embodiments of the invention, or the isolated population of cells of some embodiments of the invention.

The medium can be any liquid medium suitable for culturing the endothelial and pericyte progenitor cells. Non-limiting examples of such a medium include, but are not limited to the endothelial cell medium M-199 medium supplemented with FBS and ECGS and heparin.

As shown in FIGS. 17A-E, 18A-E, 33A-D, 34A-B, 36A-C and described in Example 4 of the Examples section which follows, the pericyte and endothelial cells generated according to the present teachings were shown capable of forming vascular tubes and constructs both in vitro and in vivo.

The isolated pericyte and/or endothelial progenitor cells of some embodiments of the invention can be used for repairing and/or regenerating vascular tissues.

According to an aspect of some embodiments of the invention, there is provided a method of treating a pathology requiring vascular tissue regeneration and/or repair. The method is effected by administering to a subject having the pathology the isolated pericyte progenitor cell of some embodiments of the invention, the isolated population of pericyte progenitor cells of some embodiments of the invention, the cell culture of some embodiments of the invention, the isolated population of pericyte and endothelial progenitor cells of some embodiments of the invention, the isolated population of endothelial progenitor cells of some embodiments of the invention, or a pharmaceutical composition comprising same, thereby treating a pathology requiring vascular tissue regeneration and/or repair.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology.

Non-limiting examples of diseases/pathologies/conditions which require vascular tissue regeneration and/or repair include ischemia, diabetes, diabetic microangiopathy, peripheral arterial disease, cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med. Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9), vascular injury, vascular repair due to removal of cancerous tissue and the surrounding vasculature, tissue fibrosis, cancer, and Alzheimer's disease, chronic lung disease, trauma, injury, cancer, diabetes, blood coagulation related-disorders (e.g., over coagulation), and myofiber regeneration.

Administration of the cells of some embodiments of the invention can be effected using any suitable route such as intravenous, intra cardiac, intra peritoneal, intra kidney, intra gastrointestinal track, subcutaneous, transcutaneous, intramuscular, intracutaneous, intrathecal, epidural and rectal.

The cells of some embodiments of the invention can be derived from either autologous sources such as self skin cells which are induced to become iPSCs and which are further used by the method of some embodiments of the invention or from allogeneic sources such as bone marrow or other cells derived from non-autologous sources. Since non-autologous cells are likely to induce an immune reaction when administered to the body several approaches have been developed to reduce the likelihood of rejection of non-autologous cells. These include either suppressing the recipient immune system or encapsulating the non-autologous cells or tissues in immunoisolating, semipermeable membranes before transplantation.

Encapsulation techniques are generally classified as microencapsulation, involving small spherical vehicles and macroencapsulation, involving larger flat-sheet and hollow-fiber membranes (Uludag, H. et al. Technology of mammalian cell encapsulation. Adv Drug Deliv Rev. 2000; 42: 29-64).

Methods of preparing microcapsules are known in the arts and include for example those disclosed by Lu M Z, et al., Cell encapsulation with alginate and alpha-phenoxycinnamylidene-acetylated poly(allylamine). Biotechnol Bioeng. 2000, 70: 479-83, Chang T M and Prakash S. Procedures for microencapsulation of enzymes, cells and genetically engineered microorganisms. Mol. Biotechnol. 2001, 17: 249-60, and Lu M Z, et al., A novel cell encapsulation method using photosensitive poly(allylamine alpha-cyanocinnamylideneacetate). J Microencapsul. 2000, 17: 245-51.

For example, microcapsules are prepared by complexing modified collagen with a ter-polymer shell of 2-hydroxyethyl methylacrylate (HEMA), methacrylic acid (MAA) and methyl methacrylate (MMA), resulting in a capsule thickness of 2-5 μm. Such microcapsules can be further encapsulated with additional 2-5 μm ter-polymer shells in order to impart a negatively charged smooth surface and to minimize plasma protein absorption (Chia, S. M. et al. Multi-layered microcapsules for cell encapsulation Biomaterials. 2002 23: 849-56).

Other microcapsules are based on alginate, a marine polysaccharide (Sambanis, A. Encapsulated islets in diabetes treatment. Diabetes Thechnol. Ther. 2003, 5: 665-8) or its derivatives. For example, microcapsules can be prepared by the polyelectrolyte complexation between the polyanions sodium alginate and sodium cellulose sulphate with the polycation poly(methylene-co-guanidine) hydrochloride in the presence of calcium chloride.

It will be appreciated that cell encapsulation is improved when smaller capsules are used. Thus, the quality control, mechanical stability, diffusion properties, and in vitro activities of encapsulated cells improved when the capsule size was reduced from 1 mm to 400 μm (Canaple L. et al., Improving cell encapsulation through size control. J Biomater Sci Polym Ed. 2002; 13: 783-96). Moreover, nanoporous biocapsules with well-controlled pore size as small as 7 nm, tailored surface chemistries and precise microarchitectures were found to successfully immunoisolate microenvironments for cells (Williams D. Small is beautiful: microparticle and nanoparticle technology in medical devices. Med Device Technol. 1999, 10: 6-9; Desai, T. A. Microfabrication technology for pancreatic cell encapsulation. Expert Opin Biol Ther. 2002, 2: 633-46).

The isolated pericyte progenitor cell of some embodiments of the invention, the isolated pericyte progenitor cell of some embodiments of the invention, the isolated population of pericyte progenitor cells of some embodiments of the invention, the cell culture of some embodiments of the invention, the isolated population of pericyte and endothelial progenitor cells of some embodiments of the invention, and/or the isolated population of endothelial progenitor cells of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the isolated pericyte progenitor cell of some embodiments of the invention, the isolated population of pericyte progenitor cells of some embodiments of the invention, the cell culture of some embodiments of the invention, the isolated population of pericyte and endothelial progenitor cells of some embodiments of the invention, and/or the isolated population of endothelial progenitor cells of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, vascular tissue, brain tissue, retina, skin tissue, hepatic tissue, pancreatic tissue, bone, cartilage, connective tissue, blood tissue, muscle tissue, cardiac tissue brain tissue, renal tissue, pulmonary tissue, gonadal tissue, hematopoietic tissue.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (the isolated population of pericyte progenitor cells of some embodiments of the invention, the cell culture of some embodiments of the invention, the isolated population of pericyte and endothelial progenitor cells of some embodiments of the invention, the isolated population of endothelial progenitor cells of some embodiments of the invention) effective to prevent, alleviate or ameliorate symptoms of a disorder (e.g., a pathology requiring vascular tissue regeneration and/or repair) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Thus, the teachings of the invention are of both clinical, basic research and industrial importance. The methods of the invention offer scalable culture systems for clinical and industrial purposes.

The isolated pericyte progenitor cells of some embodiments of the invention and/or cells differentiated therefrom can be used to stabilize blood vessels [see e.g., Darland, et al., Angiogenesis (2001) 4, 11-20; Carmeliet, Nat Med (2003) 9, 653-660; Jain, Nat Med (2003) 9, 685-693; Sieminski, et al., Tissue Eng (2002) 8, 1057-1069; Koike, et al., Nature (2004) 428, 138-139; Black, et al., FASEB J (1998) 12, 1331-1340; Shinoka, et al., Artif Organs (2002) 26, 402-406)]; In addition, the isolated endothelial cells of some embodiments of the invention can induce the differentiation of undifferentiated mesenchymal cells into smooth muscle cells [See e.g., Flamme, et al., J Cell Physiol (1997) 173, 206-210; Rossant, et al., Curr Opin Genet Dev (2003) 13, 408-412; Hirschi, et al., J Cell Biol (1998) 141, 805-814; Darland, et al., Dev Biol (2003) 264, 275-288)]. Moreover, the isolated pericyte progenitor cells can induce differentiation into smooth muscle cells in vivo as shown in FIGS. 33C-D, 34A-B and described in Example 5 of the Examples section which follows.

Drug Screening

The pericyte and/or endothelial progenitor cells of some embodiments of the invention can be used to screen for drugs having an effect on tube formation and/or regeneration of vascular tissue.

In vitro models: Normal pluripotent stem cells (hESCs or iPSCs) (e.g., healthy, derived form a healthy individual devoid of any known disease or pathology) or disease-related pluripotent stem cells (e.g., cells derived from a subject having a known disease, such as a genetic disease, a vascular disease, cancer, diabetes, arthrosclerosis, metabolic disease) can be used to generate pericyte progenitor cells, and/or endothelial progenitor cells which are further used to generate vascular tubes in vitro, e.g., using a matrix such as Matrigel (BD Biosciences). Briefly, cultured PSC-derived pericytes or EPCs are seeded on Matrigel coated slides or 24 well dishes in M-199 medium supplemented with 20% FBS without ECGS and heparin. Tube formation is documented 2, 12 and 48 hours post seeding. Various drugs can be added before seeding, at pre-determined time points after seeding (i.e., during tube formation), and/or after the tubes are formed. The effect of the drugs on the kinetic of tube formation, and/or on tube structure can be detected by monitoring the structure/morphology of the formed microtubes. Drug molecules capable of repairing tube formation, and/or kinetic of tube formation can be selected.

In vivo models: Pericyte progenitor cells, and/or endothelial progenitor cells which are generated from normal or diseased pluripotent stem cells can be mixed with a matrix (e.g., Matrigel assay) and further injected to the back or lateral flank of NOD/SCID mice (e.g., 6-8 old). Drugs can be injected to the implanted animals at various time points before, during or following implantation, and the effect of the drug on tube formation can be evaluation by various histological and/or immunohistochemical assays.

Functional assays—Pericyte progenitor cells, and/or endothelial progenitor cells which are generated from pluripotent stem cells can be used to generate vasculature ex vivo and/or in vivo. These cells can be used to establish clinical transplantation protocols for treatment of vascular disorders.

Ex vivo vascular formation—Briefly, scaffolds or tissue grafts (e.g., biological, synthetic, biodegradable and the like) can be seeded with the pericyte progenitor cells, and/or endothelial progenitor cells and tube formation can be monitored using known histochemical/immunological methods.

In vivo vascular formation—Briefly, scaffolds or tissue grafts (e.g., biological, synthetic, biodegradable and the like) can be seeded with the pericyte progenitor cells, and/or endothelial progenitor cells and further implanted in an animal model/subject in need thereof to test the ability of the vascular cells to repair, regenerate and/or form a vascular tissue. It should be noted the cells can be cultured ex vivo prior to being implanted in a subject. Additionally or alternatively, the scaffold or tissue graft can be implanted to the subject/animal model without the cells and the cells can then be administered to the site of implantation or in vicinity thereto after graft implantation.

Tissue grafts, tissue repair and/or regeneration—The following are non-limiting examples of tissue grafts which can be seeded with the pericyte progenitor cells, and/or endothelial progenitor cells prior to, concomitant with, or following implantation of the tissue grafts: cardiac muscle tissue graft (for generation of vasculature for cardiac muscle), pancreatic tissue graft (islets of Langerhans), liver graft, bone graft, cartilage graft, skeletal muscle graft, and lung graft.

Use of the pericyte progenitor cells, and/or endothelial progenitor cells of some embodiments of the invention for understanding the pathogenesis of various diseases—Normal and diseased pluripotent stem cells (as described above) can be used to generate pericyte progenitor cells, and/or endothelial progenitor cells which are used in various models to understand the pathogenesis of a disease. For example, tube formation can be compared between pericyte progenitor cells, and/or endothelial progenitor cells which are generated from iPSCs derived from a diabetic subject and from iPSCs of a healthy individual.

The pericyte progenitor cells, and/or endothelial progenitor cells of some embodiments of the invention can be used for better understanding developmental processes of vasculogenesis as well as identification of interactions between the blood vessel cellular compartments.

Additional therapeutic uses of the pericyte progenitor cells, and/or endothelial progenitor cells of some embodiments of the invention: tissue reconstruction, reconstructive surgery,

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure. As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Human pluripotent stem cell (PSCs) culture and differentiation—Human ESC H9.2 (passages 29+36-60, i.e., the H9 cell line was cloned on passage 29, and the H9.2 cell line clone was used at passages 36-60), 16 (passages 50-71) or human foreskin fibroblasts derived iPSC (Germanguz et al, J Cell Mol. Med. 2009 Dec. 11. [Epub ahead of print]), clones C2 and C3 (passages 17-38) and hair follicle keratinocyte derived iPSC clone KTR13 (passages 30-49) were grown on mitomycin C (1 mg/ml) inactivated mouse embryonic fibroblasts (MEF) in ESC culture medium consisting of: advanced DMEM/F12 (Biological Industries, Biet HaEmek, Israel) supplemented with 20% knockout serum replacement (GIBCO™ Knockout™ Serum Replacement), 1× non-essential amino acids (Gibco), 1×1-glutamine (Invitrogen), 1×β-mercaptoethanol (Gibco) and 4 ng/ml FGF-2 (R&D systems) without antibiotics.

Embryoid bodies formation and differentiation—To induce spontaneous embryoid bodies (EBs) formation and differentiation, PSCs were removed from MEF feeder by 0.2% Collagenase IV (Gibco Invitrogen Corporation, Grand Island N.Y., USA) and were suspended in low attachment culture dishes in differentiating medium consisting of DMEM, 20% FBS, 1× non-essential amino acids, 1×1-glutamine, 1×β-mercaptoethanol without antibiotics. Culture medium was changed every 3 days.

EBs dissociation protocols—To optimize the dissociation step for obtaining single cell suspensions, three methods of dissociating EBs were tested. These include incubation of EBs for 20 minutes at 37° C. on shaker with either (1) 0.5% Trypsin/EDTA (Sigma), (2) non-enzymatic solution (Sigma, catalogue No. C5914) or (3) 1 mg/ml collagenase B and 150 U/ml DNAse I (Catalogue No. 2139, Roche), followed by addition of Trypsin-EDTA (0.05%) for another 5 minutes. To minimize cell aggregation the dissociated EBs were then passed several times through a 20-Gauge needle and filtered through PBS/0.5% FBS pre-washed 0.45 cell-strainers (BD Biosciences). Cell viability was determined by Trypan Blue assay, and the percentages of live CD31⁺ endothelial progenitor cells (EPC) or CD105+ cells were analyzed by flow cytometry upon dissociation with Colagenase B.

Pericyte and endothelial progenitor cell isolation (for co-derivation)—At indicated time points of spontaneous differentiation in 3D cultures (between days 7-26), single-cell suspensions were made from differentiated hPSC (EBs) by treatment with 1 mg/ml collagenase B and 150 U/ml DNAse I as detailed above. CD105+ cells were isolated from differentiated hPSC (EBs) by using MACS MicroBeads and MACS columns (Miltenyi Biotec, anti-human CD105 MicroBeads, Catalogue No. 130-051-201, anti-human CD31 MicroBeads Catalogue No. 130-091-935, MS columns 130-042-201 and LS columns 130-042-401), according to the manufacturer's instructions. As determined by flow cytometry, the purity of isolated CD105⁺ cells was generally 60-70% at a single column, and >96% after the second column of CD105-based isolation.

For further expansion of each population, a second isolation step was preformed, CD31+CD105+ endothelial cells were sorted/isolated from mixed culture of CD105+ isolated cells at passage 1-2 by magnetic separation using anti-CD31 MicroBeads conjugated antibodies or by sorting using FITC or PE-conjugated anti-human CD31 antibodies (purity of CD31⁺>97%). Similarly, UEA-1 based isolation was performed by cell sorting using UEA-1-FITC which resulted in CD105+/UEA-1+ endothelial cells and CD105+/UEA-1− pericyte progenitor cells.

Isolated CD31+CD105+ endothelial cells were further expanded. PSC-derived CD31+/CD105+EPCs from second isolation were cultured in coated fibronectin or gelatin plates in a medium such as EBM-2 (Catalogue No. CC-3156, Lonza, Walkerville, Md., USA) or EC M-199 growth media both supplemented with 10 μM SB431542 (Catalogue No. 1614, Tocris Biosciences, Bristol, UK) for expansion of endothelial cells as described (Daylon J., et al., 2010, Nat Biotechnol 28: 161-166).

CD105+CD31− pericytes from second isolation step (which used CD31 antibodies) were further expanded in EC M-199 growth media in uncoated plates.

Culturing of isolated CD105-F/CD31− and CD105-F/CD31÷ cells obtained by sorting of CD105÷ cells—CD105+ isolated/sorted mixed culture of EPCs and pericytes; CD31+/CD105+ isolated/sorted endothelial progenitor cells (EPCs); or CD105+/CD31− isolated/sorted pericyte progenitor cells were seeded on human fibronectin (Millipore, Billerica, Mass.) coated culture dishes and cultured in endothelial cell (EC) M-199 media (Catalogue No. 01-085-1A, Biological Industries, Bet-Haemek, Israel) containing 20% defined-fetal bovine serum (FBS) (HyClone, Utah, USA), 1% Pen-Strep, 1% 1-glutamine, 1 mM HEPES (Biological Industries) 20 U/ml heparin (Sigma-Aldrich) and 50 μg/ml endothelial cell growth supplement (ECGS) (Catalogue No. BT-203, Biomedical Technologies Inc., Stoughton, Mass.).

Isolated cells of CD105-F/CD31− obtained by expansion of CD105÷ cells in culture—For direct isolation of pericyte progenitor cells the mixed population CD105+/CD31+ and CD105+/CD31− cells were co-cultured in EC M-199 growth media. As analyzed by flow cytometry, CD105+CD31− dominated the culture within 1-2 passages, in accordance with the initial percentage of isolated CD105+CD31− within dissociated EBs.

Immunocytochemistry and Immunohistochemistry—For fluorescence microscopy, 17 days old iPSC (KTR13) EBs, PSC-derived pericytes or cultured endothelial cells (ECs) on glass coverslips were fixed with 4% paraformaldehyde, washed 3 times with PBS and permeabilized with PBS 0.1% Triton X-100 for 10 minutes (Sigma). The following uncoupled anti-human primary antibodies were then used: rabbit-anti human NG2 (millipore, Catalogue No. AB5320, 1:50), rabbit anti-human PDGFR-β (clone Y92, Catalogue No. 1469-1), calponin (clone EP798Y, Catalogue No. 1806-1), CD90 (clone EPR3133, Catalogue No. 2695-1) and MHC class I (clone EPR1394Y, Catalogue No. 2307-1) all were used at a 1:100 dilution and obtained from Epitomics, Burlingame, Calif., USA; anti-Tie-2 (1:100, R&D Systems) mouse anti-human α-SMA (DAKO, clone, 1A4, Catalogue No. M0851,), smooth muscle myosin heavy chain or CD31 (Dako, 1:50), and mouse anti-human CXCR4 (clone 12G5, R&D Systems, 1:300). FITC-conjugated Ulex europaeus lectin (UEA-1-FITC) was also used as an endothelial cell marker (Sigma, 1:200). The secondary conjugated antibodies included: Alexa-488 conjugated donkey anti-mouse, Alexa-488 conjugated donkey anti-rabbit, Alexa-488 conjugated donkey anti-goat (1:100, Invitrogen), Alexa-488 conjugated goat anti-rabbit, and Cy-3 conjugated donkey anti-mouse (1:100, Jackson). Labeled Dil-Ac-LDL (5 μg/ml) was used for identification of endothelial cells according to the manufacturer instructions (Biomedical Technologies Inc). CM-Dil labeling was performed according to the manufacturer instructions (Molecular probes). Slides were mounted in mounting medium (Catalogue No., 53023, Dako) and observed on an epifluorescence microscope (Ziess). Adherent cultures were viewed by Axiovert 200 equipped with AxioCam MRm camera.

For evaluations of implanted cells, at the indicated time points Matrigel implants were harvested and fixed in 4% formalin (BioLab LTD). Sections were labeled with primary antibodies O.N. at 4° C. Primary antibodies included the following: mouse anti-human CD31 (1:50, did not cross react with murine CD31, n=38 tests), rabbit anti-human vW factor (1:300), mouse anti-human α-SMA (1:100) and mouse anti-human CD34 (1:50) were purchased from Dako. Rabbit anti-human MHC class I (1:100, Epitomics). Nuclei labeling with DAPI (40,6-diamino-2-phenylindole dihydrochloride, Molecular Probes, 1:1000) for 5 minutes at room temperature (RT), and an isotype-matched negative control or irrelevant isotype-matched antibody (e.g. mouse anti-human CD45, Dako) was performed with each immunostaining.

Flow cytometry—At the indicated time points, differentiated EBs were dissociated with Collagenase B and DNAse I as described above, washed in PBS and labeled with various combinations of coupled antibodies. Antibodies were used according to the manufacturer instructions and included the following: Anti-CD31-FITC (Catalogue No. 555445, BD Pharmingen), anti-CD73-PE (Catalogue No. 550257, BD Pharmingen), anti-CD90-PE (Catalogue No. 12-0909-73, eBioscience), anti-CD45-PE (Catalogue No. 555483, BD Pharmingen), anti-CD105-APC (Catalogue No. 17-1057-42, eBioscience), anti-CD146-PE (Catalogue No. 550315, BD Pharmingen), anti-CD44-PE (Catalogue No. 12-0441-81, eBioscience), anti-CD29-PE (Catalogue No. 12-0297-7, eBioscience), anti-CD56-FITC (Catalogue No. 11-0569-73, eBioscience), anti-CD14-FITC (Catalogue No. 11-0149-41, eBioscience), anti-CD34-FITC (Catalogue No. 555821, BD Pharmingen), VE-Cadherin (Catalogue No. FAB9381P, R&D, Systems) isotype control antibodies included PE-conjugated mouse IgG2B (Catalogue No. IC0041P, R&D, Systems), FITC-conjugated mouse IgG11c (Catalogue No. BD Pharmingen). Analyses were carried out using Facscalibur flow cytometer and CELLQuest program (Becton Dickinson).

Matrigel assays in vitro and in vivo—Cultured PSC-derived pericytes or EPCs were seeded on Matrigel coated slides or 24 well dishes in M-199 supplemented with 20% FBS without ECGS and heparin. Tube formation was documented 2, 12 and 48 hours post seeding. Slides were fixed in 4% paraformaldehyde and labeled with mouse anti-human CD31 (1:100, DAKO). Pericytes were pre-labeled with CFSE according to the manufacturer instructions.

For in vivo Matrigel assay, endothelial cells, either human umbilical vein endothelial cells (HUVEC) or PSC-derived EC and/or PSC-derived pericytes were removed from culture dishes by 0.05% trypsin, washed and re-suspended in 250 μl phenol-red free Matrigel (BD Biosciences), either alone (3−5×10⁵ EC, 6−8×10⁵ pericytes) or mixed. Matrigel mixture was then injected to the back or lateral flank of 6-8 old NOD/SCID mice. Implants were removed on the indicted days and fixed in 4% formalin, embedded in paraffin, sectioned and stained with hematoxylin and Eosin or further immunolabeled. Empty Matrigel served as control implants.

Adipogenic differentiation and Oil Red 0 staining—Pericytes were seeded (2×10⁵ cells/cm²) on culture dish, in the presence of 0.5 mM IBMX, 10 μg/ml Insulin, 10⁻⁶ M Dexamethasone, and 0.1 mM Indomethacin in DMEM/F12 medium (Biological Industries, Biet HaEmek, Israel) containing 10% FBS for 4 weeks, with media changes twice a week. Adipogenic differentiation was assessed by accumulation of lipid-rich vacuoles within the cells after Oil Red 0 staining as follows: cells were rinsed once with PBS, fixed with 4% Paraformaldehyde (PFA) for 20 minutes, rinsed again and stained with Oil Red 0 solution for 10 min in room temperature. Staining solution was removed and the cells were washed 5 times with water.

Osteogenic differentiation in vitro—Pericytes were seeded at low density (2×10⁴-3×10⁴ cells/cm²) on culture dish, in the presence of 10 mM β-glycerol-phosphate and 0.1 μM Dexamethasone (Catalogue No. D4902, Sigma-Aldrich) in GMEM BHK-21 medium (Catalogue No. 21710, Gibco) containing 10% FBS for 4 weeks, with media changes twice a week. Cell cultures were assayed for mineral content by Alizarin red staining as follows: treated cells were rinsed once with PBS, fixed with 4% PFA for 20 minutes, rinsed again and stained with 2% Alizarin red solution for 15 minutes in room temperature. Staining solution was removed and the cells were washed several times with water.

For osteogenesis in vivo pericytes were cultured in osteogenic differentiation medium (10 mM β-glycerol-phosphate and 0.1 μM Dexamethasone (Catalogue No. D4902, Sigma-Aldrich) in GMEM BHK-21 medium (Catalogue No. 21710, Gibco) for 3 or 14 days. Treated cells were then removed from culture dishes and mixed with 250 μl Matrigel ((BD Biosciences) at a concentration of 3×10⁵-10⁶ cells/Matrigel implant of 250 μl, which was injected subcutaneously into immunodeficient 10-12 weeks old NOD/SCID mice. Implants were harvested after 1-2 weeks, fixed in 4% formaldehyde, embedded in paraffin, sectioned and stained with H&E or 2% Alizarin red solution for 15 min in room temperature.

Chondrogenic differentiation, hematoxylin and eosin and Alcian blue staining—For chondrogenic differentiation, 2×10⁵ pericytes were centrifuged at 300 g for 5 minutes in 15 ml polypropylene falcon tubes to form a cell pellet. The cells were grown in the presence of 10 ng/ml TGFβ3 (Peprotech) in DMEM medium (Gibco) for 9 weeks with media changes twice a week without disturbing the cell mass. Cell sections were made after fixing the cell pellets with 4% PFA and embedding the cell mass in low melting point agarose (1.5%). Hematoxylin and Eosin and Alcian blue stainings were then performed.

Myogenic differentiation—PSC-derived pericytes (5×10⁴/cm²) were incubated in M-199 supplemented with 20% FBS for further growth. Differentiation medium consisted of DMEM supplemented with 2% horse serum. Half of the medium was changed every 4 days for the following 7-10 days. Fixed cells (4% paraformaldehyde) were labeled with anti-smooth muscle myosin heavy chain (clone, SMMS-1 Catalogue No. M3558, Dako) and anti-MyoD (clone 5.8A, Catalogue No. sc-32758, Santa-Cruz) for evaluation of myotubes formation.

RT-PCR—Total RNAs were isolated using Trizol/Tri-reagent (Invitrogen) and reverse transcribed reverse transcribed by the iScript™ cDNA synthesis kit (BIO-RAD). RT-PCR was performed by DreamTaq™ Green Master Mix (Fermentas, Ontario, Canada). The sequences of oligonucleotide primers used for PCR are listed in Table 1 below.

TABLE 1

| | | | | | RT-PCR |
|---|---|---|---|---|---|
| | | | | | Product |
| | | | | | size |
| | | SEQ | Antisense | SEQ | (in |
| Gene | Sense primer | ID | primer | ID | base |
| Name | (from 5′→3′) | NO: | (from 5′→3′) | NO: | pairs) |
| CD105 | CACTAGCCAGGTC | 1 | GATCTGCATGTTGT | 9 | 679 bp |
| | TCGAAGG | | GGTTGG | | |
| Tie-2 | CCTTAGTGACATT | 2 | GCAAAAATGTCCAC | 10 | 243 bp |
| | CTTCC | | CTGG | | |
| CD31 | GCTGTTGGTGGAA | 3 | GAAGTTGGCTGGAG | 11 | 700 bp |
| | GGAGTC | | GTGCTC | | |
| CD90 | CCCAGTGAAGATG | 4 | GACAGCCTGAGAGG | 12 | 185 bp |
| | CAGGTTT | | GTCTG | | |
| CD73 | CACCAAGGTTCAG | 5 | GTTCATCAATGGGC | 13 | 1007 bp |
| | CAGATCCGC | | GACCGG | | |
| Calponin | GAGTGTGCAGAC | 6 | CGAAGCCGGCCTTA | 14 | 671 bp |
| | GGAACTTCAGCC | | CAGA | | |
| α-SMA | CCAGCTATGTGAA | 7 | GTGATCTCCTTCTGC | 15 | 976 bp |
| | GAAGAAGAGG | | ATTCGGT | | |
| fsp1 | AGGAGCTACTGAC | 8 | TCATTGTCCCTGTTG | 16 | 102 bp |
| | CAGGGAGCT | | CTGTCC | | |

Table 1. Provided are the primers used for RT-PCR along with their sequence identifiers (SEQ ID NO:) and gene name.

### Example 1

### Emergence of Vasculogenic Progenitor Cells in the Course of EBS Maturation

Experimental Results

Emergence of vasculogenic progenitor cells in the course of EBs maturation—To follow the emergence of vasculogenic populations from spontaneously differentiating embryoid bodies (EBs) of human pluripotent cells (PSC), the present inventors used two models of differentiation using human embryonic stem cells and induced pluripotent stem cells. The cell lines used were as follows: for human embryonic stem cells the H9.2 and 16 cell lines were used; for induced pluripotent stem cells (iPSCs) the C3 and KTR13 were used. For characterization of vasculogenesis in CD105 positive precursors, differentiating EBs were analyzed for the expression of several combinations of characteristic endothelial and mural cell surface markers by flow cytometry or by RT-PCR of extracted RNA.

Dissociation of EBs into cells prior to cell sorting and flow cytometry analyses—EBs taken at any day from day 1-26 were dissociated by treatment with Collagenase B and DNAse I in order to prevent cell clumping and enable better dissociation of the EBs.

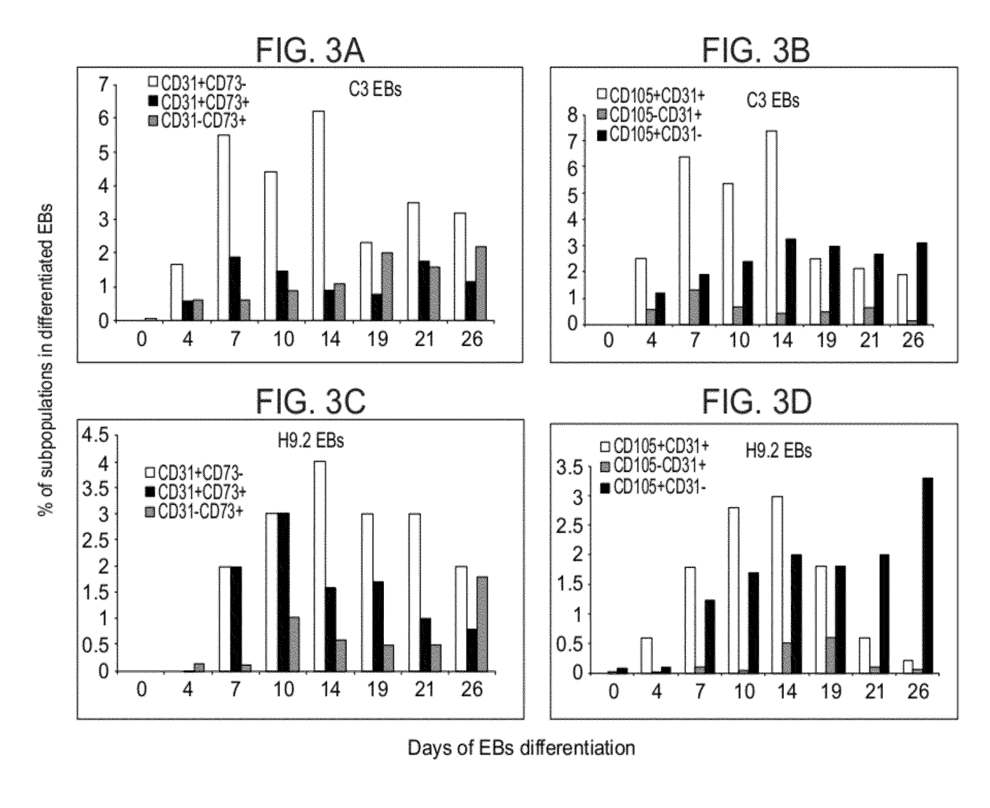

Characterization of CD105+ cells in EBs at various differentiation stages—Expression analysis of EBs at different days of differentiation demonstrated that CD105 expression was upregulated between days 4-26 in culture (FIG. 1A). Flow cytometry analysis revealed that while CD105+ cells constitute only 0.08% of the total EBs cells at day 1 of EBs differentiation, the fraction of CD105+ cells increases to about 4.1% (which includes the 3.5% of CD105+/CD31− and the 0.6% of CD105+/CD31+) of the total EBs cell population at day 26. Among the CD105+ cells there were two distinguished cell subsets: until day 14 the majority of CD105⁺ cells were composed of CD105+CD31+ endothelial cells (i.e., 3% on day 14) (FIGS. 2A-P and FIGS. 3A-D) with a small subset of non-endothelial CD105+CD31− population (i.e., 2%) (FIGS. 2A-J and FIGS. 3A-D). From day 17 onward the percentage of CD105+CD31+ endothelial cells as well as CD31 gene expression declined progressively coinciding with an increase in the percentage of CD105+CD31− non-endothelial subset (FIGS. 2K-P and FIGS. 3A-D).

Viability of isolated CD105+/CD31+ cells—To test the viability of the isolated CD105+ cells, the present inventors used the Trypan Blue assay. As shown in FIG. 37, the optimized dissociation protocol of EBs, which included Collagenase B and DNAse I, resulted in up to 2-10 fold increase in the apparent percentage of dissociated CD105+CD31⁺ cells (H9.2 n=30, 16 n=4, C3 n=12, KRTN n=3) and significantly increased the yield (FIG. 25A-B) of isolated cells with ≧95% viability. In contrast, the viability of CD105+/CD31+ cells isolated from EBs that were dissociated with 0.025%/0.01% trypsin/EDTA according to the method described in Levenberg S., et al., (2002, PNAS 99: 4391-4396), or in a non-enzymatic solution as described in Levenberg S., et al., (2010, Nature Protocols 5: 1115-1126) was only 65% (See FIG. 37). These results demonstrate that the dissociation step of the EBs determine the viability of the cells isolated from these EBs and shows the clear superiority of the dissociation method of the instant application over prior art methods.

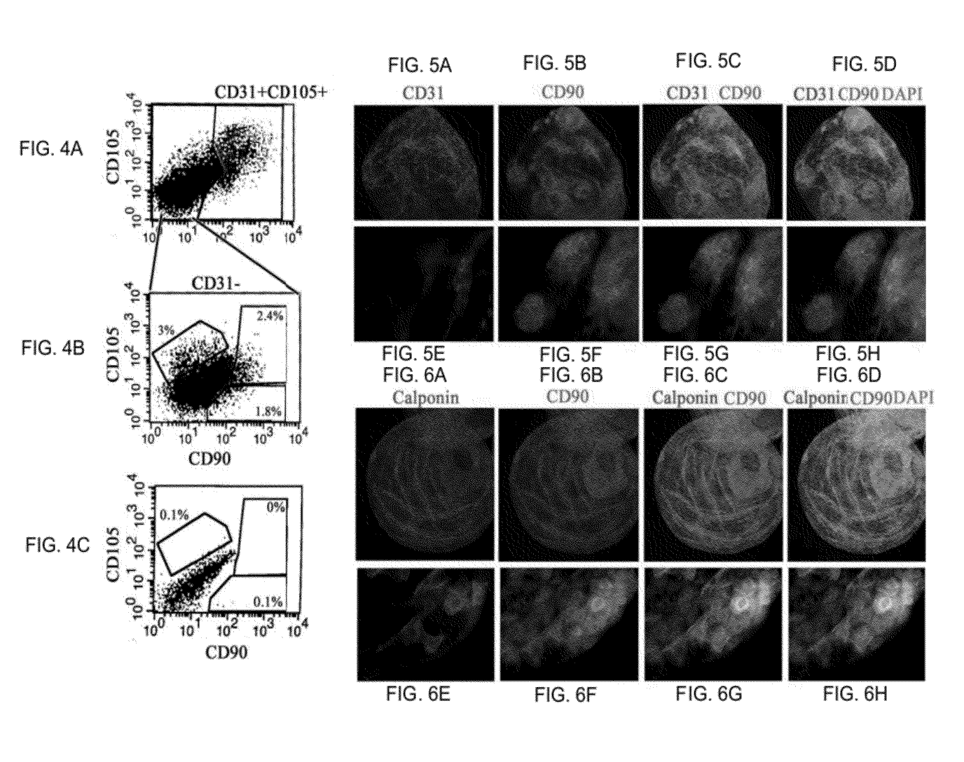

Characterization of the CD105+/CD31− cells isolated from EBs—Flow cytometry analyses of triple labeling of 19 days old dissociated EBs with antibodies against CD105, CD90 and CD31 revealed that within CD105⁺ CD31⁻ populations from both hESC as well as iPSC derived, about 2% co-expressed the mesenchymal stem cell marker CD90 (FIGS. 4A-C), which is also expressed by perivascular cells of blood vessels (Crisan M., et al., 2008, Cell Stem Cells 3: 301-313). The presence of CD90+CD31− clusters in 17 days old EBs was confirmed by immuno-fluorescence microscopy, wherein CD90⁺CD3⁻ were observed in proximity and aligning the CD31⁺ vascular network (FIGS. 5A-H). As illustrated in FIGS. 6A-H, the majority of CD90 positive cells co-expressed calponin, which is another characteristic marker of perivascular cells.

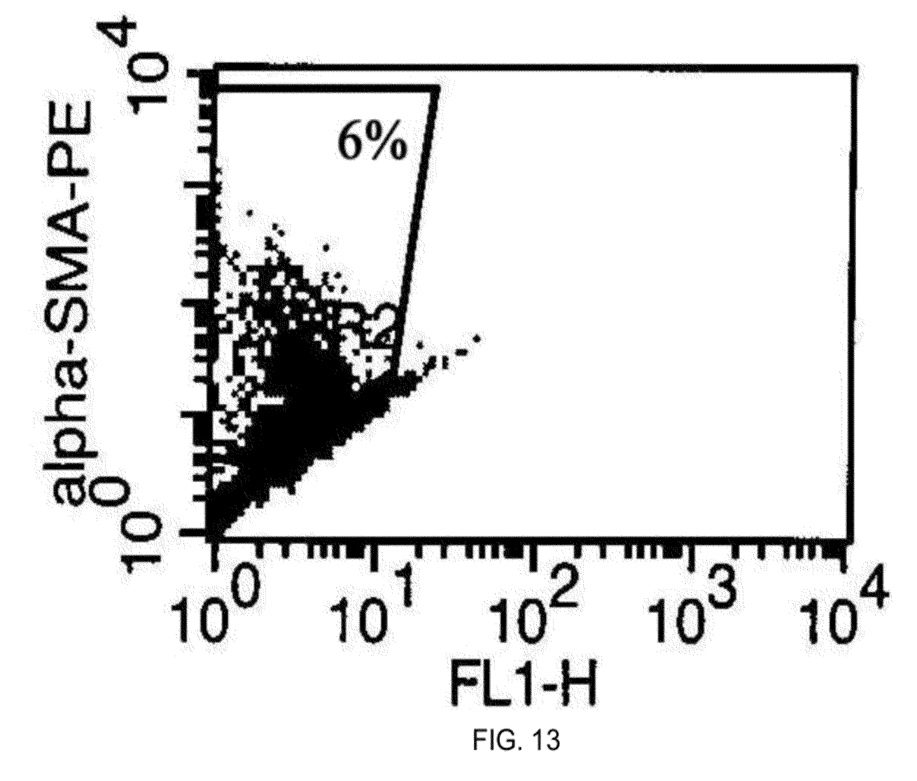

Altogether these results imply that in the course of vasculogenesis of developing EBs, a subset CD105⁺CD90⁺CD3⁻, which do not express α-SMA (FIG. 13 and data not shown), may represent a more immature perivascular progenitor cells with potential vasculogenic properties.

### Example 2

### Characterization of Pericyte-Like Progenitor Cells in Cultured CD105+/CD31− Cells

### Experimental Results

Identification of CD105+(positive) which are CD31− (negative) and αSMA− (negative)—When PSC-derived CD105⁺ cells from 17-19 days old EBs were sparsely-plated (3 cells/cm²) in endothelial cell (EC) growth medium 3 types of colonies could be identified within 7-10 days in culture: (1). CD31⁺ EC (endothelial cell) colonies; (2). Mixed CD31+ endothelial and CD31-αSMA+ cells with rare subset expressing both markers, and; (3). Non-ECs, of which the majority of cells were CD31⁻αSMA⁻ multilayered cells with a small subset of SMC (FIGS. 7A-I). The first two populations were previously described in various differentiation models of human ESC including spontaneously differentiating EBs (Levenberg S., et al., 2002, PNAS 99: 4391-4396), maturation on murine feeders (Wang Z., et al., 2007, Nat Biotechnol 25: 317-318; Yamahara K., et al., 2008, Plos One 3: e1666) or serum free, growth factors induced 2D culture (Daylon J., et al., 2010, Nat Biotechnol 28: 161-166). However, the third type of vasculogenic cells, αSMA− (αSMA negative) pericytic-progenitors, was never reported as an isolated population of cells.

Domination of the CD105+/CD31−/αSMA− cells in culture—Within 2-3 passages in culture the CD105+/CD31− subset dominated the mixed PSC-derived vasculogenic cultures exhibiting loosen cell-cell contacts in a multilayered hill and valley morphology FIGS. 7G-I, a cell morphology which was previously described in pericyte cultures from animal or human fetal or adult source (Majack R. 1987, The Journal of Cell Biology 105: 465-471; Crisan M., et al., 2008, Cell Stem Cells 3: 301-313) and which is typical of cultured pericytes as well as smooth muscle cells.

Pooled CD105+CD31− colonies (determined by flow cytometry analysis) emerging from PSC-derived CD105+ cells were cloned by limiting dilution at first passage and gave rise to single cell-derived colonies with similar morphology (data not shown) at clonal efficiency of 5+1.1% for H9.2 and 6+2% for iPSC C3.

Expansion of pericyte progenitor cells in culture up to 4200 fold increase within 7-8 passages—PSC-derived CD31⁻ pericyte-like cells could be efficiently expanded up to 2900 (H9.2) and 4200 (C3) fold increase (FIG. 8) in long term cultures up to 7-8 passages for 8 weeks, with a population doubling time (PDT)=108 hours between weeks 1-2 and a faster growth rate between weeks 3-5, at which the PDT was about 60 hours. After 7 weeks of culture pericytes gradually entered hypertrophic senescence (FIG. 9). Contact inhibition was not observable and adherent spherical clusters of multilayered cells were detached from the plastic at high densities (data not shown).

### Example 3

### Cultured Pericyte Progenitor Cells Exhibit a Unique Cell Surface Expression Pattern

Experimental Results

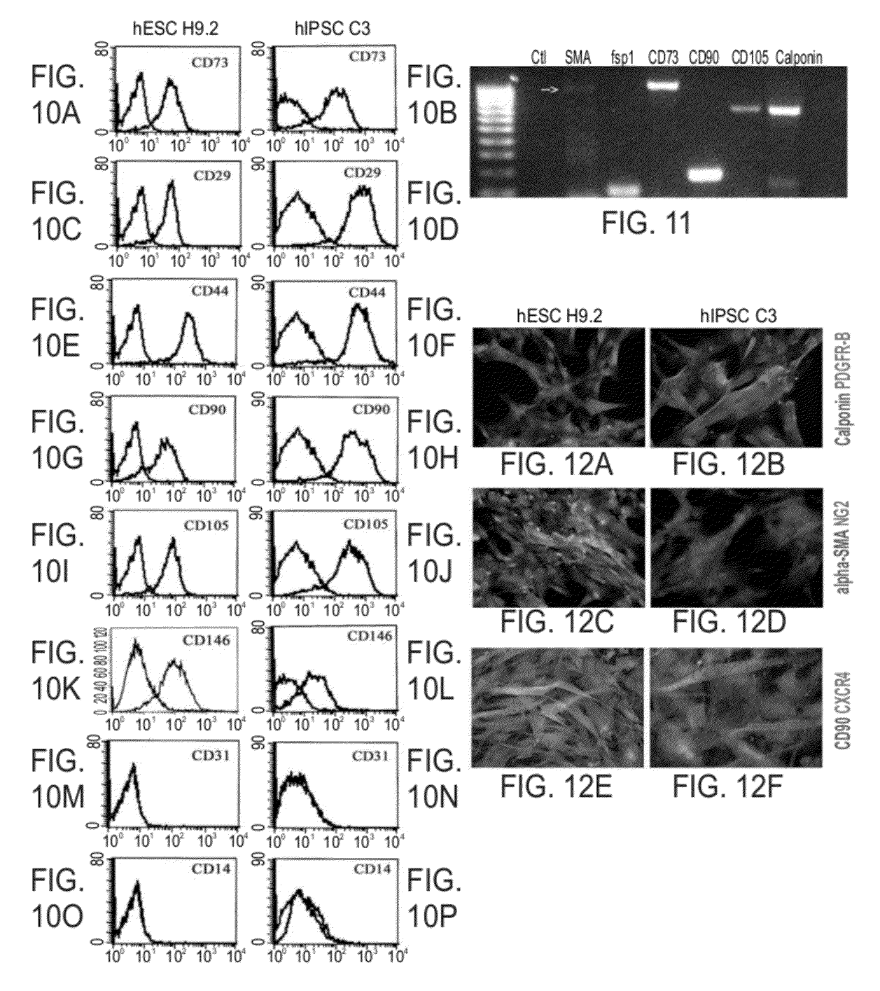

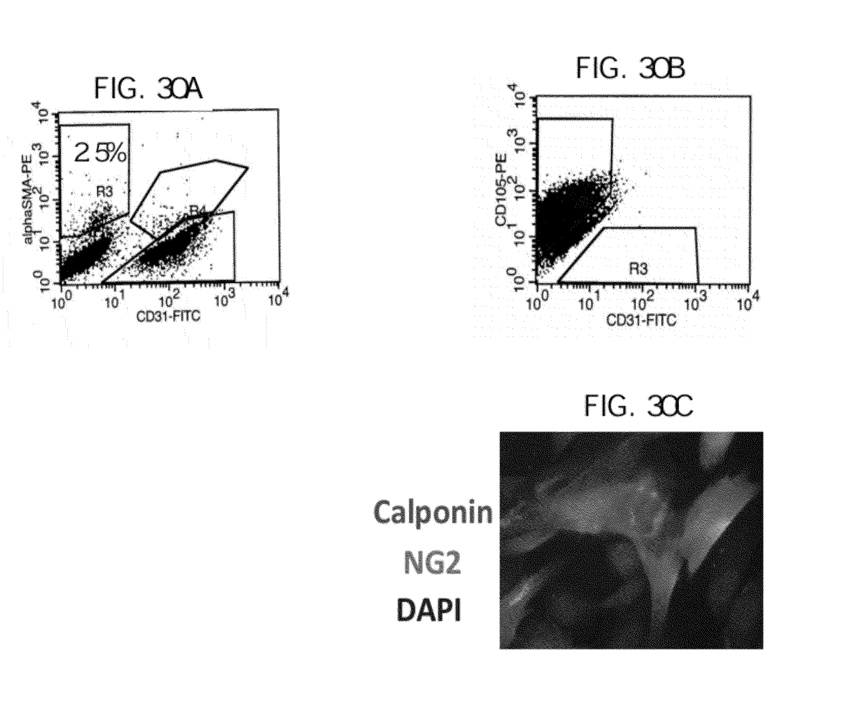

Antigens expressed by long-term cultured pericytes were assessed by flow cytometry, RT-PCR and immune-fluorescent microscopy. Analyses of expanded CD105⁺ CD31⁻ cells revealed that these cells presented over extended culture, a uniform and high expression of a comprehensive set of markers, which defines adult, fetal and embryonic tissue derived perivascular multipotent cells (Crisan M., et al., 2008, Cell Stem Cell 3:301-313), including CD105 (FIG. 30B), CD73, CD29, CD44, CD90 and CD146 (FIGS. 10A-P). However, in contrast to pericyte cells which were isolated from adult or fetal tissue, or from embryonic stem cells, at least 94% of the pericyte cells isolated herein were negative for expression of αSMA. Thus, from the first passage to senescence only about 6% of the cells were positive for α-SMA (FIG. 13 and FIG. 30A). Similar to adult derived pericytes and MSC, isolated and cultured CD105+CD31− cells did not express skeletal muscle cells markers (CD56 and E-cadherin), specific markers of endothelial cells (CD31, vW Factor, VE-Cadherin), hematopoietic markers (CD45, CD14, CD38) cells, or hemangioblast cell markers (CD34, CD133, Flk1, Flt-1) (FIGS. 10A-P, Table 2 hereinbelow, and data not shown). This was confirmed by RT-PCR at early and late passages of cultured pericytes (FIG. 11 and data not shown).

Regardless of cell passage, cultured pericytes were positive for identified perivascular markers including CD146 (FIGS. 10K-L), NG2 (FIGS. 12C-D), Calponin (FIGS. 11 and 12A-B) and PDGFR-β (FIGS. 12A-B), negative for the endothelial specific marker CD31 (FIGS. 10M-N), as well as for hematopoietic cell markers such as CD45 (data not shown) CD14 monocytic marker (FIGS. 10O-P).

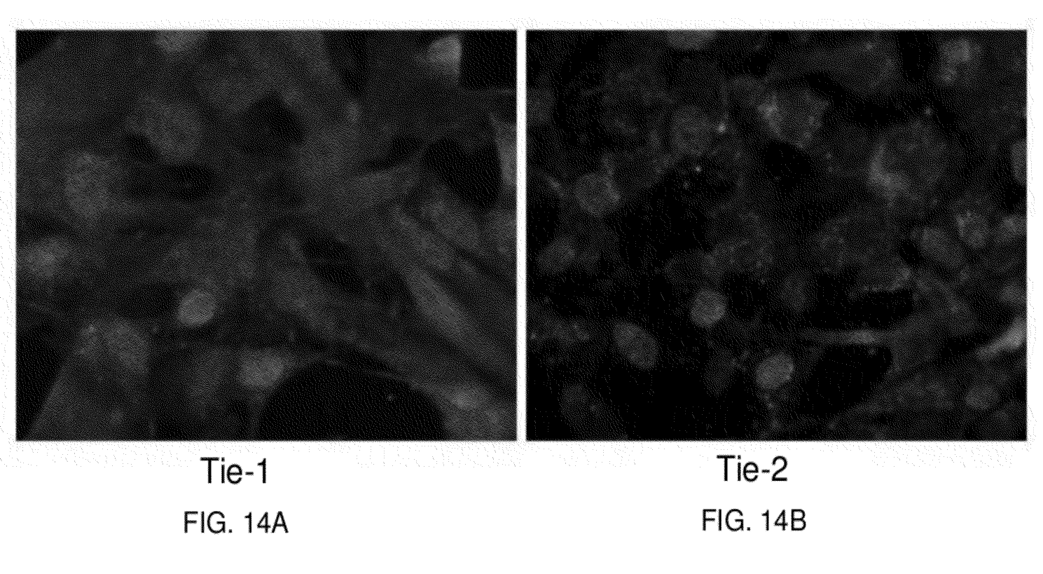

In accordance with their perivascular cell-like appearance, pluripotent stem cells (PSC) derived CD105+/CD31− cells demonstrated positive immuno-labeling for recognized pericytic markers including NG2, PDGFR-β, Calponin, CD90, Tie-1 and Tie-2 (FIGS. 12A-F, FIGS. 14A-B, FIGS. 29A-F, FIG. 30C and Table 2 below). Corresponding with the flow cytometry analyses (FIGS. 10A-P), a few α-SMA positive cells (up to 6%) were observed among cultivated pericyte-clusters (FIGS. 12C-D, red; and the faint band in RT-PCR analysis shown in FIG. 11).

TABLE 2
Expression profile of the isolated and cultured pericyte progenitor cells
and isolated endothelial progenitor cells isolated from embryoid bodies
according to the method of some embodiments of the invention.

| | | | | Crisan et al., | | Cho et al., |
|---|---|---|---|---|---|---|
| | | | | 2008 Cultured | Huard and | 2001, EBs- |
| | | | | pericytes after | Peault US | derived cells |
| Marker | EPC | Pericytes | MSC | 4-14 passages | 2007/0264239 | which are Flk1+ |
| Endothelial markers | | | | | | |
| CD31/PECAM-1 | + | − | − | | | |
| Tie-2 | + | + | − | | | |
| Tie-1 | + | + | ND | | | |
| VE-cadherin/CD144 | + | − | − | − | − | |
| CD105 | + | + | + | + | | + |
| UEA-1 | + | − | − | | | |
| vW Factor | + | − | − | − | − | |
| Ulex europaeus | + | − | ND | | − | |
| lectin (UEA-1) | | | | | | |
| Pericytes markers | | | | | | |
| NG2 | − | + | ND | + | + | |
| Calponin | | + | | | | |
| PDGFR-β | − | + | | + | | |
| (CD140b) | | | | | | |
| α-SMA | −** | −* | + | + | | |
| CD146 | + | + | + | + | + | |
| CD31 | | − | | − | − | − |
| Mesenchymal stem | | | | | | |
| cell markers | | | | | | |
| CD90 | − | + | + | + | | |
| CD44 | + | + | + | + | | |
| CD29 | ND | + | + | | | |
| CD73 | + | + | + | + | | |
| Hematopoietic cell | | | | | | |
| markers | | | | | | |
| CD45 | − | − | − | − | − | + |
| CD14 | − | − | − | | | |
| CD38 | − | − | − | | | |
| Alkaline | | | | + | | |
| phosphatase | | | | | | |
| Pax 7 | | | | − | | |
| Hemangbioblastic | | | | | | |
| markers | | | | | | |
| Flk1 | + | − | ND | | | + |
| CD34 | + | − | − | − | | |
| Flt-1 | + | − | | | | |
| CD133 | ND | − | ND | | + | |
| Skeletal Muscle | | | | | | |
| cell marker | | | | | | |
| CD56 | | − | | − | − | |
| E-cadherin | | − | | | | |

Table 2: Provided are the expression patterns of the isolated and cultured pericyte progenitor cells and endothelial progenitor cells (EPCs) according to the method of some embodiments of the invention from any passage beginning from passage 1 to senescence (passage 5 for ECs and passage 8 for pericytes). Mesenchymal stem cells (MSC) were derived from human adult adipose tissue. The expression patterns were revealed by flow-cytometry, RT-PCR and immunolabeling experiments. The expression pattern of cells isolated from EBs by Cho et al., (Blood, 98: 3635-3642) or from multiple adult or embryonic tissues/organs by Crisan et al., 2008 (Cell Stem Cell 3: 301-313) or US 2007/0264239 (Huard J and Peault B M) are also provided for comparison.
“+” positive expression (i.e., express the indicated marker);
“−” negative expression (i.e., do not express the indicated marker);
ND = not determined.
*Note that the majority of the isolated pericyte progenitor cells are αSMA− (negative), while minor fractions (based on immuno staining) of PSC-derived endothelial cells (ECs) and pericyte progenitor cells (up to 6%) express α-SMA.
**most of the endothelial progenitor cells are αSMA− except a minor subset.

### Example 4

### PSC-Derived Pericytes and Endothelial Cells Assemble to Generate Human Vascular Network

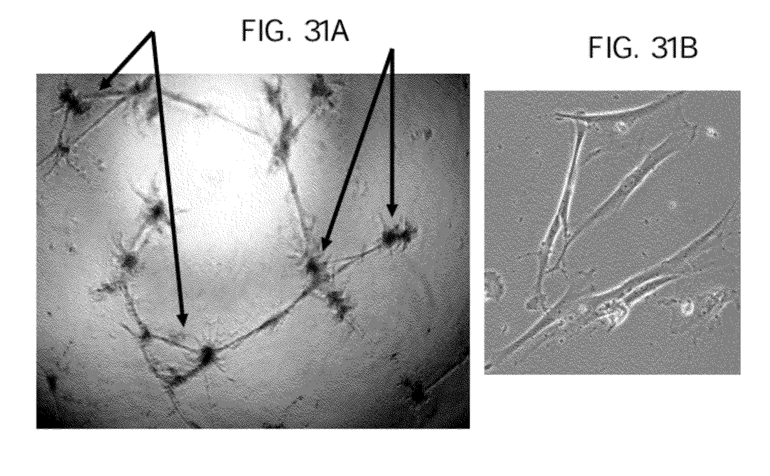

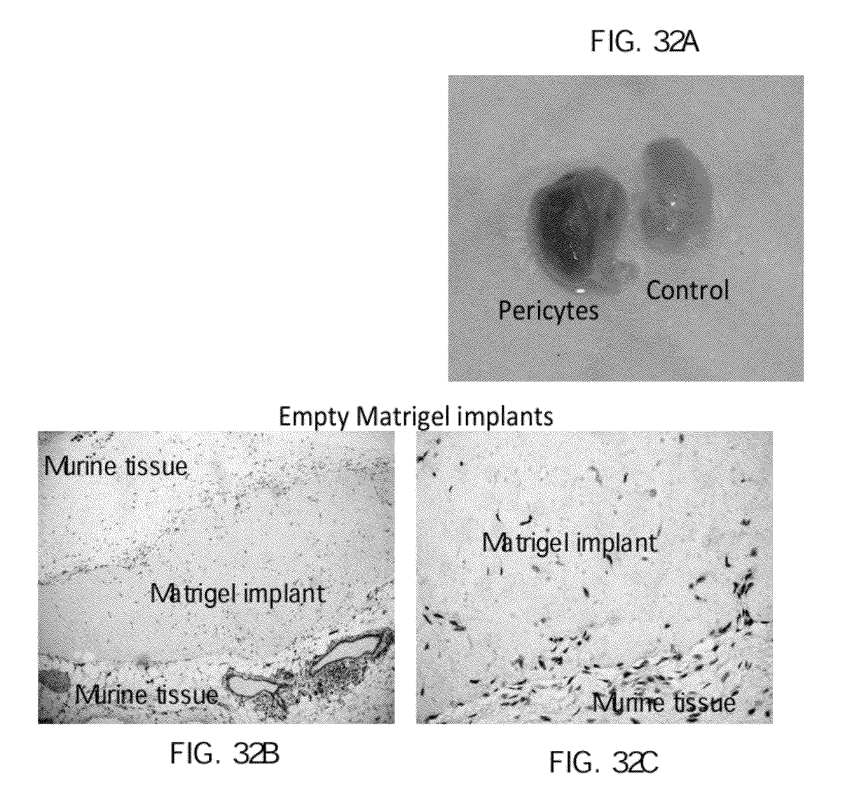

Vasculogenic potential of pericytes was initially evaluated in vitro. Although tube formation on Matrigel is a common feature of endothelial cells, it has been previously demonstrated that fetal tissue derived-a SMA positive pericytes can actively create tubular networks (Bagley R G, et al., Cancer Res. 2005 Nov. 1; 65(21):9741-50). The PSC-derived pericytes generated according to the method of some embodiments of the invention formed short, intricate tubular structures on Matrigel within 2 hours (FIG. 15A) with sprouting clusters in between (FIG. 31A), while HUVEC (data not shown) or PSC-derived CD31⁺ endothelial cells re-arranged in linear, asymmetric tube networks that regenerated within 12 hours (FIG. 15B).

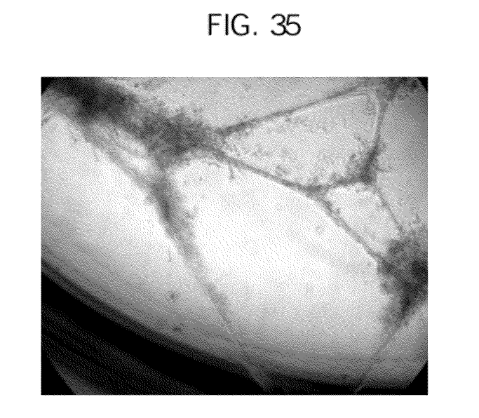

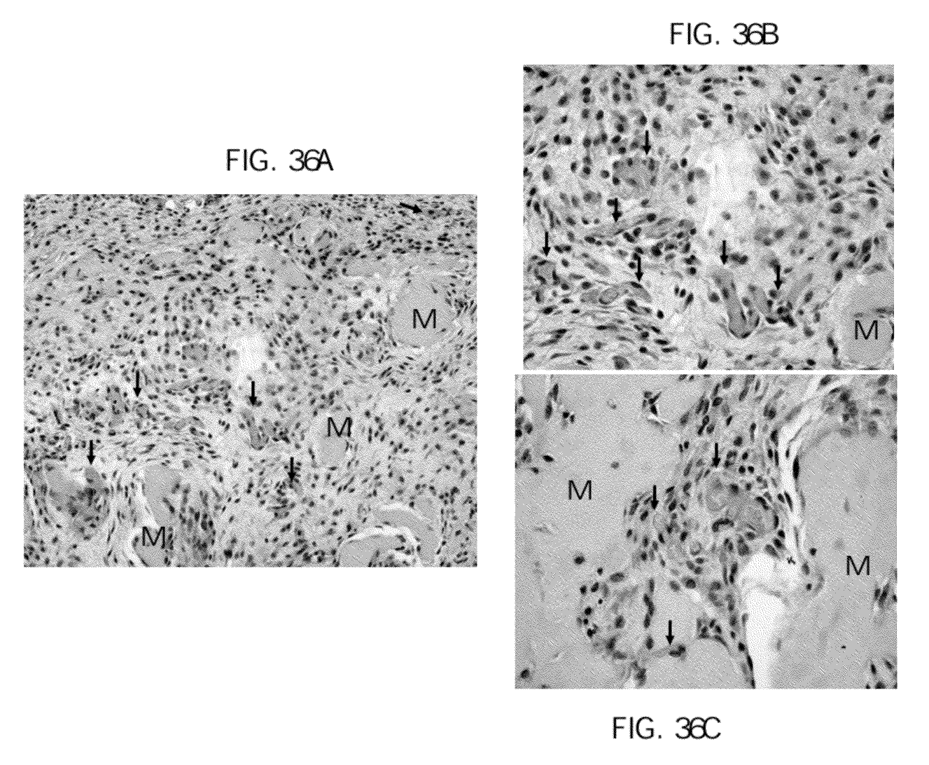

A characteristic feature of pericytes in developing and adult tissue is their ability to form cell-cell contact with endothelial cells to form a vascular network. When carboxyfluorescein diacetate succinimidyl ester (CFSE)-labeled pericytes were co-cultured with H9.2 hESC-derived CD31⁺ endothelial cells on Matrigel both cells were assembled to form thicker tubular networks (FIGS. 16A-H and FIG. 35). It has been previously demonstrated that the presence of murine or human adult tissue derived mesenchymal precursors are essential for de novo generation and stabilization of vascular networks using immunodeficient murine experimental models. Therefore, the vasculogenic potential of PSC-derived CD31⁻CD105⁺ pericytes was studied in vivo, using Matrigel implant assay. Pericytes and endothelial cells, either HUVEC or PSC-derived endothelial cells, were mixed in Matrigel and injected subcutaneously. Hematoxylin/Eosin staining revealed that empty control Matrigel implants (FIGS. 32B and 32C) or implants with HUVEC (data not shown) or PSC-derived CD31⁺ cells only did not contain microvessels (FIG. 17A-E and FIGS. 32A-C). Implants with only pericytes exhibited infiltration of few murine vessels (FIG. 17C and FIGS. 33A and 33B). However, when pericytes were mixed with either HUVEC (FIG. 17D) or PSC-derived EC (FIG. 17E and FIGS. 36A-C) and implanted in a ratio of 50:50, a robust generation of evenly distributed vascular network was detected inside the implant by Hematoxylin/Eosin staining. The presence of luminal murine erythrocytes and nucleated leukocytes (FIG. 17D-E and FIGS. 36A-C) indicate that the newly formed human blood vessels anatomized with the host vasculature and perfused with murine blood within 1 week.

The isolated pericytes and endothelial cells express human MHC class I—Flow cytometry analysis (data not shown) and immune-fluorescence microscopy revealed that cultured PSC-derived pericytes expressed cell surface and cytoplasmic human MHC Class I (FIG. 18A and data not shown) and in accordance were identified as MHC Class I⁺ CD31⁻ within the implants. As seen in FIGS. 18B-E, the majority of the luminal blood vessel cells within the implants were positive for labeling with specific antibodies against human (and not mouse) endothelial markers CD31 or CD34 (data not shown), which co-expressed von-Wilebrand's factor (FIG. 18D, vW factor). Pericytes were seen in proximity and aligning CD31⁺ or CD34⁺ human vascular structures (FIGS. 18B-E).

Of notice, efficient generation of chimeric human vasculogenic structures was detected when the implants were devoid of angiogenic factors such as VEGF or/and bFGF (data not shown).

### Example 5

### Pluripotent Stem Cells-Derived Pericytes are Multipotent and Capable of Differentiation into Bone, Cartilage, Far and Muscle Cells

To examine the potential of the isolated and cultured pericyte progenitor cells according to some embodiments of the invention to form mesenchymal tissues, including fat, cartilage, myogenic, and bone, short-term and long-term cultured pericytes were used from passages 1-8, wherein at passage 9 cells entered senescence.

Differentiation to osteoblasts in vitro—Upon appropriate inductive experimental conditions (in the presence of an osteogenic medium), PSC-derived pericytes (H9.2 and C3) exhibited uniform osteogenic differentiation (FIGS. 19A and 19B). Mineral deposits were formed (FIG. 19A) with massive calcium content as detected by alizarin red staining (FIG. 19B). PSC-derived pericytes maintained their osteogenic potential in vitro throughout the whole culture period between passages 2-9 (data not shown). This fast and efficient differentiation into osteoblasts in culture is remarkable as compared to differentiation of pericytes described by others (e.g., as described by Crisan et al., 2008), regardless of cell passage.

Osteogenic differentiation in vivo—PSC-derived pericytes were cultured in osteogenic medium, removed after 3 or 14 days of osteogenic stimulation, mixed with Matrigel and implanted subcutaneously into NOD/SCID immune deficient mice for evaluation of osteogenesis by ectopic model in vivo. Hematoxylin and Eosin staining revealed appearance of mineral deposits in vivo already after 3 days of stimulation in osteogenic medium (FIG. 19C). Increased amount of deposits within implant is seen when PSC-derived pericytes were stimulated for 14 days (FIG. 19E). The amount of calcium was noticeably increased with longer cultivation period (FIG. 19F) compared to short stimulation of PSC-derived pericytes (FIG. 19D) in vitro as detected by alizarin red staining.

Differentiation to chondrocytes—When PSC-derived pericytes cell pellets were cultivated in chondrogenic medium, spherical structures were formed and showed positive von-Kossa staining (data not shown).

Differentiation to adipocytes—Adipogneic potential of PSC-derived pericytes was tested in vitro by cultivation in adipogenic medium for 4 weeks. The majority of cultured PSC-derived pericytes (about 90%-95%) contained lipid vesicles positive for Oil red staining (H9.2 derived-pericytes, FIG. 20A; iPSC C3-derived pericytes, FIG. 20B). PSC-derived pericytes maintained their adipogenic potential in vitro throughout the whole culture period between passages 2-9 (data not shown).

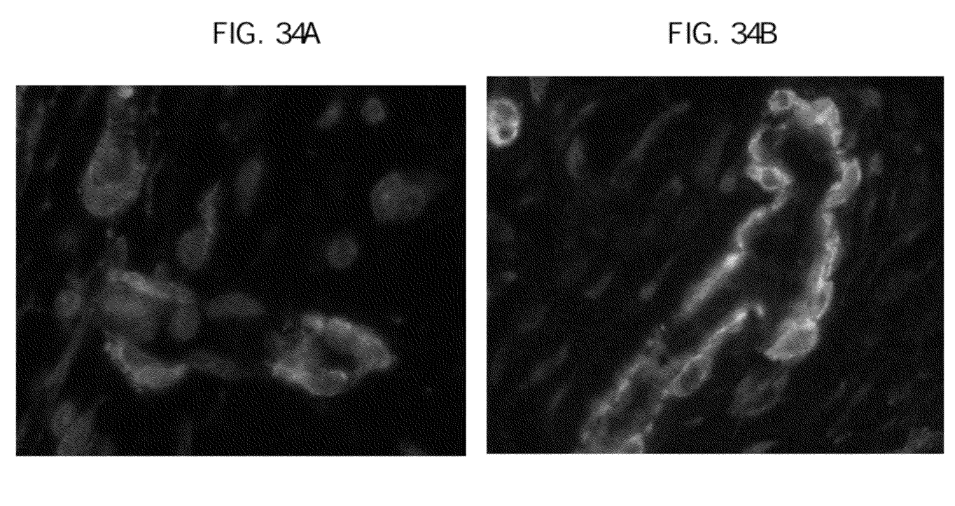

Differentiation to myogenic cell lineage and generation of myotube—Myogenic differentiation was seen in Matrigel implants in which all transplanted α-SMA negative pericytes further differentiated in vivo within the Matrigel implant toward smooth muscle cells, expressing α-SMA (FIGS. 33C and 33D) which were able to assemble with newly formed murine blood vessels within the implant (FIGS. 34A and 34B). When stimulated with myogenic medium (DMEM media supplemented with 2% horse serum), the pericyte progenitor cells fused to form thick multinucleated myotubes within 7 days in vitro (FIG. 38, see arrows pointing at nuclei, and data not shown).

### Example 6

### Co-Derivation of Vasculogenic Pericytes and Endothelial Cells Form PSC-Derived Spontaneously Differentiating Embryoid Bodies

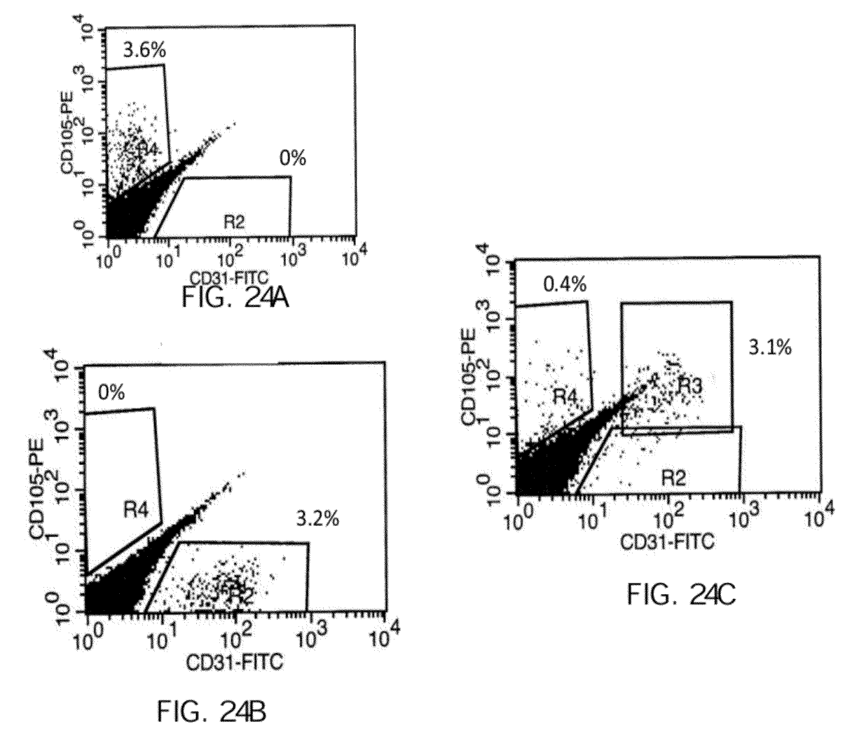

For co-derivation of vasculogenic populations of EPC and pericytes form PSC (FIG. 23A), either human ESC or human iPSC, spontaneously differentiated embryoid bodies (EBs) at days 7-26 were dissociated to single cell suspensions with Collagenase B and DNAse I. Dissociated cells were then immuno-labeled with either PE or FITC-conjugated antibodies which specifically recognize CD105 or CD73 (FIGS. 2A-P and FIGS. 39A-F), for further sorting or by microbeads-conjugated antibodies for further magnetic separation (FIG. 23A). Isolated cells at passage 0 (cultured immediately post isolation) were composed of two subsets: CD31+CD105+ or CD31+CD73+ endothelial cells and CD105+CD31− or CD73+CD31− pericytes (FIG. 23B, FIGS. 24A-C, FIGS. 2A-P and FIGS. 39A-F). After further expansion in endothelial cell M-199 culture media, a second isolation step was preformed for separation of endothelial cells from pericytes (FIGS. 26D and 26E) for further expansion of both populations from passage 1. Antibodies against CD31 or UEA-1-FITC lectin were used for isolation of endothelial cell population (CD31+UEA-1+CD105+ or CD31+UEA-1+CD73+) for further cultivation (positive fraction, FIG. 23D and FIG. 26C). Negative cell fraction CD105+CD73+CD31− consisted of pericytes at passage 1 post second isolation step (negative fraction, FIG. 23E and FIG. 26B).

### Example 7

### Cultivation and Expansion of PSC-Derived Endothelial Cells

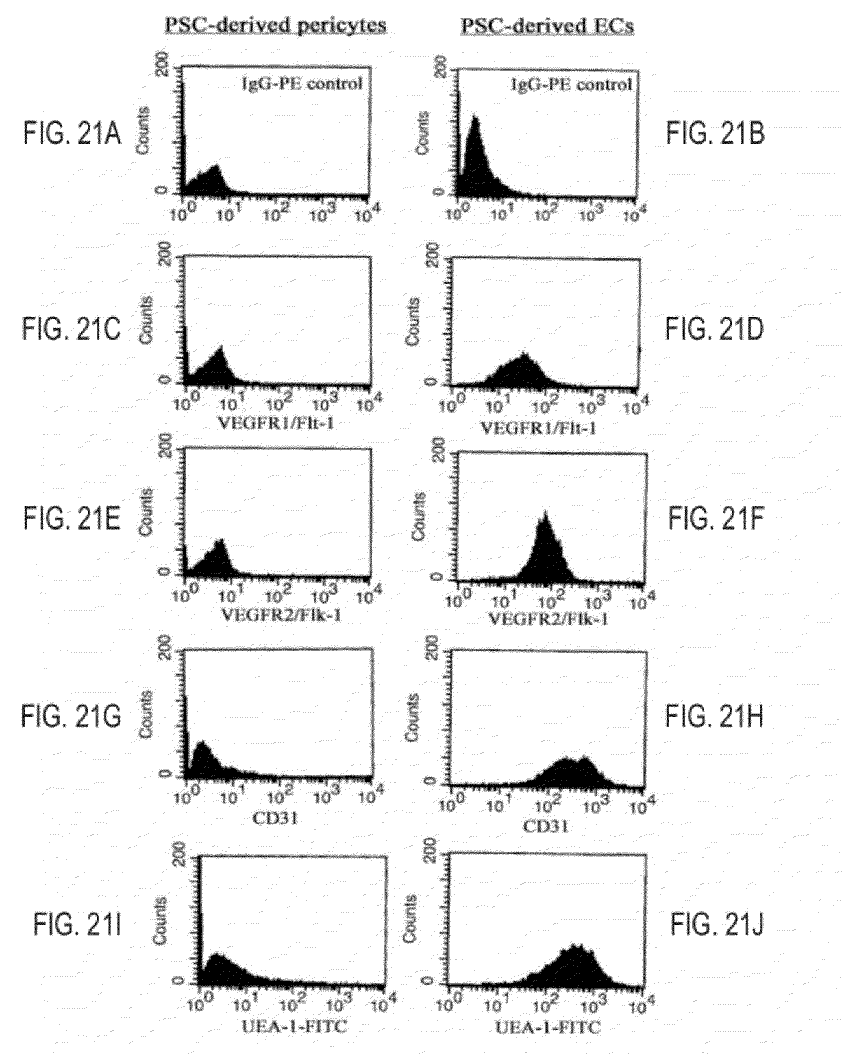

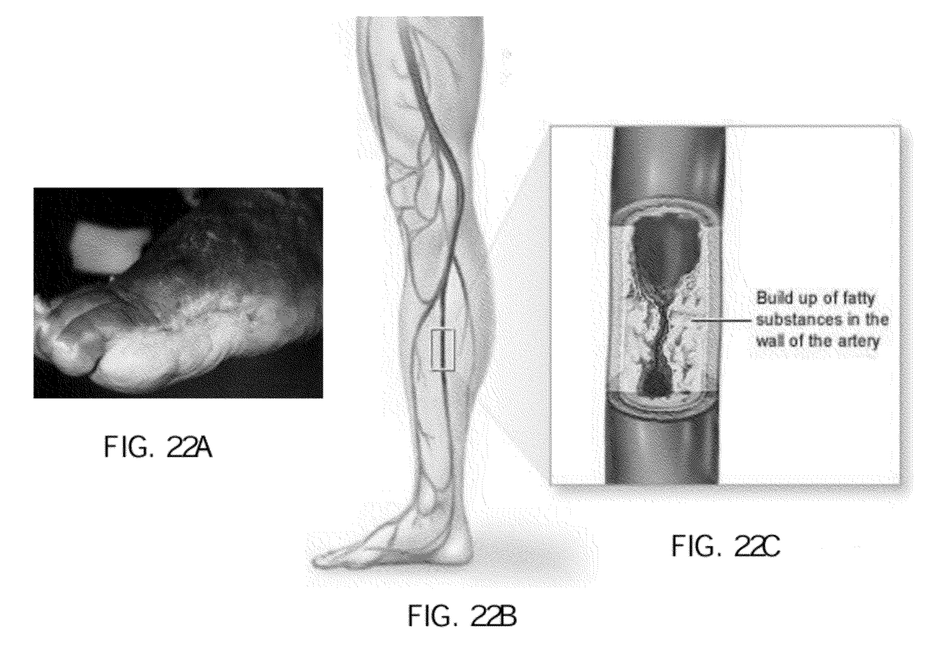

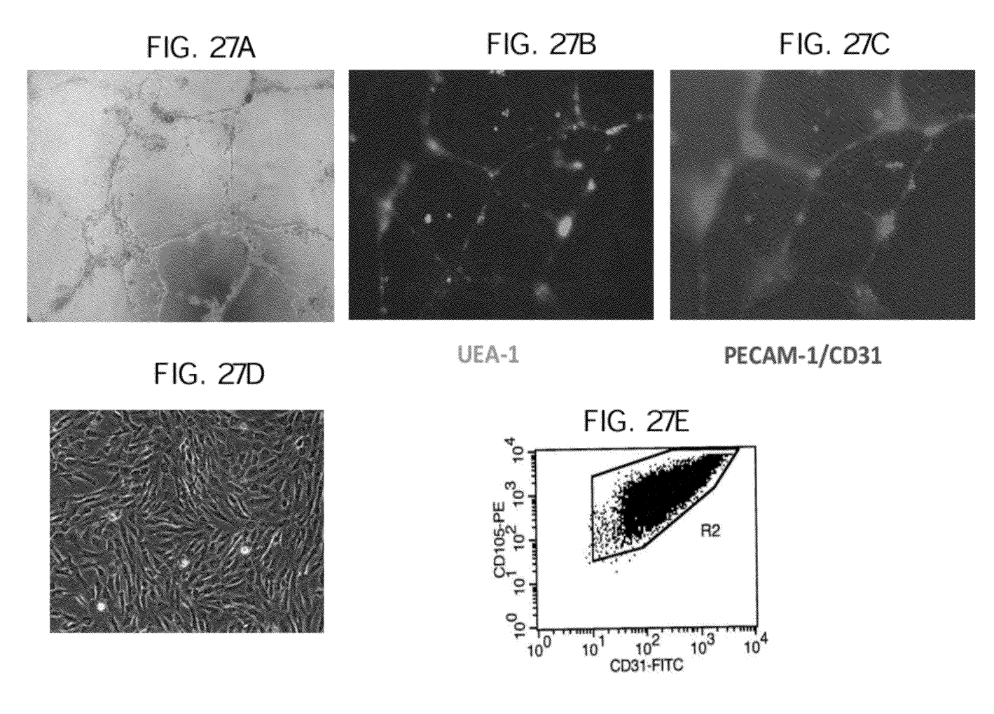

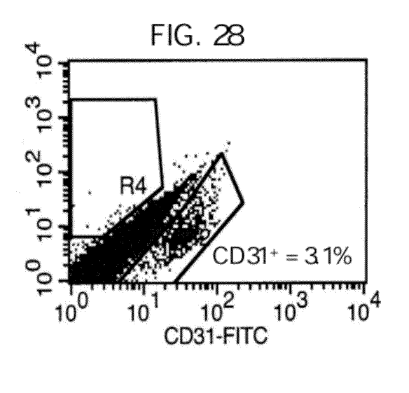

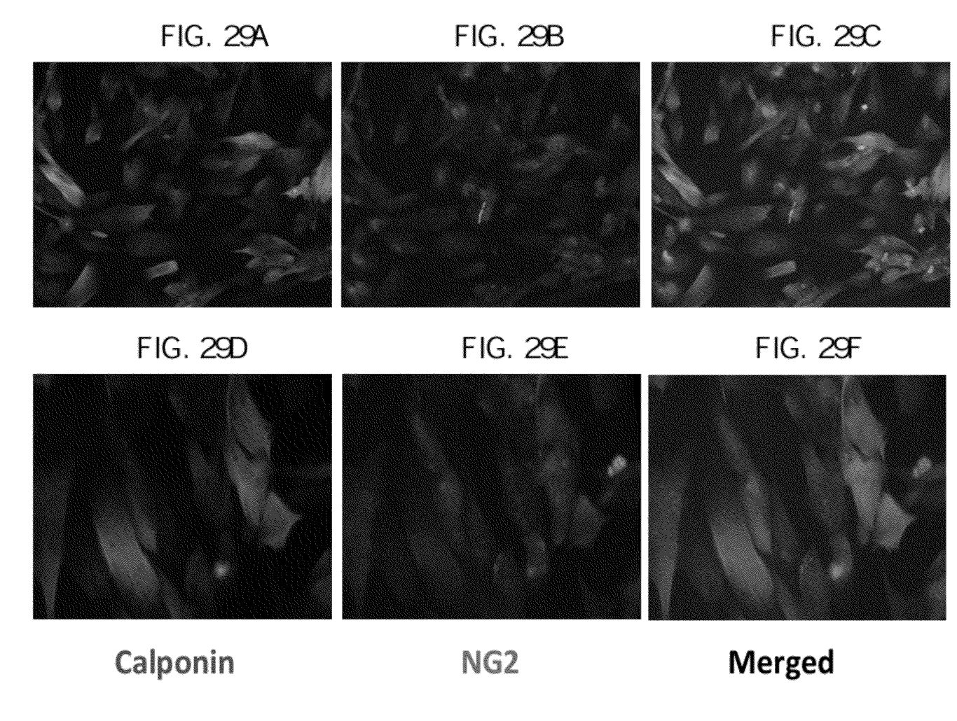

PSC-derived EPCs (CD31+CD105+ or CD31+CD73+), which were isolated based on expression of CD105 or CD73, were further cultivated after a second isolation step at passage 1 up to passage 5 in human fibronectin-coated or gelatin coated culture dishes in EC M-199 growth medium or in EBM-2 medium supplemented with 10 μM TGF-β inhibitor SB431542 (Tocris). Expanded endothelial cells exhibited characteristics of adult tissue counterparts in vitro and in vivo expressing: VEGFR1 (FIG. 21D), VEGFR2 (FIG. 21F), CD31 (FIG. 21H), UEA-1 (FIG. 21J), vW Factor (FIG. 26C) and VE-Cadherin (FIG. 26E). CD105+PSC-derived endothelial cells formed tube networks when seeded on Matrigel (FIG. 27A), which were positive for UEA-1 (green, FIG. 27B) or CD31 (red, FIG. 27C). Cobbelstone forming cells were seen in culture dishes until passage 4-5 (FIG. 27D). Expanded PSC-derived endothelial cell maintained CD31 as well as CD105 expression throughout the whole culture period (FIG. 27E).

The present inventors have uncovered novel methods of isolating pericytes from pluripotent stem cells by developing a unique approach to trace the potential emergence of pericytes alongside with development of endothelial and SMC based on common (CD105, α-SMA) and specific (CD31, VE-Cadherin, UEA-1) vascular cell markers. Thus, using the teachings of the invention the inventors have identified a novel population of cells positive for recognized markers of pericyte, including NG2, and PDGFR-β, and CD146 along with other markers of mesenchymal stem cells, but negative for α-SMA. The unique expression pattern of the isolated population of cells implies that these cells are more primitive ancestors within pericyte hierarchy. In accordance, cultured pericytes exhibited angiogenic characteristics in vitro and in vivo and maintained the ability to differentiate into chondrogenic, myogenic and adipogenic cell lineages upon sustained expansion (e.g., even following 7-9 passages in culture). In particular, PSC-derived pericytes exhibited highly efficient and rapid osteogenic response in vitro compared to their MSC counterparts as well as ectopic bone formation after implantation into NOD/SCID mice. These results demonstrate an efficient derivation of multipotent mesodermal pericytic-precursors from PSCs.

In addition, the invention provides a method for efficient co-derivation of the two blood vessel cellular components, pericytes and endothelial cells, from a single pluripotent cell source. The method is based on two isolation steps, wherein a single specific cell surface marker is used in each step. Initially, CD105⁺ cells are isolated from embryoid bodies by magnetic separation or fluorescent sorting. The isolated CD105⁺ population is then cultured in endothelial cell media, which gives rise to two distinguished cell populations: CD105⁺CD31⁺UEA-1⁺ endothelial cells and CD105⁺ CD31⁻UEA-1⁻ cells. In the second step, all cultured CD105⁺ cells are incubated with either UEA-1 or CD31⁺ antibodies to distinguish between CD31⁺ endothelial population and CD31⁻ cells.

Robust expansion of mature vasculogenic cells, derived from pluripotent cells with the potential to achieve sufficient cell numbers for clinical transplantation was not demonstrated before. Equivalent models are much more expensive and complicated, since multipotent progenitors are first isolated from differentiating stem cells to be then used for derivation of mature target cells. Existing techniques require the addition of large amounts of expensive growth factors, which are essential for directed differentiation of the isolated progenitor cells. In addition, the separation methods are based on multi-marker identification in order to isolate both progenitor cells and mature populations. In contrast, the novel method of some embodiments of the invention uses a simple and inexpensive culture system of spontaneously differentiated embryoid bodies, from which target mature vasculogenic populations are isolated based on single cell surface marker.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

Armulik et al., 2005, Circulation Research, 97: 512-523;
Bergers and Song, 2005, Neuro-Oncology, 2005, 7: 452-464;
Farrington-Rock et al., 2004, Circulation, 110:2226-2232;
Dellavalle et al., 2007, Nature Cell Biology, 9: 255-267;
Crisan M., et al. Cell Stem Cell 3, 301-313, 2008;
Levenberg S., Nat Protocols, 5: 1115-1118, 2010;
Goldman 0., et al., 2009, Stem Cells 27: 1750-1759;
Daylon J., Nature Biotechnology, advanced online publication, 17 Jan. 2010;
Bai., et al., 2010, Journal of Cellular Biochemistry 109: 363-374;
Levenberg S., et al. PNAS USA 2002, 99:4391-96;
Bryan and D'Amore 2008 Methods in Enzymology, 443: 315-331;
Cho SK., et al. 2001, Blood, 98: 3635-3642;
Lindskog H., et al. 2006, Arteriosclerosis, Thrombosis, and Vascular Biology, 2006, 26:1457-1464;
Brachvogel et al., 2005, Development 132: 2657-2668;
Corselli et al., 2010, Arterioscler Thromb. Vasc. Biol. 2010, 30: 1104-9;
Bagley R G., Cancer Res. 2005, 65: 9741-50;
US Patent Application 2007/0264239 (Huard J and Peault B M);
Barbery T., et al., 2005, PLoS Medicine, 2: 554-560;

1. A method of isolating a pericyte progenitor cell from embryoid bodies, comprising:
(a) isolating from the embryoid bodies using an anti CD105 antibody and/or an anti CD73 antibody cells which are CD105+ and/or CD73+, respectively, to thereby obtain CD105+, CD73+ and/or CD105+/CD73+ cells, and;
(b) culturing said CD105+, CD73+ and/or CD105+/CD73+ cells,
thereby isolating the pericyte progenitor cell from the embryoid bodies.

2-5. (canceled)

6. A method of co-derivation of pericyte and endothelial progenitor cells, comprising:
(a) isolating from embryoid bodies using an anti CD105 antibody and/or an anti CD73 antibody cells which are CD105+ and/or CD73+, to thereby obtain CD105+, CD73+ and/or CD105+/CD73+ cells;
(b) isolating a CD31+/UEA-1+/Ve-cadherin+ cells from said CD105+, CD73+ and/or CD105+/CD73+ cells, to thereby isolate the endothelial progenitor cells;
(c) isolating CD31− cells from said CD105+, CD73+ and/or CD105+/CD73+ cells, to thereby isolate the pericyte progenitor cells;
thereby co-derivation of pericyte and endothelial progenitor cells.

7. (canceled)

8. The method of claim 6, further comprising:
culturing said CD31+/UEA-1+/Ve-cadherin+ cells, to thereby expand said endothelial progenitor cells.

9. The method of claim 6, further comprising:
culturing said CD31− cells, to thereby expand said pericyte progenitor cells.

10. The method of claim 1, further comprising passaging said CD105+, CD73+ and/or CD105+/CD73+ cells for at least 2 passages to thereby expand a population of pericyte progenitor cells.

11. The method of claim 1, further comprising enriching said cells for CD105+/CD31−, CD73+/CD31− and/or CD105+/CD73+/CD31− cells.

12. The method of claim 11, wherein said enriching is effected by depleting CD31+ cells from said CD105+, CD73+ and/or CD105+/CD73+ cells.

13. (canceled)

14. An isolated pericyte progenitor cell having a CD105+/CD31−/αSMA−/CD133−/Flk-1−/CD34−/NG2+/CD146+, a CD73+/CD31−/αSMA−/CD133−/Flk-1−/CD34−/NG2+/CD146+ or a CD105+/CD73+CD31−/αSMA−/CD133−/Flk-1−/CD34−/NG2+/CD146+ signature.

15. (canceled)

16. An isolated population of cells comprising at least 85% of the pericyte progenitor cell of claim 14.

17. A cell culture comprising a culture medium and the isolated pericyte progenitor cell of claim 14.

18. A method of generating osteoblast cells, comprising culturing the isolated pericyte progenitor cell of claim 14, in a culture medium which comprises β-glycerol-phosphate, Dexamethasone and ascorbic acid, thereby generating the osteoblast cells.

19. A method of generating adipocyte cells, comprising culturing the isolated pericyte progenitor cell of claim 14, in a culture medium which comprises IBMX (3-isobutyl-1-methylxanthine), Dexamethasone and insulin, thereby generating the adipocyte cells.

20. A method of generating chondrocyte cells, comprising culturing the isolated pericyte progenitor cell of claim 14, in a culture medium which comprises dexamethasone, ascorbic acid and TGFβ3, thereby generating the chondrocyte cells.

21-27. (canceled)

28. The isolated pericyte progenitor cell of claim 14, wherein the isolated pericyte progenitor cell is capable of differentiation into at least two cell lineages of the cell lineages selected from the group consisting of osteoblasts, chondrocytes, myobloasts and apipocytes.

29-32. (canceled)

33. The isolated pericyte progenitor cell of claim 28, wherein said differentiation into at least two cell lineages is maintained at any passage in culture from passage 1 to senescence.

34-36. (canceled)

37. The method of claim 1, wherein said culturing comprises about 7-9 passages.

38-47. (canceled)

48. A pharmaceutical composition comprising the isolated pericyte progenitor cell of claim 14, and a therapeutically acceptable carrier.

49. A method of treating a pathology requiring vascular tissue regeneration and/or repair, comprising administering to a subject having the pathology the isolated pericyte progenitor cell of claim 14, thereby treating the pathology.

50-52. (canceled)

53. A method of generating myoblasts, comprising culturing the isolated pericyte progenitor cell of claim 14, in a culture medium which comprises horse serum, thereby generating the myoblasts.

54. A method of generating smooth muscle cells in vivo, comprising implanting the isolated pericyte progenitor cell of claim 14 in a subject in need thereof, thereby generating the smooth muscle cells in vivo.

55. (canceled)


SEQUENCE LISTING (19 sequences)

SEQ ID NO: 1 - DNA, 20 bp, Artificial sequence
  Single strand DNA oligonucleotide
cactagccaggtctcgaagg

SEQ ID NO: 2 - DNA, 18 bp, Artificial sequence
  Single strand DNA oligonucleotide
ccttagtgacattcttcc

SEQ ID NO: 3 - DNA, 19 bp, Artificial sequence
  Single strand DNA oligonucleotide
gctgttggtggaaggagtc

SEQ ID NO: 4 - DNA, 20 bp, Artificial sequence
  Single strand DNA oligonucleotide
cccagtgaagatgcaggttt

SEQ ID NO: 5 - DNA, 22 bp, Artificial sequence
  Single strand DNA oligonucleotide
caccaaggttcagcagatccgc

SEQ ID NO: 6 - DNA, 24 bp, Artificial sequence
  Single strand DNA oligonucleotide
gagtgtgcagacggaacttcagcc

SEQ ID NO: 7 - DNA, 23 bp, Artificial sequence
  Single strand DNA oligonucleotide
ccagctatgtgaagaagaagagg

SEQ ID NO: 8 - DNA, 22 bp, Artificial sequence
  Single strand DNA oligonucleotide
aggagctactgaccagggagct

SEQ ID NO: 9 - DNA, 20 bp, Artificial sequence
  Single strand DNA oligonucleotide
gatctgcatgttgtggttgg

SEQ ID NO: 10 - DNA, 18 bp, Artificial sequence
  Single strand DNA oligonucleotide
gcaaaaatgtccacctgg

SEQ ID NO: 11 - DNA, 20 bp, Artificial sequence
  Single strand DNA oligonucleotide
gaagttggctggaggtgctc

SEQ ID NO: 12 - DNA, 19 bp, Artificial sequence
  Single strand DNA oligonucleotide
gacagcctgagagggtctg

SEQ ID NO: 13 - DNA, 20 bp, Artificial sequence
  Single strand DNA oligonucleotide
gttcatcaatgggcgaccgg

SEQ ID NO: 14 - DNA, 18 bp, Artificial sequence
  Single strand DNA oligonucleotide
cgaagccggccttacaga

SEQ ID NO: 15 - DNA, 22 bp, Artificial sequence
  Single strand DNA oligonucleotide
gtgatctccttctgcattcggt

SEQ ID NO: 16 - DNA, 21 bp, Artificial sequence
  Single strand DNA oligonucleotide
tcattgtccctgttgctgtcc

SEQ ID NO: 17 - protein, 658 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTRSPSKREPVVAVAAPASSESSSTNHSIGSTQSTPCSTSSMA

SEQ ID NO: 18 - protein, 625 aa, Homo sapiens
MDRGTLPLAVALLLASCSLSPTSLAETVHCDLQPVGPERGEVTYTTSQVSKGCVAQAPNAILEVHVLFLEFPTGPSQLELTLQASKQNGTWPREVLLVLSVNSSVFLHLQALGIPLHLAYNSSLVTFQEPPGVNTTELPSFPKTQILEWAAERGPITSAAELNDPQSILLRLGQAQGSLSFCMLEASQDMGRTLEWRPRTPALVRGCHLEGVAGHKEAHILRVLPGHSAGPRTVTVKVELSCAPGDLDAVLILQGPPYVSWLIDANHNMQIWTTGEYSFKIFPEKNIRGFKLPDTPQGLLGEARMLNASIVASFVELPLASIVSLHASSCGGRLQTSPAPIQTTPPKDTCSPELLMSLIQTKCADDAMTLVLKKELVAHLKCTITGLTFWDPSCEAEDRGDKFVLRSAYSSCGMQVSASMISNEAVVNILSSSSPQRKKVHCLNMDSLSFQLGLYLSPHFLQASNTIEPGQQSFVQVRVSPSVSEFLLQLDSCHLDLGPEGGTVELIQGRAAKGNCVSLLSPSPEGDPRFSFLLHFYTVPIPKTGTLSCTVALRPKTGSQDQEVHRTVFMRLNIISPDLSGCTSKGLVLPAVLGITFGAFLIGALLTAALWYIYSHTREYPRPPQ

SEQ ID NO: 19 - protein, 574 aa, Homo sapiens
MCPRAARAPATLLLALGAVLWPAAGAWELTILHTNDVHSRLEQTSEDSSKCVNASRCMGGVARLFTKVQQIRRAEPNVLLLDAGDQYQGTIWFTVYKGAEVAHFMNALRYDAMALGNHEFDNGVEGLIEPLLKEAKFPILSANIKAKGPLASQISGLYLPYKVLPVGDEVVGIVGYTSKETPFLSNPGTNLVFEDEITALQPEVDKLKTLNVNKIIALGHSGFEMDKLIAQKVRGVDVVVGGHSNTFLYTGNPPSKEVPAGKYPFIVTSDDGRKVPVVQAYAFGKYLGYLKIEFDERGNVISSHGNPILLNSSIPEDPSIKADINKWRIKLDNYSTQELGKTIVYLDGSSQSCRFRECNMGNLICDAMINNNLRHTDEMFWNHVSMCILNGGGIRSPIDERNNGTITWENLAAVLPFGGTFDLVQLKGSTLKKAFEHSVHRYGQSTGEFLQVGGIHVVYDLSRKPGDRVVKLDVLCTKCRVPSYDPLKMDEVYKVILPNFLANGGDGFQMIKDELLRHDSGDQDINVVSTYISKMKVIYPAVEGRIKFSTGSHCHGSFSLIFLSLWAVIFVLYQ